(19) United States
(12) Patent Application Publication
(10) Pub. No.: US 2006/0052301 A1
(43) Pub. Date: Mar. 9, 2006

(54) Splice variants of peptide YY, neuropeptide Y, pancreatic peptide Y and Amylin, and uses thereof

(76) Inventors: Ronen Shemesh, Modi'in (IL); Yossef Kliger, Rishon Le'Zion (IL); Lewis F. Neville, Rehovot (IL); Jeanne Bernstein, Kfar Yona (IL); Dani Eshel, Karkur (IL)

Correspondence Address: WINSTON & STRAWN LLP, 1700 K STREET, N.W., WASHINGTON, DC, 20006, US

(21) Appl. No.: 11/145,463

(22) Filed: Jun. 2, 2005

Related U.S. Application Data
(60) Provisional application No. 60/576,414, filed on Jun. 3, 2004.
(60) Provisional application No. 60/672,987, filed on Apr. 20, 2005.

(51) Int. Cl. A61K 38/22; C07K 14/575

(52) U.S. Cl. 514/12; 530/324

(57) ABSTRACT

The present invention relates to alternative splice variants of Amylin and members of the Pancreatic Polypeptide family, namely PYY, NPY, and PPY, vectors and compositions that include the same, and methods of use thereof. This invention provides peptides, nucleic acid sequences which encode same, analogs and derivatives thereof, antibodies which specifically recognize the variant sequences, compositions that include the same and methods of use thereof.

This application claims the benefit of US provisional patent applications 60/576,414 filed Jun. 3, 2004 and 60/672,987 filed Apr. 20, 2005, the entire contents of each of which is expressly incorporated herein by reference thereto.

### FIELD OF THE INVENTION

The present invention relates to alternative splice variants of peptides involved in regulation of energy metabolism, specifically Peptide YY (PYY), Neuropeptide Y (NPY), Pancreatic Peptide Y (PPY) and Amylin. This invention provides peptides, nucleic acid sequences which encode same, analogs and derivatives thereof, antibodies which specifically recognize the variant sequences, compositions comprising same and methods of use thereof, alone or in combination with additional regulatory peptides including but not limited to preproglucagon, glucagon-like peptide-1 (GLP-1) and oxyntomodulin (OXM).

### BACKGROUND OF THE INVENTION

Obesity and its associated disorders are common and very serious public health problems in the United States and throughout the world. Upper body obesity is the strongest risk factor known for diabetes mellitus type 2, and is a strong risk factor for cardiovascular disease. Obesity is a recognized risk factor for hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia (see, e.g., Kopelman, Nature 404: 635-43, 2000). It reduces life span and carries a serious risk of co-morbidities as described above, as well as disorders such as infections, varicose veins, acanthosis nigricans, eczema, exercise intolerance, insulin resistance, hypertension hypercholesterolemia, cholelithiasis, orthopedic injury, and thromboembolic disease (Rissanen et al., BMJ 301: 835-7, 1990). Obesity is also a risk factor for the group of conditions called insulin resistance syndrome, or “Syndrome X”.

Obesity is a chronic, essentially intractable metabolic disorder of ever-increasing prevalence for which no effective treatment is currently known. Therefore, clearly therapeutic treatments for obesity are very important.

Efforts to find such treatments have focused on a number of different areas, including but not limited to the use of naturally occurring hormones that have been shown to have effects in weight reduction. However, as described below, naturally occurring hormones have a number of deficiencies, such as lack of stability in the bloodstream.

A number of hormones have been identified as having regulatory effects in caloric intake, including leptin, preproglucagon, glucagon-like peptide, oxyntomodulin and the Pancreatic Polypeptide family.

A number of related hormones make up the Pancreatic Polypeptide (PP) family, including Peptide YY (PYY) (SwissProt accession: PYY_HUMAN; SEQ ID NO:65), Neuropeptide Y (NPY) (SwissProt accession: NEUY_HUMAN, SEQ ID NO:86), Pancreatic Peptide Y (PPY) (SwissProt accession: PAHO_HUMAN; SEQ ID NO:90) and Peptide YY-2 (PYY2) (RefSeq accession NM_{—}021093; SEQ ID NO:68). PYY is believed to be important in regulating glucose metabolism, feeding behavior and other metabolic functions. PYY is believed to also be useful for treating diabetes, and also possibly to stimulate the regrowth of pancreatic insulin-producing cells. Release of PYY occurs following a meal. An alternate molecular form of PYY is PYY[3-36] (SEQ ID NO:67) (Eberlein, et al., Peptides 10: 797-803, 1989; Grandt, et al., Regul Pept 51: 151-9, 1994), which is apparently a protease cleavage product of PYY. Signal peptidase releases the pre-pro polypeptide; peptide-convertase digests between RQR-GRK to form PYY[1-36] and DPP4 cleaves the two N-terminal residues to form PYY[3-36]. As described in greater detail below, the second cleavage of PYY results in an active product (PYY[1-36] (SEQ ID NO:66)), further cleavage at a third cleavage site also results in an active PYY product (PYY[3-36] (SEQ ID NO:67)), however with modified affinity to PYY receptors. Hereinafter, unless otherwise indicated, the term “PYY” refers to both active forms of known PYY, i.e. PYY[1-36] and PYY[3-36].

NPY and PYY are reported to have both opposing effects and varying degrees of selectivity for the “Y” receptors, which now number Y1-Y7 (Michel, et al., Pharmacol Rev 50:143-50, 1998; Gehlert, Proc Soc Exp Biol Med 218: 7-22, 1998). NPY prefers Y1, but also has some affinity for Y2 and Y5. PYY prefers Y1, Y2 and Y5. The affinity of PYY[3-36] is much higher for Y2 than for Y1 or Y5.

Like leptin, PYY interacts with two types of neurons, one that stimulates appetite (inhibitory effect) and one that inhibits appetite (stimulatory effect) in the arcuate nucleus. PYY switches off the release of NPY, which normally stimulates appetite. However, PYY, through interneuron connections, stimulates the release of the inhibitory a-MSH. Obese individuals have high levels of leptin, but are resistant to its effects. However, levels of PYY are low in obese individuals, and they remain sensitive to it. Peripheral administration of PYY has been shown to reduce gastric acid secretion, gastric motility, exocrine pancreatic secretion (Yoshinaga, et al., Am J Physiol 263: G695-701, 1992; Guan, et al., Endocrinology 128: 911-6, 1991; Pappas, et al., Gastroenterology 91: 1386-9, 1986), gallbladder contraction and intestinal motility (Savage, et al., Gut 28: 166-70, 1987). On the other hand, PYY has been shown to stimulate food and water intake after central administration (Morley, et al., Brain Res 341: 200-203, 1985; Corp, et al., Am J Physiol 259: R317-23, 1990).

PYY was recently shown to be effective in reduction of calorie intake in an initial clinical trial (Batterham et al., N. Eng. J. Med., 349: 941-948, 2003). Various effects were seen, including reduction of ghrelin levels (an appetite stimulant), reduction of endogeneous fasting and post-meal PYY levels, and negative correlation of PYY with body mass index. Therefore, obese subjects do not show any resistance to the anorectic effects of PYY. No side effects were observed. The potential uses of PYY and PYY agonists have been also described in US2002/0141985 to Amylin Pharmaceuticals Inc., hereby incorporated by reference as if fully set forth herein. This application teaches the use of PYY itself and a known variant, PYY[3-36], which lacks the first 2 amino acids of the known wild type (WT) sequence. It is one measure of the importance of locating PYY agonists that various applications refer to such an agonist, yet the only agonist taught is the previously described known variant of PYY, which is PYY[3-36]. Despite the clear importance of PYY agonists, no other effective agonists are currently taught in the art.

Unfortunately, PYY and PYY[3-36] are both highly unstable in the blood (PYY[3-36] for example, has a half-life of around 3 hours), as they are both quickly deactivated by a protease, which without wishing to be limited by a single hypothesis, may be dipeptidyl peptidase. Therefore, clearly new PYY agonists are required which have a higher degree of stability in the blood, while still having the beneficial effects for treatment of obesity and/or other metabolic disorders.

Based on a comparison of the gene sequences, it is believed that NPY and PYY are the results of a gene duplication event, and that subsequent tandem duplication produced the PPY gene. For all three gene family members an alternate molecular form exists, derived from a protease cleavage: PYY[3-36] (SEQ ID NO:67); NPY[3-36] (SEQ ID NO:102) and PPY[3-36] (SEQ ID NO:106). Similarly to PYY described above, the second cleavage of pre-pro peptides of NPY and PPY may result in an active product NPY[1-36] (SEQ ID NO:100) and PPY[1-36] (SEQ ID NO:104), cleavage at a third cleavage site results in NPY[3-36] (SEQ ID NO:102) and PPY[3-36] (SEQ ID NO:106). Hereinafter, unless otherwise indicated, the terms “NPY” and “PPY” refer to active forms of known NPY and PPY, respectively, i.e. NPY[1-36], NPY[3-36], PPY[1-36] and PPY[3-36].

Neuropeptide Y (NPY) is very highly conserved in a variety of animal, reptile and fish species. It is found in many central and peripheral sympathetic neurons and is the most abundant peptide observed in the mammalian brain. The peptide has been found to elicit a number of physiological responses including appetite stimulation, anxiolysis, hypertension, and the regulation of coronary tone. Reducing the NPY synthesis, secretion and function, e.g. using NPY antagonists, was shown to result in a reduced appetite and a regulated metabolism that enhance weight control, including weight loss and reduction of obesity as disclosed in U.S. Pat. No. 6,348,472, U.S. Pat. No. 6,177,429, and U.S. Pat. No. 6,013,622.

In addition to its role in metabolism regulation and eating response, NPY acts as potent vasoconstrictor, potentiating and prolonging the effects of norepinephrine and other vasoconstrictors, and blocker of vasodilators (Thorsell et al., Neuropeptides 36:182-93, 2002); angiogenesis simulator (Lee et all., J Clin Invest. 111:1853-62, 2003); and modulator of inflammation. NPY facilitates binding of leukocytes to both endothelial tissue and fibronectin (Levite et al., J Immunol. 160:993-1000, 1998), release of histamine from mast cells (Shen et al, Eur J Pharmacol. 204:249-56, 1991) increase secretion of TNF-α and IL-1,β, superoxide and IL-6 (Hernanz et al., J Neuroimmunol. 71:25-30, 1996). Administration of the NPY antagonist BIBP3226 has been shown to reduce swelling and inflammation associated with capsaicin in the mouse ear (Naveilhan et al., Nature, 409:513-7, 2001), which is a classic inflammation model, thereby indicating that blocking NPY activity may reduce inflammation.

Pancreatic peptide Y (PPY) is the third member of the PP family. Its physiological significance is not completely known, however, similarly to PYY agonists, PPY is believed to possess activity as an agent that reduces nutrient availability, including reduction of food intake, as disclosed for example in US20050009748.

Amylin (Islet or insulinoma amyloid polypeptide; IAPP_HUMAN) is a 37-amino acid monomeric polypeptide isolated from pancreatic amyloid (Cooper et al., Proc Natl Acad Sci USA., 84:8628-32, 1987). Such amyloids are commonly found in the islets of patients with diabetes mellitus type 2 and in insulinomas. Specific immunoreactivity with antibodies to IAPP is found in islet amyloid and in cells of the islets of Langerhans, where it colocalizes with insulin in islet B cells. Amylin is a peptide hormone that is co-secreted with insulin from the pancreatic beta cells. Human IAPP gene contains 3 exons (Hoppener et al., J Cell Biochem., 55 Suppl:39-53, 1994), encoding precursor proteins of 89 amino acids. Sequences for the known Amylin protein/polynucleotide are shown in SEQ ID NO:93 and 92 (protein and mRNA, respectively). Amylin and insulin work together with glucagons to maintain normal glucose concentrations. Insulin and Amylin concentrations normally increase while glucagon levels decrease after meals. The anorectic action of Amylin is summarized in a recent review by Lutz et al (Curr Drug Targets, 6:181-189, 2005). Amylin is released during meals, and exogenous Amylin leads to a dose-related reduction in meal size. Amylin has a rapid onset and brief duration of action. It inhibits glucagon secretion, delays gastric emptying, and acts as a satiety agent.

In type 1 diabetes, Amylin has been shown to be missing or deficient and combined replacement with insulin has been proposed as a preferred treatment over insulin alone in all forms of diabetes. It has been proposed that the lack of Amylin contributes to poor glucose control, especially after eating. Indeed, Amylin has been shown to have at least two effects believed to be important for normal glucose metabolism: it slows glucose inflow into the bloodstream from the gastrointestinal tract, and it suppresses glucagon secretion and thereby helps to lower glucose production by the liver. The use of Amylin and other Amylin agonists for the treatment of diabetes mellitus is disclosed in U.S. Pat. No. 5,175,145. Pharmaceutical compositions containing Amylin and Amylin plus insulin are described in U.S. Pat. No. 5,124,314.

Amylin replacement could therefore possibly improve glycemic control in patients with diabetes. However, human Amylin exhibits physicochemical properties predisposing the peptide hormone to aggregate and form amyloid fibers, which may play a part in beta-cell destruction in type 2 diabetes. This obviously makes it unsuitable for pharmacological use. A stable analog, pramlintide acetate, (known as SYMLIN™) which has actions and pharmacokinetic and pharmacodynamic properties similar to the native peptide, has been developed by Amylin Pharmaceuticals, Inc. (San Diego, California) and recently approved by the FDA for treatment of type 1 and type 2 diabetes. Clinical studies demonstrated that SYMLIN™, used as a self-administered injection given prior to meals, helps patients to achieve lower blood glucose levels after the meal and less fluctuation during the day, and better long term glucose control compared to the patients taking insulin alone. However, “SYMLIN™ has been associated with an increased risk of insulin-induced severe hypoglycemia, particularly in patients with type 1 diabetes (http://www.amylin.com/pipeline/symlin.cfm).

Exogenous Amylin potently and dose-dependently reduces feeding in rats and mice, with both central and peripheral sites being effective. Amylin has been characterized as a satiety signal that regulates short-term food intake (i.e., meal size), and recent data indicate that Amylin may have long term effects on food intake and body weight (Rushing, Curr Pharm Des., 9:819-25, 2003). Methods for using Amylin or Amylin agonists alone or in conjunction with another obesity relief agent for reducing food intake, suppressing appetite and controlling body weight are disclosed for example in WO98/55144 and EP0844882, hereby incorporated by reference as if fully set forth herein.

Potential uses of Amylin for treating anorexia and related states, such as cachexia and adipose tissue deficiency, are described in EP0586589, hereby incorporated by reference as if fully set forth herein. EP0586589 teaches that a patient suffering from anorexia may have fasting plasma Amylin and insulin concentrations below the normal range, and in fact near the range measured for type 1 diabetes. Patients suffering from cachexia or receiving parenteral nutrition have reduced Amylin and/or insulin levels. Thus, it has been proposed that patients suffering from anorexia and cachectic states, as well as patients undergoing parenteral nutrition, be administered Amylin with or without insulin. Such administration will preferably increase adipose tissue in such patients and thus be of significant benefit.

Amylin was shown to inhibit gastric acid secretion. It protects the gastric mucosa in ulcer models like stress, vagal stimulation, ethanol, acetic acid, reserpine and serotonine administration and pylorus ligation. This protective antiulcer is seen not only at pharmacological but also at near-physiological doses of 0.5 μg/kg. Moreover Amylin also was shown to exert curative properties in the acetic acid and indomethacin ulcer models. It decreases the aggressive factors like acid-pepsin secretion, increases mast cell stability and increases protective mechanisms like bicarbonate gastric secretion, dilates blood vessels, and it increases lymphatic mesenteric activity. Amylin seems to be a powerful protector of gastric mucosa in animals by increasing the stability of gastric mucosa (Samonina, et al., Pathophysiology 11:1-6, 2004). Using Amylin or Amylin agonists for treating or preventing gastritis or gastric ulceration is described in EP0981360, hereby incorporated by reference as if fully set forth herein.

Non-metabolic actions of Amylin include vasodilator effects, stimulation of the proliferation of osteoblasts in vitro, inducing of bone formation, treating pancreatitis and relieving pain. The vasodilator effects of Amylin may be mediated by interaction with CGRP 6 vascular receptors. The effect of Amylin on regional hemodynamic actions, including renal blood flow, in conscious rats has been reported (Gardiner et al., Diabetes, 40:948-951, 1991). The authors noted that infusion of rat Amylin was associated with greater renal vasodilation and less mesenteric vasoconstriction than is seen with infusion of human a-CGRP. Use of Amylin as a vasodilator is described in EP0289287. Amylin has also been reported to have effects both on isolated osteoclasts where it caused cell quiescence, and in vivo where it was reported to lower plasma calcium by up to 20% in rats, in rabbits, and in humans with Paget's disease (Zaidi et, al., Trends in Endocrinal. and Metab., 4:255-259, 1993). It can stimulate the proliferation of osteoblasts in vitro, and causes measurable bone formation when administered either locally or systemically in vivo. In addition, Amylin inhibits bone resorption. Amylin has also been found to act on chondrocytes, stimulating their proliferation in culture and increasing tibial growth plate thickness when administered systemically to adult mice (Cornish et al., Curr Pharm Des.,8:2009-21, 2002). Amylin is also reported as having value for treatment of bone disorders and calcium imbalance. The activity of Amylin is attributed in EP408284 to an inhibition of osteoclast motility. Administration of Amylin was found to stimulate bone growth. Using Amylin or Amylin agonists as an agent for treatment of bone disorders where stimulation of bone growth is required, such as osteoporosis, Paget's disease, malignant deposits in bone, bone loss of malignancy, or endocrine disorders, arthritis, immobility, treating fractures, and other conditions where a hypocalcemic effect is of benefit, is disclosed. in U.S. Pat. No. 5,922,677; U.S. Pat. No. 6,821,954 and EP0408284

Potential uses of Amylin or Amylin agonists for treating pancreatitis or relieving pain caused by pancreatitis is described in WO04/037168, hereby incorporated by reference as if fully set forth herein.

Potential uses of Amylin or Amylin agonists for treatment or prevention of pain, e.g. migraine, optionally with a narcotic analgesic or other pain relief agents is described in U.S. Pat. No. 5,677,279, hereby incorporated by reference as if fully set forth herein.

Because of their roles in regulating metabolism, PYY, PPY, NPY and Amylin variants, with comparable function, yet fewer limitations to their use, are necessary, and currently lacking.

### SUMMARY OF INVENTION

The present invention provides PYY variants, PPY variants, NPY variants and Amylin variants, compositions comprising same and methods of use thereof. Methods of use include but are not limited to, affecting a metabolic state in a subject, including treating a metabolic regulatory imbalance and/or restoring metabolic balance in a subject, regulating the metabolism of a subject, treating of a disease or a condition having a pathology related to a metabolic activity, wherein a beneficial therapeutic effect is achieved by metabolic regulation, treating metabolic conditions or disorders, particularly those which can be alleviated by reducing caloric availability and/or any mechanism for which PYY and/or NPY and/or PPY and/or Amylin treatment may be suitable.

According to certain embodiments, the metabolic state, disease or condition is selected from obesity, diabetes mellitus, eating disorders, regulation of appetite, feeding behaviour, stress related feeding disorders, conditions or disorders which can be alleviated by improving lipid profile, dislipidemia, conditions or disorders which can be alleviated by reducing nutrient intake or availability, reducing degeneration of pancreatic tissue, altering the differentiated state of a pancreatic islet or cell, regulation of islet cell secretion, disease associated with altered glucose metabolism and/or glucose homeostasis, glucose intolerance, insulin-resistance syndrome (Syndrome X), gastric emptying, intestinal growth, intestinal evacuation, hypertension, and cardiovascular diseases.

The term “metabolic state” refers to the overall metabolism of the body of a subject, including whole body metabolism, as well as to the function of particular organs which impact upon the metabolism of a subject, optionally and preferably including metabolism of carbohydrates and fats in the body of the subject. “Affecting” metabolic state includes but is not limited to, upregulating or downregulating metabolism, or controlling metabolism.

The term “condition or disorder which can be alleviated by reducing caloric intake or availability” is meant to encompass any condition or disorder in a subject that is caused by, complicated by, or aggravated by a relatively high nutrient availability, or that can be alleviated by reducing nutrient availability, for example by decreasing food intake. Such conditions or disorders include, but are not limited to, obesity, diabetes, including type 2 diabetes, eating disorders, and insulin-resistance syndromes.

The term “metabolic regulatory imbalance” is meant to encompass any condition or disorder in a subject that is caused by, complicated by, or aggravated by an unbalanced energy levels or any other failure to achieve metabolic regulation or balance. Such conditions or disorders include, but are not limited to, glucose homeostasis, gastric emptying, intestinal growth, regulation of islet cell secretion, regulation of appetite, obesity, diabetes, including type 2 diabetes, eating disorders, and insulin-resistance syndromes, hypertension and cardiovascular diseases.

The term “restoring metabolic balance” is meant to achieve the state of metabolic balance in a subject who is in a state of metabolic imbalance. Restoring metabolic balance is preferably used to treat conditions or disorders that include, but are not limited to, glucose homeostasis, gastric emptying, intestinal growth, regulation of islet cell secretion, regulation of appetite, obesity, diabetes, including type 2 diabetes, eating disorders, and insulin-resistance syndromes, hypertension and cardiovascular diseases.

According to certain embodiments of the present invention, the invention provides a method of treating obesity in an obese or overweight subject by administering a therapeutically effective amount of a at least one of the variants of the present invention, alone or in combination according to the present invention. While “obesity” is generally defined as a body mass index over 30, for purposes of the present invention, any subject, including those with a body mass index of less than 30, who needs or wishes to reduce body weight is included in the scope of “obese”. Subjects who are insulin resistant, glucose intolerant, or have any form of diabetes mellitus (e.g., type 1, 2 or gestational diabetes) and/or any other metabolic disorder resulting in excessive weight gain and/or maintenance of excessive weight can benefit from the compositions of the present invention.

In one aspect, the present invention provides an isolated PYY splice variant polypeptide having an amino acid sequence as set forth in any one of SEQ ID NOS:70-75, or a fragment thereof.

According to certain embodiments, the invention provides an isolated PYY splice variant polypeptide, wherein the polypeptide is encoded by a nucleic acid sequence as set forth in any one of SEQ ID NOS:80-83, or a fragment thereof.

According to other embodiments, the present invention relates to analogs, homologs and derivatives of the PYY splice variants as set forth in any one of SEQ ID NOS:70-75.

According to other embodiments, the invention provides an antibody specifically recognizing the isolated PYY splice variants and polypeptide. fragments of this invention. Preferably such an antibody differentially recognizes PYY splice variants of the present invention but do not recognize known PYY peptides.

According to other embodiments, the invention provides an isolated nucleic acid encoding for a PYY splice variant, having a nucleotide sequence as set forth in any one of SEQ ID NOS:80-83, or a sequence complementary thereto.

In another embodiment, the invention provides an oligonucleotide of at least about 12 nucleotides, wherein said oligonucleotide is specifically hybridizable with the nucleic acid molecules, encoding PYY variants of this invention.

According to other embodiments, the invention provides compositions, cells, liposomes, and/or vectors comprising the nucleic acids of this invention.

According to other embodiments, the invention provides a pharmaceutical composition comprising as an active ingredient a PYY splice variant polypeptide having an amino acid sequence as set forth in any one of SEQ ID NOS:70-75, or a fragment thereof.

According to other embodiments, the invention provides a pharmaceutical composition comprising as an active ingredient a PYY splice variant having a nucleotide sequence as set forth in any one of SEQ ID NOS:80-83.

In another embodiment, the invention provides a method for detecting PYY splice variant nucleic acid sequences in a biological sample, comprising the steps of: hybridizing isolated nucleic acid molecules of this invention, or oligonucleotide fragments of at least about 12 nucleotides thereof to a nucleic acid material of the biological sample and detecting the hybridization complex; wherein the presence of a hybridization complex correlates with the presence of a splice variant nucleic acid sequence in the biological sample.

In another embodiment, the invention provides a method for detecting PYY splice variants in a biological sample, comprising the steps of contacting the biological sample with an antibody specifically recognizing the isolated PYY splice variant polypeptide under conditions whereby the antibody specifically interacts with a PYY splice variant polypeptide in the biological sample but do not recognize known PYY peptides, and detecting the interaction; wherein the presence of the interaction correlates with the presence of a splice variant in the biological sample.

The PYY splice variants, as described above, can be used for affecting a metabolic state in a subject, including treating a metabolic regulatory imbalance and/or restoring metabolic balance in a subject and/or regulating metabolism of a subject and/or treating of a disease or a condition having a pathology related to a metabolic activity wherein a beneficial therapeutic effect is achieved by metabolic regulation and/or any mechanism for which PYY treatment is suitable. According to certain embodiments, the metabolic state or disease or condition is selected from obesity, diabetes mellitus, eating disorders, regulation of appetite, feeding behaviour, stress related feeding disorders, conditions or disorders which can be alleviated by improving lipid profile, dislipidemia, conditions or disorders which can be alleviated by reducing nutrient intake or availability, reducing degeneration of pancreatic tissue, altering the differentiated state of a pancreatic islet or cell, regulation of islet cell secretion, disease associated with altered glucose metabolism and/or glucose homeostasis, insulin resistance, gastric emptying, intestinal evacuation, intestinal growth, hypertension, cardiovascular diseases. The PYY treatment comprising administering to said subject a therapeutically effective amount of a PYY variant. The PYY treatment can be administered alone or in combination with at least one of the other splice variants of the present invention as described herein, and/or in combination with at least one of GLP-1, OXM and preproglucagon variants, and/or in combination with any other agent known to be involved in glucose control and/or metabolic regulation. Preferably, the PYY variants are used (alone or in combination with a GLP-1 splice variant and/or known GLP-1 and/or OXM splice variant and/or known OXM and/or with PPY splice variants and/or known PPY, and/or NPY variants and/or Amylin variants and/or known Amylin and/or known Amylin agonists) for treatment of any condition or disorder which can be alleviated by reducing caloric intake or availability, as described above.

According to certain embodiments of the present invention, the invention provides a method of treating obesity in an obese or overweight subject by administering a therapeutically effective amount of a PYY variant, alone or in combination according to the present invention. According to certain preferred embodiments of the present invention, the PYY variants and methods of the invention are used to treat conditions or disorders which can be alleviated by reducing nutrient availability in a subject in need thereof, comprising administering to said subject a therapeutically effective amount of a PYY variant. Such conditions and disorders include, but are not limited to, hypertension, dyslipidemia, hyperglycemia, cardiovascular disease, eating disorders, insulin-resistance, obesity, and diabetes mellitus of any kind.

According to further preferred embodiments, this invention provides a method of using PYY splice variants for post surgery treatments, including but not limited to attenuating post-surgical catabolic changes, hormonal responses to stress and insulin resistance.

According to other embodiments, this invention provides a method for treating a metabolic regulatory imbalance in a subject, comprising administering to a patient in need thereof a PYY splice variant having an amino acid sequence as set forth in any one of SEQ ID NOS:70-75 or a derivative thereof.

According to other embodiments, this invention provides a method for treating a metabolic regulatory imbalance in a subject comprising administering to the subject an isolated nucleic acid encoding a PYY splice variant, wherein the PYY splice variant has an amino acid sequence as set forth in any one of SEQ ID NOS:70-75 or a derivative thereof.

According to other embodiments, this invention provides a method for treating diabetes in a subject comprising administering to the subject a PYY splice variant having an amino acid sequence as set forth in any one of SEQ ID NOS:70-75, or a derivative thereof, wherein the PYY splice variant is insulinotropic in the subject, thereby treating maturity onset diabetes mellitus in said subject.

According to other embodiments, this invention provides a method for treating diabetes in a subject comprising administering to the subject an isolated nucleic acid encoding a PYY splice variant, wherein the PYY splice variant has an amino acid sequence as set forth in any one of SEQ ID NOS:70-75 or a derivative thereof, and wherein the PYY splice variant is insulinotropic in the subject, thereby treating maturity onset diabetes mellitus in said subject.

According to another aspect, the present invention provides an isolated NPY splice variant polypeptide having an amino acid sequence as set forth in any one of SEQ ID NOS:87, 101 or 103.

In one embodiment, the invention provides an isolated NPY splice variant polypeptide, wherein the polypeptide is encoded by a nucleic acid sequence as set forth in SEQ ID NO:85.

According to other embodiments, the present invention relates to NPY splice variant analogs, homologs and derivatives of the NPY splice variants as set forth in any one of SEQ ID NOS:87, 101 or 103.

According to other embodiments, the invention provides an antibody specifically recognizing the isolated NPY splice variants and polypeptide fragments of this invention. Preferably such an antibody differentially recognizes NPY splice variants of the present invention but do not recognize known NPY peptides.

According to other embodiments the invention provides an isolated nucleic acid encoding for a NPY splice variant, having a nucleotide sequence as set forth in SEQ ID NO:85, or a sequence complementary thereto.

In another embodiment, the invention provides an oligonucleotide of at least about 12 nucleotides, wherein said oligonucleotide is specifically hybridizable with the nucleic acid molecules encoding NPY variant of this invention.

According to other embodiments, the invention provides compositions, cells, liposomes, and/or vectors comprising the nucleic acids of this invention.

According to other embodiments, the invention provides a pharmaceutical composition comprising as an active ingredient an NPY splice variant polypeptide having an amino acid sequence as set forth in any one of SEQ ID NOS:87, 101, 103, or a fragment thereof.

In another embodiment, the invention provides a pharmaceutical composition comprising as an active ingredient a NPY splice variant having a nucleotide sequence as set forth in SEQ ID NO:85.

In another embodiment, the invention provides a method for detecting NPY splice variant nucleic acid sequences in a biological sample, comprising the steps of: hybridizing isolated nucleic acid molecules of this invention, or oligonucleotide fragments of at least about 12 nucleotides thereof to a nucleic acid material of the biological sample and detecting the hybridization complex; wherein the presence of a hybridization complex correlates with the presence of a splice variant nucleic acid sequence in the biological sample.

In another embodiment, the invention provides a method for detecting NPY splice variants in a biological sample, comprising the steps of contacting the biological sample with an antibody specifically recognizing the isolated NPY splice variant polypeptide under conditions whereby the antibody specifically interacts with a NPY splice variant polypeptide in the biological sample but do not recognize known NPY peptides, and detecting the interaction; wherein the presence of the interaction correlates with the presence of a splice variant in the biological sample.

The NPY splice variants, as described above, are capable of treating a metabolic regulatory imbalance, preferably having an antagonistic activity as compared to the known NPY. The NPY splice variants, as described above, can be used for affecting a metabolic state in a subject, including treating a metabolic regulatory imbalance and/or restoring metabolic balance in a subject and/or regulating metabolism of a subject and/or treating of a disease or a condition having a pathology related to a metabolic activity wherein a beneficial therapeutic effect is achieved by metabolic regulation and/or any mechanism for which NPY treatment is suitable.

According to certain embodiments, the metabolic state or disease or condition is selected from obesity, diabetes mellitus, eating disorders, feeding behaviour, stress related feeding disorders, regulation of appetite, conditions or disorders which can be alleviated by improving lipid profile, dislipidemia, hyperglycemia, conditions or disorders which can be alleviated by reducing nutrient intake or availability, reducing degeneration of pancreatic tissue, altering the differentiated state of a pancreatic islet or cell, regulation of islet cell secretion, disease associated with altered glucose metabolism and/or glucose homeostasis, insulin resistance, gastric emptying, intestinal evacuation, intestinal growth, hypertension, cardiovascular diseases.

The NPY treatment comprises administering to said subject a therapeutically effective amount of an NPY variant, having an antagonistic mode of action comparing to the known NPY. The NPY treatment can be administered alone or in combination with at least one of the other splice variants of the present invention as described herein, and/or in combination with at least one of GLP-1, OXM and preproglucagon variants, and/or in combination with any other agent known to be involved in glucose control and/or metabolic regulation. Preferably, the NPY variants are used (alone or in combination with a GLP-1 splice variant and/or known GLP-1 and/or OXM splice variant and/or known OXM and/or with PYY splice variants and/or known PYY, and/or with PPY splice variants and/or known PPY, and/or Amylin variants and/or known Amylin and/or known Amylin agonists) for treatment of any condition or disorder which cain be alleviated by reducing caloric intake or availability, as described above.

According to other embodiments, this invention provides a method of using NPY splice variants for post surgery treatments, including but not limited to attenuating post-surgical catabolic changes, hormonal responses to stress and insulin resistance.

According to other embodiments, this invention provides a method of using NPY splice variants for preventing and/or treating inflammatory conditions in a subject, such as cutaneous, internal and/or neurogenic inflammation, including treatment of acute or chronic/persistent inflammation.

According to other embodiments, this invention provides a method of using NPY splice variants for treating or preventing disorders related to angiogenesis, excessive formation of vascular tissue or blood vessels, including neovascular glaucoma, retinopathy, nephropathy, a cardiovascular disease or a cancerous disease.

According to other embodiments, this invention provides a method of using NPY splice variants for preventing and/or treating of male erectile dysfunction.

According to other embodiments, this invention provides a method for treating a metabolic regulatory imbalance in a subject, comprising administering to a patient in need thereof a NPY splice variant having an amino acid sequence as set forth in any one of SEQ ID NOS: 87, 101, 103 or a derivative thereof.

According to other embodiments, this invention provides a method for treating a metabolic regulatory imbalance in a subject comprising administering to the subject an isolated nucleic acid encoding a NPY splice variant, wherein the NPY splice variant has an amino acid sequence as set forth in any one of SEQ ID NOS:87, 101, 103 or a derivative thereof.

According to other embodiments, the invention provides a method for treating diabetes in a subject comprising administering to the subject a NPY splice variant having an amino acid sequence as set forth in any one of SEQ ID NOS:87, 101, 103 or a derivative thereof, wherein the NPY splice variant is insulinotropic in the subject, thereby treating maturity onset diabetes mellitus in the subject.

According to other embodiments, the invention provides a method for treating diabetes in a subject comprising administering to the subject an isolated nucleic acid encoding a NPY splice variant, wherein the NPY splice variant has an amino acid sequence as set forth in any one of SEQ ID NOS:87, 101, 103 or a derivative thereof, and wherein the NPY splice variant is insulinotropic in the subject, thereby treating maturity onset diabetes mellitus in the subject.

According to another aspect, the present invention provides an isolated PPY splice variant polypeptide having an amino acid sequence as set forth in any one of SEQ ID NOS:91, 105 or 107.

In another embodiment, the invention provides an isolated PPY splice variant polypeptide, wherein the polypeptide is encoded by a nucleic acid sequence as set forth in SEQ ID NO:89.

According to other embodiments, the present invention relates to analogs, homologs and derivatives of the PPY splice variants as set forth in any one of SEQ ID NOS:91, 105 or 107.

According to other embodiments, the invention provides an antibody specifically recognizing the isolated PPY splice variants and polypeptide fragments of this invention. Preferably such an antibody differentially recognizes PPY splice variants of the present invention but do not recognize known PPY peptides.

According to other embodiments the invention provides an isolated nucleic acid encoding for a PPY splice variant, having a nucleotide sequence as set forth in SEQ ID NO:89, or a sequence complementary thereto.

According to other embodiments, the invention provides an oligonucleotide of at least about 12 nucleotides, wherein said oligonucleotide is specifically hybridizable with the nucleic acid molecules of this invention. According to other embodiments, this invention provides compositions, cells, liposomes, and/or vectors comprising the nucleic acids of this invention.

According to other embodiments, the invention provides a pharmaceutical composition comprising as an active ingredient a PPY splice variant polypeptide having an amino acid sequence as set forth in any one of SEQ ID NOS:91, 105, 107, or a fragment thereof.

According to other embodiments, the invention provides a pharmaceutical composition comprising as an active ingredient a PPY splice variant having a nucleotide sequence as set forth in any one of SEQ ID NOS:89.

In another embodiment, the invention provides a method for detecting PPY splice variant nucleic acid sequences in a biological sample, comprising the steps of: hybridizing isolated nucleic acid molecules of this invention, or oligonucleotide fragments of at least about 12 nucleotides thereof to a nucleic acid material of the biological sample and detecting the hybridization complex; wherein the presence of a hybridization complex correlates with the presence of a splice variant nucleic acid sequence in the biological sample.

In another embodiment, the invention provides a method for detecting PPY splice variants in a biological sample, comprising the steps of contacting the biological sample with an antibody specifically recognizing the isolated PPY splice variant polypeptide under conditions whereby the antibody specifically interacts with a PPY splice variant polypeptide in the biological sample but do not recognize known PPY peptides, and detecting the interaction; wherein the presence of the interaction correlates with the presence of a splice variant in the biological sample.

The PPY splice variants, as described above, can be used for affecting a metabolic state in a subject, including treating a metabolic regulatory imbalance and/or restoring metabolic balance in a subject and/or regulating metabolism of a subject and/or treating of a disease or a condition having a pathology related to a metabolic activity wherein a beneficial therapeutic effect is achieved by metabolic regulation and/or any mechanism for which PPY treatment is suitable.

According to certain embodiments, the metabolic state or disease or condition is selected from obesity, diabetes mellitus, eating disorders, feeding behaviour, stress related feeding disorders, regulation of appetite, conditions or disorders which can be alleviated by improving lipid profile, dislipidemia, hyperglycemia, conditions or disorders which can be alleviated by reducing nutrient intake or availability, reducing degeneration of pancreatic tissue, altering the differentiated state of a pancreatic islet or cell, regulation of islet cell secretion, disease associated with altered glucose metabolism and/or glucose homeostasis, insulin resistance, gastric emptying, intestinal evacuation, intestinal growth, hypertension, cardiovascular diseases.

The PPY treatment comprises administering to said subject a therapeutically effective amount of a PPY variant. The PPY treatment can be administered alone or in combination with at least one of the other splice variants of the present invention as described herein, and/or in combination with at least one of GLP-1, OXM and preproglucagon variants, and/or in combination with any other agent known to be involved in glucose control and/or metabolic regulation. Preferably, the PPY variants are used (alone or in combination with a GLP-1 splice variant and/or known GLP-1 and/or OXM splice variant and/or known OXM and/or with PYY splice variants and/or known PYY, and/or NPY variants and/or Amylin variants and/or known Amylin and/or known Amylin agonists) for treatment of any condition or disorder which can be alleviated by reducing caloric intake or availability, as described above.

According to other embodiments, this invention provides a method for treating a metabolic regulatory imbalance in a subject, comprising administering to a patient in need thereof a PPY splice variant having an amino acid sequence as set forth in any one of SEQ ID NOS:91, 105, 107 or a derivative thereof.

According to other embodiments, this invention provides a method for treating a metabolic regulatory imbalance in a subject comprising administering to the subject an isolated nucleic acid encoding a PPY splice variant, wherein the PPY splice variant has an amino acid sequence as set forth in any one of SEQ ID NOS: 91, 105, 107 or a derivative thereof.

In another aspect, the present invention provides an isolated Amylin splice variant polypeptide having an amino acid sequence as set forth in any one of SEQ ID NOS:95 or 97.

According to other embodiments, the invention provides an isolated Amylin splice variant polypeptide, wherein the polypeptide is encoded by a nucleic acid sequence as set forth in any one of SEQ ID NOS:94 or 96.

According to other embodiments, the present invention relates to analogs, homologs and derivatives of the Amylin splice variants as set forth in any one of SEQ ID NOS:95 or 97.

According to other embodiments, the invention provides an antibody specifically recognizing the isolated Amylin splice variants and polypeptide fragments of this invention. Preferably such an antibody differentially recognizes Amylin splice variants of the present invention but do not recognize known Amylin peptides.

According to other embodiments the invention provides an isolated nucleic acid encoding for a Amylin splice variant, having a nucleotide sequence as set forth in any one of SEQ ID NOS:94, 96, or a sequence complementary thereto.

In another embodiment, the invention provides an oligonucleotide of at least about 12 nucleotides, wherein said oligonucleotide is specifically hybridizable with the nucleic acid molecules, encoding Amylin variants of this invention.

According to other embodiments, the invention provides compositions, cells, liposomes, and/or vectors comprising the nucleic acids of this invention.

According to other embodiments, the invention provides a pharmaceutical composition comprising as an active ingredient a Amylin splice variant polypeptide having an amino acid sequence as set forth in any one of SEQ ID NOS:95, 97, or a fragment thereof.

According to other embodiments, the invention provides a pharmaceutical composition comprising as an active ingredient a Amylin splice variant having a nucleotide sequence as set forth in any one of SEQ ID NOS:94 or 96.

In another embodiment, the invention provides a method for detecting Amylin splice variant nucleic acid sequences in a biological sample, comprising the steps of: hybridizing isolated nucleic acid molecules of this invention, or oligonucleotide fragments of at least about 12 nucleotides thereof to a nucleic acid material of the biological sample and detecting the hybridization complex; wherein the presence of a hybridization complex correlates with the presence of a splice variant nucleic acid sequence in the biological sample.

In another embodiment, the invention provides a method for detecting Amylin splice variants in a biological sample, comprising the steps of contacting the biological sample with an antibody specifically recognizing the isolated Amylin splice variant polypeptide under conditions whereby the antibody specifically interacts with a Amylin splice variant polypeptide in the biological sample but do not recognize known Amylin peptides, and detecting the interaction; wherein the presence of the interaction correlates with the presence of a splice variant in the biological sample.

The Amylin splice variants, as described above, can be used for affecting a metabolic state in a subject, including treating a metabolic regulatory imbalance and/or restoring metabolic balance in a subject and/or regulating metabolism of a subject and/or treating of a disease or a condition having a pathology related to a metabolic activity wherein a beneficial therapeutic effect is achieved by metabolic regulation and/or any mechanism for which Amylin treatment is suitable.

According to certain embodiments, the metabolic state or disease or condition is selected from obesity, diabetes mellitus, eating disorders, feeding behaviour, stress related feeding disorders, regulation of appetite, conditions or disorders which can be alleviated by improving lipid profile, dislipidemia, conditions or disorders which can be alleviated by reducing nutrient intake or availability, reducing degeneration of pancreatic tissue, altering the differentiated state of a pancreatic islet or cell, regulation of islet cell secretion, disease associated with altered glucose metabolism and/or glucose homeostasis, insulin resistance, gastric emptying, intestinal evacuation, intestinal growth, hypertension, cardiovascular diseases.

The Amylin treatment comprises administering to said subject a therapeutically effective amount of an Amylin variant. The Amylin treatment can be administered alone or in combination with at least one of the other splice variants of the present invention as described herein, and/or in combination with at least one of GLP-1, OXM and preproglucagon variants, and/or in combination with any other agent known to be involved in glucose control and/or metabolic regulation. Preferably, the Amylin variants are used (alone or in combination with a GLP-1 splice variant and/or known GLP-1 and/or OXM splice variant and/or known OXM and/or with PPY splice variants and/or known PPY, and/or NPY variants and/or PPY variants and/or known PPY) for treatment of any condition or disorder which can be alleviated by reducing caloric intake or availability, as described above.

According to other embodiments, this invention provides a method for treating a metabolic regulatory imbalance in a subject, comprising administering to a patient in need thereof Amylin splice variant having an amino acid sequence as set forth in any one of SEQ ID NOS:95, 97 or a derivative thereof.

According to other embodiments, this invention provides a method for treating a metabolic regulatory imbalance in a subject comprising administering to the subject an isolated nucleic acid encoding Amylin splice variant, wherein the Amylin splice variant has an amino acid sequence as set forth in any one of SEQ ID NOS:95, 97 or a derivative thereof.

According to other preferred embodiments of the present invention, the Amylin variants and methods of the invention are used for treating or preventing gastritis or gastric ulceration in a subject, comprising administering to said subject a therapeutically effective amount of an Amylin variant.

According to other preferred embodiments of the present invention, the Amylin variants and methods of the invention are used to treat conditions or disorders which can be alleviated by Amylin as vasodilator agent, which could be either general activity or be specific for pancreas or islet blood flow, comprising administering to said subject a therapeutically effective amount of a Amylin variant.

According to other preferred embodiments of the present invention, the Amylin variants and methods of the invention are used for treatment of bone disorders where stimulation of bone growth is required, such as osteoporosis, Paget's disease, malignant deposits in bone, bone loss of malignancy, or endocrine disorders, arthritis, immobility, treating fractures, and other conditions where a hypocalcemic effect is of benefit, comprising administering to said subject a therapeutically effective amount of a Amylin variant.

According to other preferred embodiments of the present invention, the Amylin variants and methods of the invention are used for treating anorexia and related states, such as cachexia and adipose tissue deficiency, comprising administering to said subject a therapeutically effective amount of a Amylin variant.

According to other preferred embodiments of the present invention, the Amylin variants and methods of the invention are used for treating pancreatitis or relieving pain caused by pancreatitis, comprising administering to said subject a therapeutically effective amount of an Amylin variant. According to other preferred embodiments of the present invention, the Amylin variants and methods of the invention are used for treatment or prevention of pain, e.g. migraine, optionally with a narcotic analgesic or other pain relief agents, comprising administering to said subject a therapeutically effective amount of an Amylin variant.

According to other embodiments, the invention provides a pharmaceutical composition comprising as an active ingredient a plurality of polypeptides having an amino acid sequence as set forth in any one of SEQ ID NOS:70-75, 87, 91, 95, 97, 101, 103, 105, 107, or a fragment thereof According to other embodiments, the invention provides a pharmaceutical composition comprising as an active ingredient at least one polypeptide having an amino acid sequence as set forth in any one of SEQ ID NOS: 70-75, 87, 91, 95, 97, 101, 103, 105 or 107, and further comprising at least one polypeptide having an amino acid sequence as set forth in any one of SEQ ID NOS:3, 7-22, 58-61, or 41-44 corresponding to splice variants of GLP-1, OXM and preproglucagon.

According to certain embodiments, the present invention provides a method for affecting the metabolic state or treating a disorder related to metabolism in a subject, comprising administering to a subject in need thereof a therapeutically effective amount of at least one polypeptide having an amino acid sequence as set forth in any one of SEQ ID NOS:70-75, 87, 101, 103, 91, 105, 107, 95 or 97, or administering to a subject in need thereof a therapeutically effective amount of an isolated nucleic acid encoding at least one polypeptide having an amino acid sequence as set forth in any one of SEQ ID NOS:70-75, 87, 101, 103, 91, 105, 107, 95 or 97.

According to one embodiment, the method comprises administering to the subject at least two polypeptides having an amino acid sequence as set forth in any one of SEQ ID NOS:70-75, 87, 101, 103, 91, 105, 107, 95 or 97, or administering to a subject in need thereof a therapeutically effective amount of at least two isolated nucleic acids encoding at least one polypeptide having an amino acid sequence as set forth in any one of SEQ ID NOS:70-75, 87, 101, 103, 91, 105, 107, 95 or 97.

According to another embodiment, the at least one polypeptide is administered in combination with at least one polypeptide having an amino acid sequence as set forth in any one of SEQ ID NOS:3, 7-22, 58-61, 35-38 or 41-44, or further comprising administering to the subject at least one isolated nucleic acid encoding at least one polypeptide having an amino acid sequence as set forth in any one of SEQ ID NOS:3, 7-22, 58-61, 35-38 or 41-44.

According to yet another embodiment, affecting the metabolic state includes treating a metabolic regulatory imbalance and/or restoring metabolic balance in a subject and/or regulating metabolism of a subject and/or treating of a disease or a condition having a pathology related to a metabolic activity wherein a beneficial therapeutic effect is achieved by metabolic regulation and/or any mechanism for which PYY treatment is suitable.

According to one embodiment, the disease or condition is selected from obesity, diabetes mellitus, eating disorders, conditions or disorders which can be alleviated by improving lipid profile, dislipidemia, conditions or disorders which can be alleviated by reducing nutrient intake or availability, reducing degeneration of pancreatic tissue, altering the differentiated state of a pancreatic islet or cell, disease associated with altered glucose metabolism, insulin resistance, hypertension, cardiovascular diseases.

Other objects, features and advantages of the present invention will become clear from the following description and drawings.

### BRIEF DESCRIPTION OF THE DRAWINGS

FIG. 1 shows multiple alignment of amino acid sequences of PYY skipping exon 3 variant (SEQ ID NO:73) (referred as HPYY skip 2), PYY2-corrected variant (SEQ ID NO:70), the known PYY (SEQ ID NO:65) and the known PYY3 amino acid sequence (SEQ ID NO:111). The amino acid residues encoded by exon 3, skipped in the PYY skipping exon 3 variant, are shown in rectangle. The signal peptide and the amino acid residues of PYY[l-36] and PYY[3-36] are indicated.

FIG. 2 shows the variant structures of PYY2 corrected and PYY skipping exon 3.

FIG. 3A-3C shows the exon structure of the NPY family, marking the exon, which is skipped in the PYY, NPY and PPY variants of the present invention. FIG. 3A shows the known PYY exons (accession number NM_{—}004160, SEQ ID NO:115). The 81 bp-length exon marked as “exon 5” (previously known as “exon 3”), is the one skipped in the PYY skipping exon 3 variant of the present invention. FIG. 3B shows the known NPY exons (accession number NM_{—}000905, SEQ ID NO:116). The 81 bp-length exon marked as “exon 3” is the one skipped in the NPY variant of the present invention. FIG. 3C shows the known PPY exons (accession number NM_{—}002722, SEQ ID NO:117). The 72 bp-length exon marked as “exon 2” is the one skipped in the PPY variant of the present invention. In all cases, the coding sequence is underlined. As shown in FIGS. 9-11 below, the exon skipping results in creation of a similar protein variants in all three members of the PP family.

FIG. 4 is a schematic illustration showing the protein domain structure of wild-type preproPYY protein (SwissProt accession: PYY_HUMAN, SEQ ID NO:65) and the preproPYY variant skipping exon 3 of the present invention (SEQ ID NO:73). The code is as follows: signal peptide is marked with wide upward diagonal lines; pro-peptide sequence is marked with vertical lines; peptide sequence is marked with wide downward diagonal lines; and cleavage sites are marked with light upward diagonal lines.

FIG. 5 is a schematic illustration showing the protein domain structure of wild-type NPY protein (SwissProt accession: NEUY_HUMAN, SEQ ID NO:86) and the NPY variant of the present invention (SEQ ID NO:87). The code is as follows: signal peptide is marked with wide upward diagonal lines; pro-peptide sequence is marked with vertical lines; peptide sequence is marked with wide downward diagonal lines; and cleavage sites are marked with light upward diagonal lines.

FIG. 6 is a schematic illustration showing the protein domain structure of wild-type PPY protein (SwissProt accession: PAHO_HUMAN, SEQ ID NO:90) and the PPY variant of the present invention (SEQ ID NO:91). The code is as follows: signal peptide is marked with wide upward diagonal lines; pro-peptide sequence is marked with vertical lines; peptide sequence is marked with wide downward diagonal lines; and cleavage sites are marked with light upward diagonal lines.

FIG. 7 is a schematic illustration showing the protein domain structure of wild-type Amylin protein (SwissProt accession: IAPP_HUMAN, SEQ ID NO:93) and the Amylin variants 1 and 2 of the present invention (SEQ ID NO:95 and 97). The code is as follows: unique regions are marked with horizontal lines (SEQ ID NOS:98 and 99, for Amylin variant 1 and 2, respectively); signal peptide is marked with wide upward diagonal lines; pro-peptide sequence is marked with vertical lines; peptide sequence is marked with wide downward diagonal lines; and cleavage sites are marked with light upward diagonal lines

FIGS. 8A and 8B presents the amino acid sequence of the Amylin variant 1 (FIG. 8A) and Amylin variant 2 (FIG. 8B) of the present invention (SEQ ID NO:95 and 97, respectively), described in Example 4 of the Examples section which follows. The unique sequence is marked by rectangle.

FIG. 9A and 9B shows the alignment between the known PYY protein and the PYY skipping exon 3 variant of the present invention. In FIG. 9A the comparison between the known preproPYY (SEQ ID NO:65) and the preproPYY variant skipping 3 (SEQ ID NO:73) is shown. In FIG. 9B the comparison between the known PYY [1-36] (SEQ ID NO:66) and the PYY variant skipping 3 [1-42] (SEQ ID NO:74) is shown.

FIG. 10 shows the alignment between the known NPY protein and the NPY variant of the present invention. In FIG. 10A the comparison between the known preproNPY (SEQ ID NO:86) and the preproNPY variant (SEQ ID NO:87) is shown. In FIG. 10B the comparison between the mature known NPY [1-36] (SEQ ID NO:100) and the mature NPY variant [1-42] (SEQ ID NO:101) is shown.

FIG. 11 shows the alignment between the known PPY protein and the PPY variant of the present invention. In FIG. 11A the comparison between the known preproPPY (SEQ ID NO:90) and the preproPPY variant (SEQ ID NO:91) is shown. In FIG. 11B the comparison between the mature known PPY [1-36] (SEQ ID NO:104) and the mature PPY variant [1-42] (SEQ ID NO:105) is shown.

FIG. 12 shows the alignment between the known Amylin protein and the Amylin variants of the present invention. In FIG. 12A the comparison between the known Amylin (SEQ ID NO:93) and the Amylin variant 1 (SEQ ID NO:95) is shown. In FIG. 12B the comparison between the known Amylin (SEQ ID NO:93) and the Amylin variant 2 (SEQ ID NO:97) is shown.

FIG. 13 shows the results of the in-vitro stability study for the WT PYY[3-36] and the PYY variant of the invention [3-42] in aceteate buffer (20 mM pH4.0), as described in details in Example 7, in the Examples section below. FIG. 13A shows the average recovery while FIG. 13B shows the average peak purity of the WT PYY[3-36] and the PYY variant of the invention [3-42] during the 28 days of the experiment. The code is as follows: PYY[3-36] low dose is shown with rhombus line; PYY[3-36] high dose is shown with square line; PYY variant [3-42] low dose is shown with circle line; PYY variant [3-42] high dose is shown with “x” line.

FIG. 14 shows the OD values for negative control in ELISA test, as described in details in Example 8, in the Examples section below.

FIG. 15 shows the reactivity of the P3S sera, raised against the peptide derived from the unique tail of PYY variant of the invention, as described in details in Example 8, in the Examples section below.

FIG. 16 shows the reactivity of the P3J sera, raised against the peptide derived from the junction between the common region of the PYY variant with the known peptide and the unique tail of PYY variant of the invention, as described in details in Example 8, in the Examples section below.

FIG. 17 shows the OD values obtained in the ELISA test when the plate was coated with the PYY variant, as described in details in Example 8, in the Examples section below.

FIG. 18 shows the sequence of the peptide P3J (SEQ ID NO:110), used to produce PYY skipping exon 3 variant specific antibodies, as described in details in Example 8, in the Examples section below. The sequence, common to the PYY variant and the known PYY is shown in bold; the sequence unique to the PYY variant is underlined; the remaining sequence is a spacer.

FIG. 19 shows the expression of the PYY skipping exon 3 variant mRNA using PCR analysis in tissue samples as described in Example 5 in the Examples section below. Known PYY isoforms are marked as “PYY-WT intron 3 retention” (D13902; SEQ ID NO:124), and “PYY-WT”. The PYY skipping exon 3 variant mRNA was expressed in male urogenital tissues (lane 2), intestine and pancreas (lane 4) and colon (lane 7).

FIG. 20 shows the expression of the NPY variant and PPY variant mRNAs using PCR analysis in tissue samples as described in Example 5 in the Examples section below. The NPY variant mRNA was expressed in brain and pancreas. The PPY variant mRNA was expressed only in pancreas.

### DETAILED DESCRIPTION OF THE INVENTION

The present invention relates to novel splice variants of PYY, NPY, PPY and Amylin, and methods and compositions of using same. Table 1 presents a summary of the amino acid and nucleotide sequences of the splice variants of this invention, the splice variants of GLP-1, OXM and preproglucagon, described in PCT/IL2004/000952 by the present inventors, filed on Oct. 17, 2004, and hereby incorporated by reference as if fully set forth herein, and the known (wild type) preproglucagon, GLP-1, OXM, PYY, NPY, PPY and Amylin.

TABLE 1

| | | Corresponding |
|---|---|---|
| | Polypeptide | nucleotide |
| Description | SEQ ID NO: | SEQ ID NO: |
| Known preproglucagon | 1 | 2 |
| Known Homo Sapiens Glucagon, gi: 20302161 | | 57 |
| Preproglucagon splice variant | 3 | 4 |
| Known GLP-1 | 5 | 6 |
| GLP-1 splice variant 1 | 7 | 23 |
| GLP-1 splice variant 2 | 8 | 24 |
| GLP-1 splice variant 3 | 9 | 25 |
| GLP-1 splice variant 4 | 10 | 26 |
| GLP-1 splice variant 5 | 11 | 27 |
| GLP-1 splice variant 6 | 12 | 28 |
| GLP-1 splice variant 7 | 13 | 29 |
| GLP-1 splice variant 8 | 14 | 30 |
| GLP-1 splice variant 9 | 58 | 62 |
| GLP-1 splice variant 10 | 59 | 63 |
| Amidated GLP-1 splice variant 1 | 15 | |
| Amidated GLP-1 splice variant 2 | 16 | |
| Amidated GLP-1 splice variant 3 | 17 | |
| Amidated GLP-1 splice variant 4 | 18 | |
| Amidated GLP-1 splice variant 5 | 19 | |
| Amidated GLP-1 splice variant 6 | 20 | |
| Amidated GLP-1 splice variant 7 | 21 | |
| Amidated GLP-1 splice variant 8 | 22 | |
| Amidated GLP-1 splice variant 9 | 60 | |
| Amidated GLP-1 splice variant 10 | 61 | |
| Modified GLP-1 variant 5 | 64 | |
| Unique amino acids at C-terminus of GLP-1 splice | 35 | 31 |
| variant 1 | | |
| Unique amino acids at C-terminus of GLP-1 splice | 36 | 32 |
| variant 2 | | |
| Unique amino acids at C-terminus of GLP-1 splice | 37 | 33 |
| variant 3 | | |
| Unique amino acids at C-terminus of GLP-1 splice | 38 | 34 |
| variant 4 | | |
| Known OXM | 39 | 40 |
| OXM splice variant 1 | 41 | 45 |
| OXM splice variant 2 | 42 | 46 |
| OXM splice variant 3 | 43 | 47 |
| OXM splice variant 4 | 44 | 48 |
| Unique amino acids at C-terminus of OXM splice | 49 | 53 |
| variant 1 | | |
| Unique amino acids at C-terminus of OXM splice | 50 | 54 |
| variant 2 | | |
| Unique amino acids at C-terminus of OXM splice | 51 | 55 |
| variant 3 | | |
| Unique amino acids at C-terminus of OXM splice | 52 | 56 |
| variant 4 | | |
| Known preproPYY | 65 | 76 |
| Known preproPYY, CDS | | 77 |
| Known preproPYY, NM_004160.2 | | 115 |
| Known preproPYY intron 3 retention, D13902 | | 124 |
| Known PYY [1-36] | 66 | |
| Known PYY [3-36] | 67 | |
| Known | 68 | 78 |
| PreprohPYY2 | | |
| Known | | 79 |
| PreprohPYY2, CDS | | |
| Known | 69 | |
| hPYY2 [1-15] | | |
| PYY Variant 1: hPYY2 PreproPYY2 Corrected | 70 | 80 |
| PYY Variant 1: hPYY2 PreproPYY2 Corrected, | | 81 |
| CDS | | |
| PYY Variant 1: hPYY2 corrected [1-35] | 71 | |
| PYY Variant 1: hPYY2 corrected [3-35] | 72 | |
| PYY Variant 2: prepro-hPYY_skipping_exon_3 | 73 | 82 |
| (prepro-PYY skipping exon 3 variant) | | |
| PYY Variant 2: prepro-hPYY_skipping_exon_3 | | 83 |
| (prepro-PYY skipping exon 3 variant), CDs | | |
| PYY Variant 2: hPYY_skipping_exon_3 [1-42] | 74 | |
| (PYY skipping exon 3 variant [1-42]) | | |
| PYY Variant 2: hPYY_skipping_exon_3 [3-42] | 75 | |
| (PYY skipping exon 3 variant [3-42]) | | |
| Known preproNPY | 86 | 84 |
| Known preproNPY, NM_000905.2 | | 116 |
| preproNPY variant skipping exon 3 | 87 | 85 |
| Known preproPPY | 90 | 88 |
| Known preproPPY, NM_002722.2 | | 117 |
| preproPPY variant skipping exon 2 | 91 | 89 |
| Known Amylin | 93 | 92 |
| Amylin variant 1 | 95 | 94 |
| Amylin variant 2 | 97 | 96 |
| Unique amino acids of Amylin variant 1 | 98 | |
| Unique amino acids of Amylin variant 2 | 99 | |
| Known NPY [1-36] | 100 | |
| NPY variant [1-42] | 101 | |
| Known NPY [3-36] | 102 | |
| NPY variant [3-42] | 103 | |
| Known PPY [1-36] | 104 | |
| PPY variant [1-42] | 105 | |
| Known PPY [3-36] | 106 | |
| PPY variant [3-42] | 107 | |
| Known Chicken Amylin RNA | | 108 |
| P3S: PYY skipping exon 3 variant derived peptide | 109 | |
| used for Abs | | |
| P3J: PYY skipping exon 3 variant derived peptide used for | 110 | |
| Abs | | |
| Known PYY3 | 111 | |
| PYY skipping exon 3 variant unique amino acids | 112 | |
| NPY variant unique amino acids | 113 | |
| PPY variant unique amino acids | 114 | |
| PYY PCR primers, forward and reverse | | 118, 119 |
| NPY PCR primers, forward and reverse | | 120, 121 |
| PPY PCR primers, forward and reverse | | 122, 123 |

PYY Variants:

In one aspect of the present invention, the PYY variant has an amino acid sequence, which corresponds to, or is homologous to SEQ ID NOS as follows: prepro form is SEQ ID NO:73, PYY_skipping exon 3 [1-42] is SEQ ID NO:74, PYY_skipping exon 3 [3-42] is SEQ ID NO:75, corresponding to various forms of PYY skipping exon 3. According to other embodiments, the PYY variant has an amino acid sequence, which corresponds to, or is homologous to SEQ ID NOS:70 (preproPYY2 corrected), SEQ ID NOS:71 (PYY2 [1-35] corrected), or SEQ ID NOS:72 (PYY2[3-35] corrected).

According to other embodiments, the PYY variant has an isolated nucleic acid encoding for a PYY skipping exon 3 variant, having a nucleotide sequence as set forth in SEQ ID NO:82. According to other embodiments, the PYY variant has an isolated nucleic acid encoding for a PYY2 corrected variant, having a nucleotide sequence as set forth in SEQ ID NO:80.

According to other embodiments, the present invention provides a PYY skipping exon 3 variant, wherein the variant is an isolated chimeric polypeptide, comprising a first amino acid sequence being at least about 90% and preferably at least about 95% homologous to amino acids 1-63 of known preproPYY (SEQ ID NO:65), which also corresponds to amino acids 1-63 of preproPYY skipping exon 3 variant (SEQ ID NO:73), and a second amino acid sequence being at least about 85% homologous to amino acids 91-97 of known preproPYY (SEQ ID NO:65), which also corresponds to amino acids 64-70 of preproPYY skipping exon 3 variant (SEQ ID NO:73), wherein said first and said second amino acid sequences are contiguous and in sequential order.

According to other embodiments, the present invention provides a PYY skipping exon 3 variant [1-42], wherein the variant is an isolated chimeric polypeptide comprising a first amino acid sequence being at least about 90% and preferably at least about 95% homologous to amino acids 1-35 of known PYY (SEQ ID NO:66) which also corresponds to amino acids 1-35 of PYY skipping exon 3 [1-42] variarit (SEQ ID NO:74), and a second amino acid sequence being at least 70%, optionally at least 80%, preferably at least 85% homologous to a peptide having the sequence SEGPDLW (SEQ ID NO:112) corresponding to amino acids 36-42 of PYY skipping exon 3 [1-42] variant (SEQ ID NO:74), wherein said first and said second amino acid sequences are contiguous and in sequential order.

According to other embodiments, the present invention relates to bridges, tails, and/or insertions, and/or analogs, homologs and derivatives of such peptides. Such bridges, tails, and/or insertions are described in greater detail below with regard to the Examples.

As used herein a “tail” refers to a peptide sequence at the end of-an amino acid sequence that is unique to a splice variant according to the present invention. Therefore, a splice variant having such a tail may optionally be considered as a chimera, in that at least a first portion of the splice variant is typically highly homologous (often 100% identical) to a portion of the corresponding “known protein”, while at least a second portion of the variant comprises the tail.

As used herein “an edge portion” refers to a connection between two portions of a splice variant according to the present invention that were not joined in the known or known protein. An edge may optionally arise due to a join between the above “known protein” portion of a variant and the tail, for example, and/or may occur if an internal portion of the known sequence is no longer present, such that two portions of the sequence are now contiguous in the splice variant that were not contiguous in the known protein. A “bridge” may optionally be an edge portion as described above, but may also include a join between a head and a “known protein” portion of a variant, or a join between a tail and a “known protein” portion of a variant, or a join between an insertion and a “known protein” portion of a variant.

As used herein the phrase “known protein” refers to a known database provided sequence of a specific protein, including, but not limited to, SwissProt (http://ca.expasy.org/), National Center of Biotechnology Information (NCBI) (http://www.ncbi.nlm.nih.gov/), PIR (http://pir.georgetown.edu/), A Database of Human Unidentified Gene-Encoded Large Proteins [HUGE <http://www.kazusa.orjp/huge>], Nuclear Protein Database [NPDhttp://npd.hgu.mrc.ac.uk], human mitochondrial protein database (http://bioinfo.nist.gov:8080/examples/servlets/index.html), and University Protein Resource (UniProt) (htt,://www.expasv.uniprot.org/).

According to other embodiments, there is provided an isolated chimeric polypeptide comprising an edge portion of PYY skipping exon 3 variant, comprising a polypeptide having a length “n”, wherein n is at least about 10 amino acids in length, optionally at least about 20 amino acids in length, optionally at least about 30 amino acids in length, optionally at least about 40 amino acids in length and optionally at least about 50 amino acids in length, wherein at least two amino acids comprise RS, having a structure as follows (numbering according to SEQ ID NO:74): a sequence starting from any of amino acid numbers 35−x to 35 and ending at any of amino acid numbers 36+((n−2)−x), in which x varies from 0 to n−2, such that the value ((n−2)−x) is not allowed to be larger than 6.

For example, for peptides of 10 amino acids (such that n=10), the starting position could be as “early” in the sequence as amino acid number 27 if x=n−2=8 (i.e. 27=35−8), such that the peptide would end at amino acid number 36 (36+(8−8=0)). On the other hand, the peptide could start at amino acid number 33 if x=2 (i.e. 35=35−30 2), and could end at amino acid 42 (42=36+((10−2)−2))).

According to other embodiments, there is provided a bridge portion comprising a polypeptide being at least 70%, optionally at least about 80%, preferably at least about 85%, more preferably at least about 90% and most preferably at least about 95% homologous to at least one edge sequence described above.

Similarly, the bridge portion may optionally be relatively short, such as from about 4 to about 9 amino acids in length. For four amino acids, the first bridge portion would comprise the following peptides: RSEG, QRSE, RQRS. All peptides feature RS as a portion thereof. Peptides of from about five to about nine amino acids could optionally be similarly constructed.

NPY Variants:

According to other embodiments, the NPY variant has an amino acid sequence, which corresponds to, or is homologous to SEQ ID NOS as follows: prepro form is SEQ ID NO:87, NPY variant [1-42] is SEQ ID NO:101, NPY variant [3-42] is SEQ ID NO:103, corresponding to various forms of NPY variant.

According to other embodiments, the NPY variant has an isolated nucleic acid is encoding for an NPY variant, having a nucleotide sequence as set forth in SEQ ID. NO:85.

According to other embodiments, the present invention provides an NPY variant, wherein the variant is an isolated chimeric polypeptide, comprising a first amino acid sequence being at least about 90% and preferably at least about 95% homologous to amino acids 1-63 of known NPY (SEQ ID NO:86), which also corresponds to amino acids 1-63 of NPY variant (SEQ ID NO:87), and a second amino acid sequence being at least about 85% homologous to amino acids 91-97 of known NPY (SEQ ID NO:86), which also corresponds to amino acids 64-70 of NPY variant (SEQ ID NO:87), wherein said first and said second amino acid sequences are contiguous and in sequential order.

According to other embodiments the present invention provides an NPY variant [1-42], wherein the variant is an isolated chimeric polypeptide, comprising a first amino acid sequence being at least about 90% and preferably at least about 95% homologous to amino acids 1-35 of known NPY[1-36] (SEQ ID NO: 100) which also corresponds to amino acids 1-35 of NPY [1-42] variant (SEQ ID NO:101), and a second amino acid sequence being at least sequence being at least 70%, optionally at least 80%, preferably at least 85% homologous to a peptide having the sequence LEDPAMW (SEQ ID NO:113) corresponding to amino acids 36-42 of NPY [1-42] variant (SEQ ID NO:101), wherein said first and said second amino acid sequences are contiguous and in sequential order.

According to other embodiments, the present invention provides an isolated chimeric polypeptide comprising an edge portion of NPY variant, comprising a polypeptide having a length “n”, wherein n is at least about 10 amino acids in length, optionally at least about 20 amino acids in length, optionally at least about 30 amino acids in length, optionally at least about 40 amino acids in length and optionally at least about 50 amino acids in length, wherein at least two amino acids comprise RL having a structure as follows (numbering according to SEQ ID NO:87): a sequence starting from any of amino acid numbers 63−x to 63 and ending at any of amino acid numbers 64+((n−2)−x), in which x varies from 0 to n−2, such that the value ((n−2)−x) is not allowed to be larger than 6.

According to other embodiments, there is provided an isolated chimeric polypeptide comprising an edge portion of NPY variant, comprising a polypeptide having a length “n”, wherein n is at least about 10 amino acids in length, optionally at least about 20 amino acids in length, optionally at least about 30 amino acids in length, optionally at least about 40 amino acids in length and optionally at least about 50 amino acids in length, wherein at least two amino acids comprise RL, having a structure as follows (numbering according to SEQ ID NO:101): a sequence starting from any of amino acid numbers 35−x to 35 and ending at any of amino acid numbers 36+((n−2)−x), in which x varies from 0 to n−2, such that the value ((n−2)−x) is not allowed to be larger than 6.

For example, for peptides of 10 amino acids (such that n=10), the starting position could be as “early” in the sequence as amino acid number 27 if x=n−2=8 (i.e. 27=35−8), such that the peptide would end at amino acid number 36 (36+(8−8=0)). On the other hand, the peptide could start at amino acid number 33 if x=2 (i.e. 35=35−2), and could end at amino acid 42 (42=36+((10−2)−2))).

According to other embodiments, there is provided a bridge portion comprising a polypeptide being at least 70%, optionally at least about 80%, preferably at least about 85%, more preferably at least about 90% and most preferably at least about 95% homologous to at least one edge sequence described above.

Similarly, the bridge portion may optionally be relatively short, such as from about 4 to about 9 amino acids in length. For four amino acids, the first bridge portion would comprise the following peptides: RLED, QRLE, RQRL. All peptides feature RL as a portion thereof. Peptides of from about five to about nine amino acids could optionally be similarly constructed.

PPY Variants:

According to other embodiments, the PPY variant has an amino acid sequence, which corresponds to, or is homologous to SEQ ID NOS as follows: prepro form is SEQ ID NO:91, PPY variant [1-42] is SEQ ID NO:105, PPY variant [3-42] is SEQ ID NO:107, corresponding to various forms of PPY variant.

According to other embodiments, the PPY variant has an isolated nucleic acid encoding for a PPY variant, having a nucleotide sequence as set forth in SEQ ID NO:89.

According to other embodiments, the present invention provides a PPY variant, wherein the variant is an isolated chimeric polypeptide, comprising a first amino acid sequence being at least about 90% and preferably at least about 95% homologous to amino acids 1-64 of known PPY (SEQ ID NO:90), which also corresponds to amino acids 1-64 of PPY variant (SEQ ID NO:91), and a second amino acid sequence being at least about 85% to amino acids 89-95 of known PPY (SEQ ID NO:90), which also corresponds to amino acids 65-71 of NPY variant (SEQ ID NO:91), wherein said first and said second amino acid sequences are contiguous and in sequential order.

According to other embodiments, the present invention provides a PPY variant [1-42], wherein the variant is an isolated chimeric polypeptide, comprising a first amino acid sequence being at least about 90% homologous to amino acids 1-35 of known PPY (SEQ ID NO: 104) which also corresponds to amino acids 1-35 of PPY [1-42] variant (SEQ ID NO:105), and a second amino acid sequence being at least sequence being at least 70%, optionally at least 80%, preferably at least 85% homologous to a peptide having the sequence ELSPLDL (SEQ ID NO:114) corresponding to amino acids 36-42 of PPY [1-42] variant (SEQ ID NO:105), wherein said first and said second amino acid sequences are contiguous and in sequential order.

According to other embodiments, the present invention provides an isolated chimeric polypeptide comprising an edge portion of PPY variant, comprising a polypeptide having a length “n”, wherein n is at least about 10 amino acids in length, optionally at least about 20 amino acids in length, optionally at least about 30 amino acids in length, optionally at least about 40 amino acids in length and optionally at least about 50 amino acids in length, wherein at least two amino acids comprise RE having a structure as follows (numbering according to SEQ ID NO:91): a sequence starting from any of amino acid numbers 64−x to 64 and ending at any of amino acid numbers 65+((n−2)−x), in which x varies from 0 to n−2, such that the value ((n−2)−x) is not allowed to be larger than 6.

According to other embodiments, there is provided an isolated chimeric polypeptide comprising an edge portion of PPY variant, comprising a polypeptide having a length “n”, wherein n is at least about 10 amino acids in length, optionally at least about 20 amino acids in length, optionally at least about 30 amino acids in length, and optionally at least about 40 amino acids in length, wherein at least two amino acids comprise RE, having a structure as follows (numbering according to SEQ ID NO:105): a sequence starting from any of amino acid numbers 35−x to 35 and ending at any of amino acid numbers 36+((n−2)−x), in which x varies from 0 to n−2, such that the value ((n−2)−x) is not allowed to be larger than 6.

For example, for peptides of 10 amino acids (such that n=10), the starting position could be as “early” in the sequence as amino acid number 27 if x =n−2=8 (i.e. 27=35−8), such that the peptide would end at amino acid number 36 (36+(8−8=0)). On the other hand, the peptide could start at amino acid number 33 if x=2 (i.e. 35=35−2), and could end at amino acid 42 (42=36+((10−2)−2))).

According to other embodiments, there is provided a bridge portion comprising a polypeptide being at least 70%, optionally at least about 80%, preferably at least about 85%, more preferably at least about 90% and most preferably at least about 95% homologous to at least one edge sequence described above.

Similarly, the bridge portion may optionally be relatively short, such as from about 4 to about 9 amino acids in length. For four amino acids, the first bridge portion would comprise the following peptides: RELS, PREL, RPRE. All peptides feature RE as a portion thereof. Peptides of from about five to about nine amino acids could optionally be similarly constructed.

Amylin Variants:

According to other embodiments, the Amylin variant has an amino acid sequence, which corresponds to, or is homologous to SEQ ID NOS as follows: Variant 1 is SEQ ID NO:95, variant 2 is SEQ ID NO:97.

According to other embodiments, the Amylin variant has an isolated nucleic acid encoding for an Amylin variant, having a nucleotide sequence as set forth in SEQ ID NOS:94 or 96.

According to other embodiments, the present invention provides an Amylin variant 1, wherein the variant is an isolated chimeric polypeptide, comprising a first, amino acid sequence being at least about 90% and preferably at least about 95% homologous to amino acids 1-26 of known Amylin (SEQ ID NO:93), which also corresponds to amino acids 1-26 of Amylin variant 1 (SEQ. ID NO:95), an amino acid sequence being at least 70%, optionally at least 80%, preferably at least 85%, more preferably at least 90% homologous to a bridging peptide having the sequence RCLDQIPIFTVFQEN (SEQ ID NO:98), and a second amino acid sequence being at least about 90 % homologous to amino acids 28-89 of known Amylin (SEQ ID NO:93), which also corresponds to amino acids 42-103 of Amylin variant 1 (SEQ ID NO:95), wherein said first amino acid is contiguous to said bridging eptide and said second amino acid sequence is contiguous to said bridging peptide, and wherein said first amino acid, said bridging peptide and said second amino acid sequence are in sequential order.

According to other embodiments, there is provided an isolated polypeptide comprising an edge portion of Amylin variant 1 (SEQ ID NO:95), comprising an amino acid sequence being at least 70%, optionally at least about 80%, preferably at least about 85% to the sequence RCLDQIPIFTVFQEN, (SEQ ID NO:98).

According to other embodiments, there is provided an isolated chimeric polypeptide comprising an edge portion of Amylin variant 1 (SEQ ID NO:95), comprising a polypeptide having a length “n”, wherein n is at least about 10 amino acids in length, optionally at least about 20 amino acids in length, optionally at least about 30 amino acids in length, optionally at least about 40 amino acids in length and optionally at least about 50 amino acids in length, wherein at least two amino acids comprise ER having a structure as follows (numbering according to SEQ ID NO:95): a sequence starting from any of amino acid numbers 26−x to 26 and ending at any of amino acid numbers 27+((n−2)−x), in which x varies from 0 to n−2.

For example, for peptides of 10 amino acids (such that n=10), the starting position could be as “early” in the sequence as amino acid number 18 if x=n−2=8 (i.e. 18=26−8), such that the peptide would end at amino acid number 27 (27+(8−8=0)). On the other hand, the peptide could start at amino acid number 26 if x=0 (i.e. 26=26−0), and could end at amino acid 35 (35=27+(10−2)−0).

According to other embodiments, there is provided a bridge portion comprising a polypeptide being at least 70%, optionally at least about 80%, preferably at least about 85%, more preferably at least about 90% and most preferably at least about 95% homologous to at least one edge sequence described above.

Similarly, the bridge portion may optionally be relatively short, such as from about 4 to about 9 amino acids in length. For four amino acids, the first bridge portion would comprise the following peptides: ERCL, IERC, PIER. All peptides feature ER as a portion thereof Peptides of from about five to about nine amino acids could optionally be similarly constructed.

According to other embodiments, there is provided an isolated chimeric polypeptide comprising an edge portion of Amylin variant 1 (SEQ ID NO:95), comprising a polypeptide having a length “n”, wherein n is at least about 10 amino acids in length, optionally at least about 20 amino acids in length, optionally at least about 30 amino acids in length, optionally at least about 40 amino acids in length and optionally at least about 50 amino acids in length, wherein at least two amino acids comprise NH having a structure as follows (numbering according to SEQ ID NO:95): a sequence starting from any of amino acid numbers 41−x to 41 and ending at any of amino acid numbers 42+((n−2)−x), in which x varies from 0 to n−2.

For example, for peptides of 10 amino acids (such that n=10), the starting position could be as “early” in the sequence as amino acid number 33 if x=n−2=8 (i.e. 33=41−8), such that the peptide would end at amino acid number 42 (42+(8−8=0)). On the other hand, the peptide could start at amino acid number 41 if x=0 (i.e. 41=41−8), and could end at amino acid 50 (50=42+(10−2)−0).

In another embodiment, there is provided a bridge portion comprising a polypeptide being at least 70%, optionally at least about 80%, preferably at least about 85%, more preferably at least about 90% and most preferably at least about 95% homologous to at least one edge sequence described above.

Similarly, the bridge portion may optionally be relatively short, such as from about 4 to about 9 amino acids in length. For four amino acids, the first bridge portion would comprise the following peptides: NHQV, ENHQ, QENH. All peptides feature NH as a portion thereof. Peptides of from about five to about nine amino acids could optionally be similarly constructed.

According to other embodiments, there is provided an Amylin variant 2, wherein the variant is an isolated chimeric polypeptide, comprising a first amino acid sequence being at least about 90% homologous to amino acids 1-26 of known Amylin (SEQ ID NO:93), which also corresponds to amino acids 1-26 of Amylin variant 2 (SEQ ID NO:97), and a second amino acid sequence being at least 70%, optionally at least 80%, preferably at least 85%, more preferably at least 90% and most preferably at least 95% homologous to a polypeptide having the sequence RQEWIIPVLSRNILLELRGAKPEHEAGKKSKVIRWKSGNATLPHVQRSAWQIF (SEQ ID NO:99), corresponding to amino acids 27-79 of Amylin variant 2 (SEQ ID NO:97), wherein said first and said second amino acid sequences are contiguous and in sequential order.

According to other embodiments, an isolated polypeptide comprising a tail portion of Amylin variant 2 (SEQ ID NO:97), comprising an amino acid sequence being at least 70%, optionally at least about 80%, preferably at least about 85%, more preferably at least about 90% and most preferably at least about 95% homologous to the sequence

| | |
|---|---|
| RQEWIIPVLSRNILLELRGAKPEHEAGKKSKVIRWK | (SEQ ID NO:99) |
| SGNATLPHVQRSAWQIF,. | |

According to other embodiments, there is provided an isolated chimeric polypeptide comprising an edge portion of Amylin variant 2 (SEQ ID NO:97), comprising a polypeptide having a length “n”, wherein n is at least about 10 amino acids in length, optionally at least about 20 amino acids in length, optionally at least about 30 amino acids in length, optionally at least about 40 amino acids in length and optionally at least about 50 amino acids in length, wherein at least two amino acids comprise ER having a structure as follows (numbering according to SEQ ID NO:97): a sequence starting from any of amino acid numbers 26−x to 26 and ending at any of amino acid numbers 27+((n−2)−x), in which x varies from 0 to n−2.

For example, for peptides of 10 amino acids (such that n=10), the starting position could be as “early” in the sequence as amino acid number 18 if x=n−2=8 (i.e. 18=26−8), such that the peptide would end at amino acid number 27 (27+(8−8=0)). On the other hand, the peptide could start at amino acid number 26 if x=0 (i.e. 26=26−0), and could end at amino acid 35 (35=27+(10−2)−0).

In another embodiment, there is provided a bridge portion comprising a polypeptide being at least 70%, optionally at least about 80%, preferably at least about 85%, more preferably at least about 90% and most preferably at least about 95% homologous to at least one edge sequence described-above.

Similarly, the bridge portion may optionally be relatively short, such as from about 4 to about 9 amino acids in length. For four amino acids, the first bridge portion would comprise the following peptides: ERQE, IERQ, PIER. All peptides feature ER as a portion thereof. Peptides of from about five to about nine amino acids could optionally be similarly constructed.

Homology and Homologs

The term “homology”, as used herein, refers to a degree of sequence similarity in terms of shared amino acid or nucleotide sequences. There may be partial homology or complete homology (i.e., identity). For amino acid sequence homology amino acid similarity matrices (e.g. BLOSUM62, PAM70) may be utilized in different bioinformatics programs (e.g. BLAST, FASTA, MPsrch or Scanps) and may employ the use of the Smith and Waterman algorithms, and/or global/local or BLOCKS alignments for analysis, for example. Different results may be obtained when performing a particular search with a different matrix or with a different program. Degrees of homology for nucleotide sequences are based upon identity matches with penalties made for gaps or insertions required to optimize the alignment, as is well known in the art.

The terms “homology”, “homolog” or “homologous”, in any instance herein, indicate that the sequence referred to, whether an amino acid sequence, or a nucleic acid sequence, exhibits, in one embodiment at least about 70% correspondence with the indicated sequence. In another embodiment, the amino acid sequence or nucleic acid sequence exhibits at least about 72% correspondence with the indicated sequence. In another embodiment, the amino acid sequence or nucleic acid sequence exhibits at least about 75% correspondence with the indicated sequence. In another embodiment, the amino acid sequence or nucleic acid sequence exhibits at least about 80% correspondence with the indicated sequence. In another embodiment, the amino acid sequence or nucleic acid sequence exhibits at least about 82% correspondence with the. indicated sequence. In another embodiment, the amino acid sequence or nucleic acid sequence exhibits at least about 85% correspondence with the indicated sequence. In another embodiment, the amino acid sequence or nucleic acid sequence exhibits at least about 87% correspondence with the indicated sequence. In another embodiment, the amino acid sequence or nucleic acid sequence exhibits at least about 90% correspondence with the indicated sequence. In another embodiment, the amino acid sequence or nucleic acid sequence exhibits at least about 92% correspondence with the indicated sequence. In another embodiment, the amino acid sequence or nucleic acid sequence exhibits at least about 95%. or more correspondence with the indicated sequence. In another embodiment, the amino acid sequence or nucleic acid sequence exhibits at least about 97% correspondence with the indicated sequence. In another embodiment, the amino acid sequence or nucleic acid sequence exhibits at least about 99% correspondence with the indicated sequence. In another embodiment, the amino acid sequence or nucleic acid sequence exhibits 95%-100% correspondence with the indicated sequence. Similarly, as used herein, the reference to a correspondence to a particular sequence includes both direct correspondence, as well as homology to that sequence as herein defined.

In one embodiment, the term “amino acid” or “amino acids” anywhere herein includes the 20 naturally occurring amino acids. In another embodiment, the term “amino acid” or “amino acids” includes those amino acids often modified post-translationally in vivo, such as, for example, hydroxyproline, phosphoserine and phosphothreonine. In another embodiment, particularly for analogs or homologs prepared by peptide synthesis, the term “amino acid” or “amino acids” anywhere herein includes non-coded amino acids such as, but not limited to: Abu (2-aminobutyric acid), Ahx6 (aminohexanoic acid), Ape5 (aminopentanoic acid), ArgOI (argininol), bAla (b-Alanine), Bpa (4-Benzoylphenylalanine), Bip (Beta-[4-biphenyl]-alanine), Dab (diaminobutyric acid), Dap (Diaminopropionic acid), Dim (Dimethoxyphenylalanine), Dpr (Diaminopropionic acid), Hol (homoleucine), HPhe (Homophenylalanine), GABA (gamma aminobutyric acid), GlyNH2 (Aminoglycine), Nle (Norleucine), Nva (Norvaline), Om (Omithine), PheCarboxy (para carboxy Phenylalanine), PheCl (para chloro Phenylalanine), PheF (para fluoro Phenylalanine), PheMe (para methyl Phenylalanine), PheNH2 (para amino Phenylalanine), PheNO2 (para nitro Phenylalanine), Phg (Phenylglycine), Thi (Thienylalanine), 2-aminoadipic acid, hydroxylysine and isodesmosine. Certain residues may require special methods for incorporation into the peptide, and sequential, divergent or convergent synthetic approaches to the peptide sequence are useful in this invention. In another embodiment, the term “amino acid” or “amino acids” includes both D- and L-amino acids, unless a specific configuration is indicated.

Conservative substitutions of amino acids as known to those skilled in the art are within the scope of the present invention. Conservative amino acid substitutions includes replacement of one amino acid with another having the same type of functional group or side chain e.g. aliphatic, aromatic, positively charged, negatively charged. These substitutions may enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Derivatives and Modifications

In another embodiment, the splice variants (interchangeably also referred to herein as variants) described anywhere herein, which comprise this invention, include salts and derivatives thereof. Such derived peptides include, but are not limited to, derivatives of native (human and non-human) polypeptides and their fragments. As used herein “peptide” indicates a sequence of amino acids linked by peptide bonds. The term “derived” is meant to include modified amino acid sequences and glycosylation variants, and covalent modifications of a native polypeptide. Peptides can be linear, cyclic or branched and the like, which conformations can be achieved using methods well known in the art.

In one embodiment, the natural aromatic amino acids, Trp, Tyr and Phe, present in any splice variant of this invention may be substituted for a synthetic or non-natural amino acid, such as, for example, TIC, naphthylelanine (Nol), ring-methylated derivatives of Phe, halogenated derivatives of Phe or o-methyl-Tyr.

In another embodiment, the splice variants of this invention may possess modifications rendering the Variants more stable while in a body or, in another embodiment, more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. It is to be understood that each of these modifications represents a separate embodiment of this invention.

As set forth above, the peptides of the invention can be linear, cyclic or branched, and the like, which conformations can be achieved using methods well known in the art. As used herein a “cyclic” peptide refers to analogs of synthetic linear peptides that can be made by chemically converting the structures to cyclic forms.

Cyclization of linear peptides is accomplished either by forming a peptide bond between the free N-terminal and C-terminal ends (homodetic cyclopeptides) or by forming a new covalent bond between amino acid backbone and/or side chain groups with one another or with N— or C-terminal ends (heterodetic cyclopeptides). For example, disulfide bonds between cysteine residues may cyclize a peptide sequence. Bifunctional reagents can be used to provide a linkage between two or more amino acids of a peptide. Another approach for peptide cyclization was introduced by Gilon et al. (Biopolymers 31:745, 1991), who proposed backbone-to-backbone cyclization of peptides. This strategy is able to effect cyclization via the carbons or nitrogens of the peptide backbone without interfering with side chains that may be crucial for interaction with the specific receptor of a given peptide. Further disclosures by Gilon and coworkers (WO 95/33765, WO 97/09344, U.S. Pat. No. 5,723,575, U.S. Pat. No. 5,811,392, U.S. Pat. No. 5,883,293 and U.S. Pat. No. 6,265,375), provided methods for producing building units required in the synthesis of backbone cyclized peptide analogs.

Homodetic cyclopeptides have no free N— or C-termini, and thus they are not susceptible to proteolysis by exopeptidases. Cyclization of linear peptides can also modulate bioactivity by increasing or decreasing the potency of binding to the target protein (Pelton, J. T., et al., Proc. Natl. Acad. Sci., U.S.A.,.82:236-239, 1985). Linear peptides are very flexible and tend to adopt many different conformations in solution.

Cyclization acts to constrain the number of available conformations, and thus, favor the more active or inactive structures of the peptide. The immunogenicity of synthetic peptides has been correlated with the experimentally observed conformational preferences in solution (Dyson, H., et al., 1988, Annual Review of Biophysics and Biophysical Chemistry, 17:305-324). Differences in immunogenicity may be indicative of differences in binding affinity of specific antibodies for cyclic peptides.

PYY, NPY and PPY variants are susceptible to protease cleavage. In another embodiment, the PYY, NPY and PPY variants are rendered more resistant to protease cleavage. Both PYY and NPY are known to have substitution of amino acids, especially Q34>P, affecting the specificity and/or stability of the peptide (e.g. David, et al., Am J Physiol Gastrointest Liver Physiol 279: G126-G131, 2000). In one embodiment, a Glutamine amino acid (Q) is replaced with another residue, in the PYY variant and/or NPY variant, rendering the Variant more resistant to protease cleavage. In one embodiment, the PYY variant and/or the NPY variant have a Glutamine (Q) residue at position number 34 (the numbering is according to SEQ ID NOS:74 and/or 101), which is substituted with a Proline (P) residue, or any other natural or modified amino acid. In another embodiment, the PYY and/or NPY and/or PPY variants are rendered more resistant to protease cleavage through the addition of an acyl chain to native PYY or NPY or PPY. In another embodiment, the PYY and/or NPY and/or PPY variants are rendered more stable through pegylation. In another embodiment, such a substitution delays absorption of the variant. Methods for preparing such modified Variants are well known in the art and are specified, for example, in Quantitative Drug Design, C. A. Ramsden Gd., Chapter 17.2, F. Choplin Pergamon Press, 1992.

Other methods for increasing the stability and/or activity and/or tissue distribution of peptides are also described in the art. In one embodiment, the splice variants are conjugated with a suitable stabilizing peptide sequence. Methods for preparing such modified Variants are described, for example, in WO99/46283 and WO98/22577. In another embodiment, the splice variants are modified through the addition of reactive groups, which are capable of forming covalent bonds with one or more blood components in vivo or ex vivo. For example, U.S. Pat. No. 6,514,500 discloses a method of preparing such modified GLP-1 peptides. In another embodiment, the splice variants are linked to polyethylene glycol polymers. Several methods for pegylation of peptides are well known in the art, for example WO04/022004 discloses a method for generating modified GLP-1 receptor agonists comprising a GLP-1 receptor agonist is linked to a polyethylene glycol polymer having a molecular weight of greater than 30 kD. Other modifications include, but are not limited to: acetylation, ADP-ribosylation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, disulfide bond formation, formation of covalent cross-links, formylation, gamma-carboxylation, glycosylation, methylation, myristoylation, phosphorylation, prenylation, selenoylation and sulfation. (See, for instance Creighton, Posttranslational Covalent Modification of Proteins, W.H. Freeman and Company, New York B.C. Johnson, Ed., Academic Press, New York 1-12, 1993; Seifter, et al., Meth Enzymol 182:626-646, 1990; Rattan et al., Ann NY Acad Sci 663:48-62, 1992).

It is to be understood that any variant-derived peptide of the present invention may be isolated, generated synthetically, obtained via translation of sequences subjected to any mutagenesis technique, as well as obtained via protein evolution techniques, well known to those skilled in the art. Splice variants of this invention also include variations due to expression in various host-cell types, such as differences in the termini due to proteolytic removal of one or more terminal amino acids, and frameshifting variations, including, for example, differences in the termini due to different amino acids.

Nucleic Acids

The invention also provides, according to other embodiments, an isolated nucleic acid molecule encoding for a PYY variant according to the present invention, as shown with regard to SEQ ID NOS:80-83, for example.

The invention also provides, according to other embodiments, any suitable nucleic acid molecule encoding for a NPY variant according to the present invention, as shown with regard to SEQ ID NO:85, for example.

The invention also provides, according to other embodiments, any suitable nucleic acid molecule encoding for a PPY variant according to the present invention, as shown with regard to SEQ ID NO:89, for example.

The invention also provides, according to other embodiments, any suitable nucleic acid molecule encoding for Amylin variant according to the present invention, as shown with regard to SEQ ID NOS:94 and 96, for example.

Because of the redundancy in the genetic code, it is to be understood that other nucleic acid sequences encoding for the PYY and/or NPY and/or PPY and/or Amylin splice variants of this invention are considered to be part of this invention, as well. Such sequences may be derived by methods well known to one in the art, including the use of computer algorithms, such as WOBBLE.

In another embodiment, the isolated nucleic acid molecule has a sequence that is complementary thereto.

A “nucleic acid molecule” of this invention is, in one embodiment, a polymeric form of nucleotides of any length, which contain deoxyribonucleotides, ribonucleotides, and/or analogs in any combination. Nucleic acid molecules, in another embodiment, may have three-dimensional structure, and may perform, in another embodiment, any function, known or unknown. The term “nucleic acid molecule” includes, in another embodiment, double-, single-stranded, and/or triple-helical molecules. In another embodiment, any nucleic acid molecule of this invention may encompass a double stranded form, or complementary forms known, or in another embodiment, predicted to comprise the double stranded form of DNA, or, in another embodiment, RNA or, in another embodiment, a hybrid molecule.

The following are non-limiting examples of nucleic acid molecules: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracyl, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support.

Nucleic acid sequence homology may be determined for any nucleic acid sequence of this invention, by, for example, the Smith-Waterman algorithm, utilized in analyzing sequence alignment protocols, as in for example, the GAP, BESTFIT, FASTA and TFASTA programs in the Wisconsin Genetics Software Package release 7.0, Genetics Computer Group, 575 Science Dr., Madison, Wis.).

In another embodiment, nucleic acid sequence homology may be determined for any nucleic acid sequence of this invention, by hybridization to a sequence of interest, which may be effected by stringent or moderate hybridization conditions. An example of stringent hybridization is the use of a hybridization solution containing 10% dextran sulfate, 1 M NaCl, 1% SDS and 5×10⁶ cpm ³²p labeled probe, at 65° C., with a final wash solution of 0.2×SSC and 0.1% SDS and final wash at 65° C.; whereas an example of moderate hybridization would be the use of a hybridization solution containing 10% dextran sulfate, 1 M NaCl, 1% SDS and 5×10⁶ cpm ³²p labeled probe, at 65° C., with a. final wash solution of 1×SSC and 0.1% SDS and final wash at 50° C.

The nucleic acids of this invention may be in either sense or antisense orientation.

The nucleic acids of the present invention can be produced by any synthetic or recombinant process such as is well known in the art. Nucleic acids according to the invention can further be modified to alter biophysical or biological properties by means of techniques known in the art. For example, the nucleic acid can be modified to increase its stability against nucleases (e.g., “end-capping”), or to modify its lipophilicity, solubility, or binding affinity to complementary sequences.

DNA according to this invention can also be chemically synthesized by methods known in the art. For example, the DNA can be synthesized chemically from the four nucleotides in whole or in part by methods known in the art. Such methods include those described in Caruthers, Science 230(4723):281-5, 1985. DNA can also be synthesized by preparing overlapping double-stranded oligonucleotides, filling in the gaps, and ligating the ends together (see, generally, Sambrook et al., Molecular Cloning: A Laboratory Manual, 2nd Edition, Cold Spring Harbor Laboratory Press, 1989; and Glover D M and Hames B D, eds., DNA Cloning, 2d ed., Vols. 1-4, IRL Press, Oxford, 1995). DNA expressing functional homologs of the protein can be prepared from wild-type DNA by site-directed mutagenesis (see, for example, Zoller, M. J. and Smith, M., Nucleic Acids Res. 10(20):6487-500, 1982; Zoller, M. J. and Smith, M. Methods Enzymol. 100:468-500, 1983; and Zoller, M. J. and Smith, M., DNA 3(6):479-88, 1984; McPherson ed., Directed Mutagenesis. A Practical Approach, IRL Press, Oxford, 1991. The DNA obtained can be amplified by methods known in the art. One suitable method is the polymerase chain reaction (PCR) method described in Saiki R. K. et al. Science 239(4839):487-91, 1988, U.S. Pat. No. 4,683,195, and Sambrook et al., 1989 cited above.

In another embodiment, this invention provides a liposome comprising the isolated nucleic acid molecules of this invention. In another embodiment, this invention provides a vector comprising the isolated nucleic acid molecules of this invention. By “vector” what is meant is a nucleic acid construct containing a sequence of interest that has been subcloned within the vector, in this case, the nucleic acid sequence encoding the splice variants of the invention. To generate the nucleic acid constructs in the context of the present invention, the polynucleotide segments encoding sequences of interest can be ligated into commercially available expression vector systems suitable for transducing/transforming cells, including mammalian cells, and for directing the expression of recombinant products within the transduced/transformed cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter genes.

A vector according to the present invention may include an appropriate selectable marker. The vector may further include an-origin of replication, and may be a shuttle vector, which can propagate both in bacteria, such as, for example, E. coli (wherein the vector comprises an appropriate selectable marker and origin of replication) and be compatible for propagation in vertebrate cells, or integration in the genome of an organism of choice. The vector according to this aspect of the present invention can be, for example, a plasmid, a bacmid, a phagemid, a cosmid, a phage, a virus or an artificial chromosome.

In another embodiment, there is provided a host cell comprising the isolated nucleic acid molecules and/or nucleic acid vectors as described herein. The cell may be a prokaryotic or an eukaryotic cell.

Prokaryotic cells may be used, in one embodiment, to produce the recombinant splice variants of the present invention, by methods well known in the art. In another embodiment, eukaryotic cells are used to produce the recombinant splice variants of this invention. In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., and Battey, I. ed., Basic Methods in Molecular Biology, Elsevier Press, NY, 1986). Cell-free translation systems can also be employed to produce polypeptides using RNAs derived from the DNA constructs of the present invention.

A host cell strain may be chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing which cleaves a “pre-pro” form of the protein may also be important for correct insertion, folding and/or function. Different host cells such as CHO, HeLa, MDCK, 293, W138, etc. have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines, which stably express a variant product according to the present invention, may be transformed using expression vectors, which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells, which successfully express the introduced sequences. Resistant clumps of stably transformed cells can be proliferated using tissue culture techniques appropriate to the cell type.

Any number of selection systems may be used to recover transformed cell lines. These include, but are not limited to, the herpes simplex virus thymidine kinase (Wigler is M., et al., Cell 11:223-32, 1977) and adenine phosphoribosyltransferase (Lowy I., et al., Cell 22:817-23, 1980) genes, which can be employed in tk- or aprt-cells, respectively. Also, antimetabolite, antibiotic or herbicide resistance can be used as the basis for selection; for example, dhfr which confers resistance to methotrexate (Wigler M., et al., Proc. Natl. Acad. Sci. 77:3567-70, 1980); npt, which confers resistance to the aminoglycosides neomycin and G-418 (Colbere-Garapin, F. et al., J. Mol. Biol., 150:1-14, 1981) and als or pat, which confer resistance to chlorsulfuron and phosphinotricin acetyltransferase, respectively (Murry, L. E. in McGraw Hill Yearbook of Science and Technology, McGraw Hill, New York, N.Y.; pp. 191-196, 1992). Additional selectable genes have been described, for example, trpB, which allows cells to utilize indole in place of tryptophan, or hisD, which allows cells to utilize histinol in place of histidine (Hartman S. C. and R. C. Mulligan, Proc. Natl. Acad. Sci. 85:8047-51, 1988). The use of visible markers has gained popularity with such markers as anthocyanins, beta-glucuronidase and its substrate, GUS, and luciferase and its substrates, luciferin and ATP, being widely used not only to identify transformants, but also to quantify the amount of transient or stable protein expression attributable to a specific vector system (Rhodes, C. A. et. al., Methods Mol. Biol., 55:121-131, 1995).

Host cells transformed with a nucleotide sequence encoding a variant product according to the present invention may be cultured under conditions suitable for the expression and recovery of the encoded protein from cell culture. The product produced by a recombinant cell may be secreted or contained intracellularly depending on the sequence and/or the vector used. As will be understood by those of skill in the art, expression vectors containing nucleic acid sequences encoding the variant product can be designed with signal sequences, which direct secretion of the variant product through a prokaryotic or eukaryotic cell membrane.

The variant product may also be expressed as a recombinant protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp, is Seattle, Wash.). The inclusion of a protease-cleavable polypeptide linker sequence between the purification domain and the variant protein is useful to facilitate purification.

One such expression vector provides for expression of a fusion protein compromising a variant product polypeptide fused to a polyhistidine region separated by an enterokinase cleavage site. The histidine residues facilitate purification on IMIAC (immobilized metal ion affinity chromatography, as described in Porath, et al., Protein Expression and Purification, 3:263-281, 1992) while the enterokinase cleavage site provides a means for isolating PSA variant polypeptide from the fusion protein. pGEX vectors (Promega, Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

Following transformation of a suitable host strain and growth of the host strain to an appropriate cell density, the selected promoter is induced by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents, or other methods, which are well know to those skilled in the art.

The variant products can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Protein refolding steps can be used, as necessary, in completing configuration of the mature protein. Finally, high performance liquid chromatography (HPLC) can be employed for final purification steps.

In another embodiment, the cells of this invention are introduced into a host. In one embodiment, such cell transfer is as a means of cell therapy.

The basis of cell therapy is to deliver a cell expressing a particular product in a tissue wherein the endogenous cell's ability to express such a product is missing or defective. Among the approaches to accomplishing cell therapy has been the use of recombinant vectors, which have been genetically engineered to carry a desired transgene, encoding for the splice variants of this invention. For example, in one embodiment, the vector is a viral vector, which infects a desired cell. In another embodiment, viral vector integrates within host cell DNA, thereby providing a continual source of expressed product. Trangene delivery may be accomplished through a variety of gene knock in methods well known in the art.

In another embodiment the vector may be introduced into desired cells by direct DNA uptake techniques, and plasmid, linear DNA or liposome mediated transduction, receptor-mediated uptake and magnetoporation methods employing calcium-phosphate mediated and DEAE-dextran mediated methods of introduction, electroporation, liposome-mediated transfection, direct injection, and receptor-mediated uptake (for further detail see, for example, Methods in Enzymology Vol. 1-317, Academic Press; Current Protocols in Molecular Biology, Ausubel F. M. et al. (eds.) Greene Publishing Associates, 1989; and Sambrook et al., 1989 cited above, or other standard laboratory manuals).

Such constructs can also be used in somatic and/or germ cell therapy to provide for expression of the splice variants of this invention. In one embodiment, such cells may comprise stem cells or progenitor cells. In one embodiment, such stem cells may differentiate in situ, following introduction into an appropriate host, and express the splice variants of the present invention.

In another embodiment, there is provided an oligonucleotide of at least about 12 nucleotides, specifically hybridizing with an isolated nucleic acid described herein. With respect to isolated nucleic acids encoding for a PYY splice variants, the isolated nucleic acid, in one embodiment, have a nucleic acid sequence as set forth in any one of SEQ ID NOS:80-83, or a sequence homologous thereto. With respect to isolated nucleic acids encoding for a NPY splice variants, the isolated nucleic acid, in one embodiment, have a nucleic acid sequence as set forth in SEQ ID NO:85, or a sequence homologous thereto. With respect to isolated nucleic acids encoding for a PPY splice variants, the isolated nucleic acid, in one embodiment, have a nucleic acid sequence as set forth in is SEQ ID NO:89, or a sequence homologous thereto. With respect to isolated nucleic acids encoding for Amylin splice variants, the isolated nucleic acid, in one embodiment, have a nucleic acid sequence as set forth in any one of SEQ ID NOS:94, 96, or a sequence homologous thereto. In another embodiment, the oligonucleotide may hybridize with a fragment thereof. In another embodiment, the oligonucleotide is sense or antisense in orientation.

Hybridization may be conducted by any of numerous methods well known in the art, and may comprise in one embodiment, moderate conditions, or in another embodiment, under stringent conditions, or in another embodiment, under conditions therebetween. In another embodiment, this invention provides compositions comprising oligonucleotides of this invention.

In one embodiment, antisense oligonucleotides of this invention may be utilized as silencers of gene expression. Such molecules specifically bind to RNA sequences, whose expression it is desired to prevent, inhibit the translation of the RNA, thereby silencing gene expression.

In another embodiment, antisense oligonucleotides modulate gene splicing. Many genes encode pre-mRNAs containing introns that are removed by a splicing process that is directed by a complex of small nuclear ribonucleic proteins (snRNPs) called the spliceosome. Gene expression is effectively inhibited by anti-sense oligonucleotide targeting the intron/exon boundaries of splice sites because these domains direct splicing events. Antisense oligonucleotides can, in another embodiment, be designed to promote or suppress splicing at a particular site, thereby being used to enhance or limit expression of a particular splice variant of this invention.

Antisense oligonucleotides are typically synthesized in lengths of about 13-30 nucleotides. In one embodiment, the antisense oligonucleotides are chemically modified to prevent destruction by ubiquitous nucleases present in the body.

RNA oligonucleotides may, in another embodiment, be used for antisense inhibition as they form a stable RNA-RNA duplex with the target, suggesting efficient inhibition.

In another embodiment, synthetic oligonucleotides capable of hybridizing with double stranded DNA are utilized. According to this aspect of the invention, a triple helix is formed. Such oligonucleotides may prevent binding of transcription factors to the gene's promoter and therefore inhibit transcription. Alternatively, they may prevent duplex unwinding and, therefore, transcription of genes within the triple helical structure.

In another embodiment, ribozymes may be generated that serve to inactivate endogenous, mutated versions of native peptides from which the splice variants of this invention are varied, which may be a means of gene therapy, whereupon the splice variants are supplied in their stead.

In another embodiment, gene silencing small interfering RNAs (siRNAs) may be utilized to silence endogenous peptides from which the splice variants of this invention are varied, such as PYY, NPY, PPY, or Amylin. Duplexes consisting of between about 21-, and 23-nucleotide siRNA generated by ribonuclease III cleavage of longer dsRNAs, and by cleavage induced by other enzymes (e.g., “dicer” in D. melanogaster (Baulcombe, D. Nature 409: 295-6, 2001, and Caplen, N. J., et al. PNAS. 98: 9742-7, 2001)) thought to be similar to RNase III, or generated artificially, are the mediators of sequence specific mRNA degradation.

In another embodiment, aptamers are utilized to silence endogenous peptides from which the splice variants of this invention are varied. Aptamers are specifically binding oligonucleotides for non-oligonucleotide targets that generally bind nucleic acids. The use of single-stranded DNA as an appropriate material for generating aptamers is disclosed in U.S. Pat. No. 5,840,567. Use of DNA aptamers has several advantages over RNA including increased nuclease stability, in particular plasma nuclease stability, and ease of amplification by PCR or other methods. RNA generally is converted to DNA prior to amplification using reverse transcriptase, a process that is not equally efficient with all sequences, resulting in loss of some aptamers from a selected pool.

In another embodiment, methods of gene silencing, utilizing the reagents listed herein may serve to prevent or for blocking the expression of endogenous PYY, NPY, PPY or Amylin. Such methods may be utilized in diseases whereby weight gain is desired, for example, such as in the treatment of anorexia, or other wasting diseases.

The antisense compounds of this invention are useful, in another embodiment, for research and diagnostics, because these compounds hybridize to nucleic acids encoding PYY, NPY, PPY or Amylin, enabling sandwich and other assays to easily be constructed to exploit this fact. Hybridization of the antisense oligonucleotides of the invention with a nucleic acid encoding PYY, NPY, PPY or Amylin, can be detected by means known in the art. Such means may include conjugation of an enzyme to the oligonucleotide, radiolabelling of the oligonucleotide or any other suitable detection means. Kits using such detection means for detecting the level of PYY, NPY, PPY or Amylin in a sample may also be prepared.

Antibodies

According to other embodiments, there is provided an antibody specifically recognizing a PYY or NPY or PPY or Amylin variants of this invention. The antibody or antibody fragment comprises an immunoglobulin specifically recognizing a PYY or NPY or PPY or Amylin variant or a portion thereof. The term “specifically recognizing” when referring to an antibody, refers to a binding reaction that is determinative of the presence of the protein in a heterogeneous population of proteins and other biologics. Thus, under designated immunoassay conditions, the specified antibodies bind to a particular protein at least about two times the background and do not substantially bind in a significant amount to other proteins present in the sample. Thus, preferably such an antibody differentially recognizes PYY or NPY or PPY or Amylin splice variants of the present invention but do not recognize known PYY or NPY or PPY or Amylin peptides.

According to other embodiments, the antibody or antibody fragment specifically recognizes an amino acid sequence corresponding to or homologous to a PYY variant according to the present invention, as shown for example by SEQ ID NOS:70-75, or a fragment thereof comprising at least one PYY variant epitope. The term “epitope” refers to a site on an antigen to which B and/or T cells respond. B-cell epitopes can be formed both from contiguous amino acids or noncontiguous amino acids juxtaposed by tertiary folding of a protein. As used herein, the term “epitope” further relates to epitopes useful to distinguish between the splice variants of this invention and known peptides.

In still another embodiment the antibody or antibody fragment specifically recognizes an amino acid sequence corresponding to or homologous to a NPY variant according to the present invention, as shown for example by SEQ ID NOS:87, 101, 103, or a fragment thereof comprising at least one NPY variant epitope.

In still another embodiment the antibody or antibody fragment specifically recognizes an amino acid sequence corresponding to or homologous to a PPY variant according to the present invention, as shown for example by SEQ ID NOS:91, 105, 107, or a fragment thereof comprising at least one PPY variant epitope.

In still another embodiment the antibody or antibody fragment specifically recognizes an amino acid sequence corresponding to or homologous to a Amylin variant according to the present invention, as shown for example by SEQ ID NOS:95, 97, or a fragment thereof comprising at least one Amylin variant epitope.

The antibodies may be, in one embodiment, coupled to a detectable moiety, which may be an enzyme, a chromogen, a fluorogen, a radioactive or a light-emitting moiety. A substrate attached to a detectable moiety may be in contact with the enzyme-coupled antibody, which may therefore serve as a means of detection of NPY, PPY, Amylin or PYY variant in a given sample.

Antibodies specific for PYY, NPY, PPY or Amylin variants, can be produced by using purified PYY, NPY, PPY or Amylin variants for the induction of derivatized variant-specific antibodies. By induction of antibodies, it is intended not only the stimulation of an immune response by injection into animals, but analogous steps in the production of synthetic antibodies or other specific binding molecules such as screening of recombinant immunoglobulin libraries. Both monoclonal and polyclonal antibodies can be produced by procedures well known in the art.

“Antibody” refers to a polypeptide ligand that is preferably substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad-immunoglobulin variable region genes. Antibodies exist, e.g., as intact immunoglobulins or as a number of well-characterized fragments produced by digestion with various peptidases. This includes, e.g., Fab′ and F(ab)′2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

The functional fragments of antibodies, such as Fab, F(ab′)2, and Fv that are capable of binding to macrophages, are described as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′) or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boemer et al. are also available for the preparation of human monoclonal antibodies (Cole et al., Monoclonal Antibodies and Cancer Therapy, Alan R. Liss, p. 77 (1985) and Boemer et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Epitopic determinants usually consist of chemically active surface groupings of molecules such as amino acids or carbohydrate side chains and usually have specific three dimensional structural characteristics, as well as specific charge characteristics.

Optionally, a unique epitope may be created in a variant due to a change in one or more post-translational modifications, including but not limited to glycosylation and/or phosphorylation, as described below. Such a change may also cause a new epitope to be created, for example through removal of glycosylation at a particular site.

An epitope according to the present invention may also optionally comprise part or all of a unique sequence portion of a variant according to the present invention in combination with at least one other portion of the variant which is not contiguous to the unique sequence portion in the linear polypeptide itself, yet which are able to form an epitope in combination. One or more unique sequence portions may optionally combine with one or more other non-contiguous portions of the variant (including a portion which may have high homology to a portion of the known protein) to form an epitope.

Splice Variant Synthesis:

According to other embodiments, the PYY variants and/or NPY variants and/or PPY variants and/or Amylin variants of this invention are produced synthetically, by any of a number of means well known in the art. The PYY variants and/or NPY variants is and/or PPY variants and/or Amylin variants may, in one embodiment, be synthesized by standard methods of solid phase peptide chemistry, such as for example, via procedures described by Steward and Young (Stewart, J. M. and Young, J. D., Solid Phase Peptide Synthesis, 2nd Ed., Pierce Chemical Company, Rockford, Ill., 1984; J. M. Stewart and J. D. Young, Solid Phase Peptide Synthesis, W.H. Freeman Co. (San Francisco), 1963; and J. Meienhofer, Hormonal Proteins and Peptides, vol. 2, p. 46, Academic Press (New York), 1973). Solution phase synthetic procedures may be carried out, such as for example, as described in G. Schroder and K. Lupke, The Peptides, Vol. 1, Acacemic Press (New York). Ligation of smaller peptides, to produce the desired peptide, and other methods of peptide synthesis may be utilized, as will be known to one skilled in the art.

The PYY variants and/or NPY variants and/or PPY variants and/or Amylin variants of the invention may have, according to other embodiments of this invention, the same physiological activity as the PYY peptide or NPY peptide or PPY peptide or Amylin peptide from which they are varied, respectively (although perhaps at a different level). In other embodiments, the splice variants of this invention may have an opposite physiological activity from the activity featured by the original peptide from which they are varied; may have a completely different, unrelated activity to the activity of the original peptide from which they are varied; or alternatively may have no activity at all, which may lead to various diseases or pathological conditions.

According to other embodiments, the PYY variants and/or NPY variants and/or PPY variants and/or Amylin variants of this invention may differ from the original sequence in terms of their stability, clearance rate, rate of degradation, tissue and cellular distribution, ligand specification, temporal expression pattern, pattern and mechanism of up and down regulation and in other biological properties not necessarily connected to activity.

Pharmaceutical Compositions and Methods of Administration.

According to other embodiments, the present invention provides a composition comprising isolated amino acid molecules of PYY variants and/or NPY variants and/or PPY variants and/or Amylin variants polypeptides or isolated nucleic acid molecules encoding PYY variants, and/or NPY variants and/or PPY variants and/or Amylin variants, oligonucleotides specifically hybridizing with, or vectors expressing same. Compositions may include lotions, ointments, gels, creams, suppositories, drops, liquids, sprays, aerosols, powders or granules, suspensions or solutions in water or non-aqueous media, sachets, capsules or tablets. Thickeners, carriers, buffers, diluents, surface active agents, preservatives, flavorings, coloring agents, dispersing aids, emulsifiers or binders may also be included, all as well other suitable additives, all of which are well known in the art.

For example, carriers and/or diluents may include starch, mannitol, lactose, magnesium stearate, sodium saccharin, talcum, cellulose, glucose, sucrose, (or other sugars), magnesium carbonate, gelatin, oil, alcohol, detergents, emulsifiers or water (preferably sterile), each of which represents a separate embodiment of this invention. The composition may be a mixed preparation of a composition or may be a combined preparation for simultaneous, separate or sequential use (including administration). The PYY variants and/or NPY variants and/or PPY variants and/or Amylin variants may be provided as a crystalline solid, a powder, an aqueous solution, a suspension or in oil, each representing an embodiment of this invention.

The compositions may be administered in any effective, convenient manner including, for instance, administration by intravenous, intraperitoneal, intramuscular, subcutaneous, intranasal or intradermal routes among others. In therapy or as a prophylactic, the active agent may be administered to an individual as an injectable composition, for example as a sterile aqueous dispersion, preferably isotonic. Compositions for oral administration may be designed to protect the active ingredient against degradation as it passes through the alimentary tract, for example, via the inclusion of a special outer coating of the formulation on a tablet or capsule, which is resistant to degradation, or allows for time release of the contents. The composition may also be packaged as a unit dose form, for example as a tablet, capsule or ampoule, for ease of administration.

Each of the splice variants of the present invention can be administered alone or in combination with at least one additional splice variant or any other agent known to be involved in glucose control and/or metabolic regulation. As use herein, the term “in combination with” refers to co-administration of the splice variant or agent, either in a combined, single pharmaceutical composition or in separate compositions. Alternatively, the term also encompasses administering one splice variant or agent as a pre-treatment followed by the application of the additional splice variant or agent.

A suitable administration format may best be determined by a medical practitioner for each patient individually. Various pharmaceutically acceptable carriers and their formulation are described in standard formulation treatises, e.g., Remington's Pharmaceutical Sciences by E. W. Martin, Mack Publishing Co. See also Wang, Y. J. and Hanson, M. A., Journal of Parenteral Science and Technology, Technical Report No. 10, Supp. 42:2S, 1988.

Splice variants according to the present invention can be provided as parenteral compositions for e.g., injection or infusion. Preferably, they are suspended in an aqueous carrier, for example, in an isotonic buffer solution at a pH of about 3.0 to about 8.0, preferably at a pH of about 3.5 to about 7.4, 3.5 to 6.0, or 3.5 to about 5.0. Useful buffers include sodium citrate-citric acid and sodium phosphate-phosphoric acid, and sodium acetate/acetic acid buffers. A form of repository or “depot” slow release preparation may be used so that therapeutically effective amounts of the preparation are delivered into the bloodstream over many hours or days following transdermal injection or delivery.

For use by the physician, the compositions are provided in dosage unit form containing an effective amount of a splice variant according to the present invention with or without another active ingredient, e.g., a food intake-reducing, plasma glucose-lowering or plasma lipid-altering agent. Therapeutically effective amounts of a splice variant according to the present invention for use in reducing nutrient availability are those that suppress appetite at a desired level. As will be recognized by one skilled in the art, an effective amount of therapeutic agent varies with many factors including the age and weight of the patient, the patient's physical condition, the blood sugar level, the weight level to be obtained, and other factors

For administration to mammals, and particularly humans, it is expected that the physician will determine the actual dosage and duration of treatment, which is most suitable for an individual and can vary with the age, weight and response of the particular individual.

Splice Variant Protein Purification

The PYY variants and/or NPY variants and/or PPY variants and/or Amylin variants of the invention, according to other embodiments, are purified by any method as is known in the art. For example, the splice variants of the invention can be purified via column chromatography, HPLC, GLC, gel electrophoresis and immunomagnetoseparation (see for example: Strategies for Protein Purification and Characterization—A Laboratory Course Manual, CSHL Press, 1996).

Splice Variant Activity

The PYY variants of the present invention, according to other embodiments, retain PYY activity. As defined herein, to “retain PYY activity” is to have a similar level of functional activity as PYY, PYY[1-36] and/or PYY[3-36]. According to other embodiments, the PYY variants possess enhanced activity, as compared to native PYY, PYY[1-36] and/or PYY[3-36]. According to other embodiments, the PYY variants exhibit enhanced stability, or in another embodiment, diminished accessibility by peptidases, as compared to native PYY, PYY[1-36] and/or PYY[3-36].

According to other embodiments, the PPY variants of the present invention retain PPY activity. To “retain PPY activity” is to have a similar level of functional activity as PPY, PPY[1-36] and/or PPY[3-36]. According to other embodiments the PPY variants possess enhanced activity, as compared to native PPY, PPY[1-36] and/or PPY[3-36]. According to other embodiments, the PPY variants exhibit enhanced stability, or in another embodiment, diminished accessibility by peptidases, as compared to native PPY, PPY[1-36] and/or PPY[3-36].

According to other embodiments, the NPY variant of the present invention has an activity antagonistic to NPY activity. An activity that is “antagonistic to NPY activity” is to have an opposite functional activity as compared to known NPY, NPY[1-36] and/or NPY[3-36]. According to other embodiments, the NPY variants exhibit enhanced stability, or in another embodiment, diminished accessibility by peptidases, as compared to native NPY, NPY[1-36] and/or NPY[3-36].

According to other embodiments, the Amylin variants of the present invention retain Amylin activity. To “retain Amylin activity” is to have a similar level of functional activity as Amylin. According to other embodiments the Amylin variants possess enhanced activity, as compared to native Amylin. According to other embodiments the Amylin variants possess reduced activity, as compared to native Amylin, thereby diminishing the potential side effects, such as a risk of insulin-induced severe hypoglycemia, nausea and amyloid fibers. According to other embodiments, the Amylin variants exhibit enhanced stability, or in another embodiment, diminished accessibility by peptidases, as compared to native Amylin.

Splice Variants as Competitive Inhibitors

According to other embodiments, the PYY variants and/or NPY variants and/or PPY variants and/or Amylin variants are contacted with a cell in order to serve as a competitive substrate for proteases that cleave the endogenous, native protein. In one embodiment, the PYY variants and/or NPY variants and/or PPY variants and/or Amylin variants compete for digestion, thereby prolonging the circulating half-life of endogenous PYY and/or NPY and/or PPY and/or Amylin. In one embodiment, the splice variants utilized for this aspect of the invention are engineered to be highly resistant to peptidase cleavage. In another embodiment, the splice variants utilized for this aspect of the invention are engineered to specifically bind to peptidases. Such methodology is well known to one skilled in the art, and may include derivatization of particular residues, such as, for example, to remove peptidase cleavage sites, wherein the splice variant is administered at a concentration in large excess of that of the native protein, thereby “soaking up” any available peptidase, preventing cleavage of the endogenous protein.

According to other embodiments, the PYY variants and/or NPY variants and/or PPY variants serve as inhibitors for di-peptidyl peptidase 4 (DPP4), and thereby prolonged the action of GLP-1.

Splice Variants and Diabetes

The PYY variants and/or NPY variants and/or PPY variants and/or Amylin variants of the invention, according to other embodiments, normalize hyperglycemia. According to other embodiments, the PYY variants and/or NPY variants and/or PPY 15 variants and/or Amylin variants normalize hyperglycemia through glucose-dependent, insulin-dependent and insulin-independent mechanisms, and, as such, are useful as primary agents for the treatment of type 2 diabetes mellitus and as adjunctive agents for the treatment of type 1 diabetes mellitus.

PYY, and/or NPY and/or PPY and/or Amylin variants according to the present invention are also useful as primary and/or adjunctive agents for the treatment of type 1 or type 2 diabetes. With regard to type 1 diabetes, the PYY variants may have regenerative activity with regard to the insulin-producing tissues of the pancreas. For example, in type 1 diabetes, any number of therapeutic regimens can be envisioned utilizing the splice variants of this invention. In one embodiment, cell therapy via implantation of pancreatic P cells engineered to express the splice variants of this invention may be accomplished, via methods well known in the art. In another embodiment, targeted delivery of vectors expressing the splice variants of this invention may be accomplished, by methods well known to one skilled in the art.

The use of an effective amount of PYY variants and/or NPY variants and/or PPY variants and/or Amylin variants as a treatment for diabetes mellitus are, in one embodiment, more potent than native PYY or the variant PYY[3-36] and/or NPY antagonists and/or known PPY and/or PPY[3-36] and/or known Amylin and/or Amylin agonists. According to other embodiments, the PYY variants and/or PPY variants and/or Amylin variants are more stable in vivo than native PYY or PYY[3-36] and/or known PPY and/or PPY[3-36] and/or known Amylin and thus are useful as a treatment for diabetes mellitus. According to other embodiments, small amounts of the PYY variants and/or NPY variants and/or PPY variants and/or Amylin variants can be administered for effective treatment. According to other embodiments, PYY variant and/or NPY variants and/or PPY variants and/or Amylin variants activity is dependent on the glucose concentration of the blood, and thus the risk of hypoglycemic side effects are greatly reduced over the risks in using current methods of treatment.

According to other embodiments, the invention provides a method for treating diabetes in a subject comprising administering to the subject an effective amount of a PYY splice variant having an amino acid sequence as set forth in any one of SEQ ID NOS:70-75, or a derivative thereof, wherein the PYY splice variant is insulinotropic in the subject, thereby treating maturity onset diabetes mellitus in the subject.

According to other embodiments, the invention provides a method for treating diabetes in a subject comprising administering to the subject an effective amount of an isolated nucleic acid encoding a PYY splice variant, wherein the PYY splice variant has an amino acid sequence as set forth in any one of SEQ ID NOS:70-75 or a derivative thereof, and wherein the PYY splice variant is insulinotropic in the subject, thereby treating maturity onset diabetes mellifus in the subject.

According to other embodiments, the invention provides a method for enhancing the expression of insulin in a pancreatic 13-type islet cell, comprising contacting a pancreatic β-type islet cell with an effective amount of a PYY splice variant having an amino acid sequence as set forth in any one of SEQ ID NOS:70-75 or a derivative thereof, under conditions that enable insulin expression, thereby enhancing the expression of insulin in a pancreatic β-type islet cell.

According to other embodiments, the invention provides a method for enhancing the expression of insulin in a pancreatic β-type islet cell, comprising contacting a pancreatic β-type islet cell with an isolated nucleic acid encoding a PYY splice variant, wherein the PYY splice variant has an amino acid sequence as set forth in any one of SEQ ID NOS:70-75 or a derivative thereof, under conditions that enable insulin expression, thereby enhancing the expression of insulin in a pancreatic β-type islet cell.

According to other embodiments, the invention provides a method of potentiating, enhancing or restoring glucose responsivity in pancreatic islets or cells. The methods can be used as therapies for diseases caused by, or coincident with aberrant glucose metabolism. The method comprising administering to a subject in need thereof a composition comprising PYY Splice variant, having an amino acid sequence as set forth in any one of SEQ ID NOS:70-75 or a derivative thereof, in an amount sufficient to increase the glucose responsiveness of a pancreatic islet or cell in the subject.

According to other embodiments, the invention provides a method of potentiating, enhancing or restoring glucose responsivity in pancreatic islets or cells. The methods can be used as therapies for diseases caused by, or coincident with aberrant glucose metabolism. The method comprising administering to a subject in need thereof a composition comprising an isolated nucleic acid encoding a PYY splice variant, wherein the PYY splice variant has an amino acid sequence as set forth in any one of SEQ ID NOS:70-75 or a derivative thereof, in an amount sufficient to increase the glucose responsiveness of a pancreatic islet or cell in the subject.

According to other embodiments, the invention provides a method for treating diabetes in a subject comprising administering to the subject a NPY splice variant having an amino acid sequence as set forth in any one of SEQ ID NOS:87, 101, 103 or a derivative thereof, wherein the NPY splice variant is insulinotropic in the subject, thereby treating maturity onset diabetes mellitus in the subject.

According to other embodiments, the invention provides a method for treating diabetes in a subject comprising administering to the subject an isolated nucleic acid encoding a NPY splice variant, wherein the NPY splice variant has an amino acid sequence as set forth in any one of SEQ ID NOS:87, 101, 103 or a derivative thereof, and wherein the NPY splice variant is insulinotropic in the subject, thereby treating maturity onset diabetes mellitus in the subject.

According to other embodiments, the invention provides a method for enhancing the expression of insulin in a pancreatic β-type islet cell, comprising contacting a pancreatic β-type islet cell with a NPY splice variant having an amino acid sequence as set forth in any one of SEQ ID NOS:87, 101, 103 or a derivative thereof, under conditions that enable insulin expression, thereby enhancing the expression of insulin in a pancreatic β-type islet cell.

According to other embodiments, the invention provides a method for enhancing the expression of insulin in a pancreatic β-type islet cell, comprising contacting a pancreatic β-type islet cell with an isolated nucleic acid encoding a NPY splice variant, wherein the NPY splice variant has an amino acid sequence as set forth in any one of SEQ ID NOS:87, 101, 103 or a derivative thereof, under conditions that enable insulin expression, thereby enhancing the expression of insulin in a pancreatic β-type islet cell.

According to other embodiments, the invention provides a method of potentiating, enhancing or restoring glucose responsivity in pancreatic islets or cells. The methods can be used as therapies for diseases caused by, or coincident with aberrant glucose metabolism. The method comprising administering to a subject in need thereof a composition comprising an NPY Splice variant having an amino acid sequence as set forth in any one of SEQ ID NOS:87, 101, 103 or a derivative thereof, in an amount sufficient to increase the glucose responsiveness of a pancreatic islet or cell in the subject.

According to other embodiments, the invention provides a method of potentiating, enhancing or restoring glucose responsivity in pancreatic islets or cells. The methods can be used as therapies for diseases caused by, or coincident with aberrant glucose metabolism. The method comprising administering to a subject in need thereof a composition comprising an isolated nucleic acid encoding a NPY splice variant, wherein the NPY splice variant has an amino acid sequence as set forth in any one of SEQ ID NOS:87, 101, 103 or a derivative thereof, in an amount sufficient to increase the glucose responsiveness of a pancreatic islet or cell in the subject.

According to other embodiments, the invention provides a method for treating diabetes in a subject comprising administering to the subject a PPY splice variant having an amino acid sequence as set forth in any one of SEQ ID NOS:91, 105, 107 or a derivative thereof, wherein the PPY splice variant is insulinotropic in the subject, thereby treating maturity onset diabetes mellitus in the subject.

According to other embodiments, the invention provides a method for treating diabetes in a subject comprising administering to the subject an isolated nucleic acid encoding a PPY splice variant, wherein the PPY splice variant has an amino acid sequence as set forth in any one of SEQ ID NO:91, 105, 107 or a derivative thereof, and wherein the PPY splice variant is insulinotropic in the subject, thereby treating maturity onset diabetes mellitus in the subject.

According to other embodiments, the invention provides a method for enhancing the expression of insulin in a pancreatic β-type islet cell, comprising contacting a pancreatic β-type islet cell with a PPY splice variant having an amino acid sequence as set forth in any one of SEQ ID NOS:91, 105, 107 or a derivative thereof, under conditions that enable insulin expression, thereby enhancing the expression of insulin in a pancreatic β-type islet cell.

According to other embodiments, the invention provides a method for enhancing the expression of insulin in a pancreatic β-type islet cell, comprising contacting a pancreatic β-type islet cell with an isolated nucleic acid encoding a PPY splice variant, wherein the PPY splice variant has an amino acid sequence as set forth in any one of SEQ ID NOS:91, 105, 107 or a derivative thereof, under conditions that enable insulin expression, thereby enhancing the expression of insulin in a pancreatic 13-type islet cell.

According to other embodiments, the invention provides a method of potentiating, enhancing or restoring glucose responsivity in pancreatic islets or cells. The methods can be used as therapies for diseases caused by, or coincident with aberrant glucose metabolism. The method comprising administering to a subject in need thereof a composition comprising PPY Splice variant, having an amino acid sequence as set forth in any one of SEQ ID NOS:91, 105, 107 or a derivative thereof, in an amount sufficient to increase the glucose responsiveness of a pancreatic islet or cell in the subject.

According to other embodiments, the invention provides a method of potentiating, enhancing or restoring glucose responsivity in pancreatic islets or cells. The methods can be used as therapies for diseases caused by, or coincident with aberrant glucose metabolism. The method comprising administering to a subject in need thereof a composition comprising an isolated nucleic acid encoding a PPY splice variant, wherein the PPY splice variant has an amino acid sequence as set forth in any one of SEQ ID NOS:91, 105, 107 or a derivative thereof, in an amount sufficient to increase the glucose responsiveness of a pancreatic islet or cell in the subject.

According to other embodiments, the invention provides a method for treating diabetes in a subject comprising administering to the subject an Amylin splice variant having an amino acid sequence as set forth in any one of SEQ ID NOS:95, 97 or a derivative thereof, wherein the Amylin splice variant is insulinotropic in the subject, thereby treating maturity onset diabetes mellitus in the subject.

According to other embodiments, the invention provides a method for treating diabetes in a subject comprising administering to the subject an isolated nucleic acid encoding an Amylin splice variant, wherein the Amylin splice variant has an amino acid sequence as set forth in any one of SEQ ID NOS:95, 97 or a derivative thereof, and wherein the Amylin splice variant is insulinotropic in the subject, thereby treating maturity onset diabetes mellitus in the subject.

According to other embodiments, the invention provides a method for enhancing the expression of insulin in a pancreatic β-type islet cell, comprising contacting a pancreatic β-type islet cell with an Amylin splice variant having an amino acid sequence as set forth in any one of SEQ ID NOS:95, 97 or a derivative thereof, under conditions that enable insulin expression, thereby enhancing the expression of insulin in a pancreatic β-type islet cell.

According to other embodiments, the invention provides a method for enhancing the expression of insulin in a pancreatic β-type islet cell, comprising contacting a pancreatic β-type islet cell with an isolated nucleic acid encoding an Amylin splice variant, wherein the Amylin splice variant has an amino acid sequence as set forth in any one of SEQ ID NO:95, 97 or a derivative thereof, under conditions that enable insulin expression, thereby enhancing the expression of insulin in a pancreatic β-type islet cell.

According to other embodiments, the invention provides a method of potentiating, enhancing or restoring glucose responsivity in pancreatic islets or cells. The methods can be used as therapies for diseases caused by, or coincident with aberrant glucose metabolism. The method comprising administering to a subject in need thereof a composition comprising an Amylin Splice variant having an amino acid sequence as set forth in any one of SEQ ID NO:95, 97 or a derivative thereof, in an amount sufficient to increase the glucose responsiveness of a pancreatic islet or cell in the subject.

According to other embodiments, the invention provides a method of potentiating, enhancing or restoring glucose responsivity in pancreatic islets or cells. The methods can be used as therapies for diseases caused by, or coincident with aberrant glucose metabolism. The method comprising administering to a subject in need thereof a composition comprising an isolated nucleic acid encoding an Amylin splice variant, wherein the Amylin splice variant has an amino acid sequence as set forth in any one of SEQ ID NO:95, 97 or a derivative thereof, in an amount sufficient to increase the glucose responsiveness of a pancreatic islet or cell in the subject.

According to other embodiments, the invention provides a method of controlling hyperglycemia in a subject in need thereof, comprising administering to the subject a PYY splice variant having an amino acid sequence as set forth in any one of SEQ ID NOS:70-75, or a derivative thereof, wherein the PYY splice variant is insulinotropic in said subject, thereby controlling hyperglycemia.

According to other embodiments, the invention provides a method of controlling hyperglycemia in a subject in need thereof, comprising administering to the subject an isolated nucleic acid encoding a PYY splice variant, wherein the PYY splice variant has an amino acid sequence as set forth in any one of SEQ ID NOS:70-75, or a derivative thereof, and wherein the PYY splice variant is insulinotropic in said subject, thereby controlling hyperglycemia.

According to other embodiments, the invention provides a method of controlling hyperglycemia in a subject in need thereof, comprising administering to the subject a NPY splice variant having an amino acid sequence as set forth in any of SEQ ID NOS:87, 101, 103 or a derivative thereof, wherein the NPY splice variant is insulinotropic in said subject, thereby controlling hyperglycemia.

According to other embodiments, the invention provides a method of controlling hyperglycemia in a subject in need thereof, comprising administering to the subject an isolated nucleic acid encoding a NPY splice variant, wherein the NPY splice variant has an amino acid sequence as set forth in any of SEQ ID NOS:87, 101, 103 or a derivative thereof, and wherein the NPY splice variant is insulinotropic in said subject, thereby controlling hyperglycemia.

According to other embodiments, the term “contacting a cell”, refers to any exposure of a cell to a peptide, nucleic acid, or composition of this invention. Cells may, in another embodiment, be in direct contact with compounds and compositions of the invention, or, in another embodiment, exposed indirectly, through methods well described in the art. For example, cells grown in media in vitro, wherein the media is supplemented with any of the PYY variant and/or NPY variants and/or PPY variants and/or Amylin variants peptides, nucleic acids, or compositions would be an example of a method of contacting a cell, considered a part of this invention.

Another example would be oral or parenteral administration of a peptide, nucleic acid or composition, whose administration results in in vivo cellular exposure to these compounds, within specific sites within a body. Such administration is also considered as part of this invention, as part of what is meant by the phrase “contacting a cell”.

According to other embodiments, the invention provides a method for diminishing insulin resistance. Insulin resistance may be due to a decrease in binding of insulin to cell-surface receptors, or to alterations in intracellular metabolism. The first type, characterized as a decrease in insulin sensitivity, can typically be overcome by increased insulin concentration. The second type, characterized as a decrease in insulin responsiveness, cannot be overcome by large quantities of insulin. Insulin resistance following trauma can be overcome by doses of insulin that are proportional to the degree of insulin resistance, and thus is apparently caused by a decrease in insulin sensitivity.

The dose of PYY variants and/or NPY variants and/or PPY variants and/or Amylin variants effective to normalize a patient's blood glucose level depends on a number of factors, among which are included, without limitation, the patient's sex, weight and age, the severity of inability to regulate blood glucose, the underlying causes of inability to regulate blood glucose, whether glucose, or another carbohydrate source, is simultaneously administered, the route of administration and bioavailability, the persistence in the body, the formulation, and the potency.

For all indications, in preferred embodiments, a PYY variant and/or NPY variants and/or PPY variants and/or Amylin variants (alone or in combination) according to the present invention is preferably administered peripherally at a dose of about 1 microgram to about 5 mg per day in single or divided doses, or at about 0.01 micrograms/kg to about 500 micrograms/kg per dose, more preferably about 0.05 micrograms/kg to about 250 micrograms/kg, most preferably below about 50 micrograms/kg. Dosages in these ranges vary with the potency of each splice variant, of course, and are readily determined by one of skill in the art.

Also, for combined therapies using one or more PYY variants and one or more GLP-1 splice variants and/or one or more NPY variants and/or one or more PPY variants and/or one or more Amylin variants, and/or one or more OXM variants, the above dosages may optionally and preferably be adjusted. Also, for the combined therapies, the splice variants may optionally be administered in a single composition and/or dosage time, or alternatively in a plurality of separate compositions and/or dosage times.

The splice variants, as described above, may optionally be used for any of the above uses related to a metabolic condition and/or condition having a pathology related to a metabolic activity and/or mechanism for which such a treatment is suitable, alone or combination with other splice variants of the present invention as described herein, and/or in combination with at least one polypeptide selected from GLP-1, OXM and preproglucagon variants, as described in PCT/IL2004/000952, and/or in combination with other agents known to be involved in glucose control (see for example US20020141985; WO98/55144, EP0844882 and U.S. Pat. No. 6,608,029, all hereby incorporated by reference as if fully set forth herein).

Splice Variants and Post Surgery Treatment

The PYY variants and/or NPY variants and/or PPY variants and/or Amylin variants of the invention may be utilized for post surgery treatments. The splice variants may be administered from about sixteen hours to about one hour before surgery begins. The length of time before surgery when the compounds used in the present invention should be administered in order to reduce catabolic effects and insulin resistance is dependent on a number of factors. These factors are generally known to the physician of ordinary skill, and include, most importantly, whether the patient is fasted or supplied with a glucose infusion or beverage, or some other form of sustenance during the preparatory period before surgery. Other important factors include the patient's sex, weight and age, the severity of any inability to regulate blood glucose, the underlying causes of any inability to regulate blood glucose, the expected severity of the trauma caused by the surgery, the route of administration and bioavailability, the persistence in the body, the formulation, and the potency of the compound administered. A preferred time interval within which to begin administration of the splice variant used in the present invention is from about one hour to about ten hours before surgery begins. The most preferred interval to begin administration is between two hours and eight hours before surgery begins.

Insulin resistance following a particular type of surgery, elective abdominal surgery, is most profound on the first post-operative day, lasts at least five days, and may take up to three weeks to normalize Thus, the post-operative patient may be in need of administration of the splice variants used in the present invention for a period of time following the trauma of surgery that depends on factors that the physician of ordinary skill will comprehend and determine. Among these factors are whether the patient is fasted or supplied with a glucose infusion or beverage, or some other form of sustenance following surgery, and also, without limitation, the patient's sex, weight and age, the severity of any inability to regulate blood glucose, the underlying causes of any inability to regulate blood glucose, the actual severity of the trauma caused by the surgery, the route of administration and bioavailability, the persistence in the body, the formulation, and the potency of the compound administered. The preferred duration of administration of the compounds used in the present invention is not more than five days following surgery.

According to other embodiments, the invention provides a method of attenuating post-surgical catabolic changes, hormonal responses to stress and insulin resistance, comprising administering to the subject an effective amount of a PYY splice variant having an amino acid sequence as set forth in any one of SEQ ID NOS:70-75 or a derivative thereof, wherein the PYY splice variant is insulinotropic in the subject, thereby attenuating post-surgical catabolic changes, hormonal responses to stress and insulin resistance.

According to other embodiments, the invention provides a method of attenuating post-surgical catabolic changes, hormonal responses to stress and insulin resistance, comprising administering to the subject an effective amount of an isolated nucleic acid encoding a PYY splice variant, wherein the PYY splice variant has an amino acid sequence as set forth in any one of SEQ ID NOS:70-75 or a derivative thereof, and wherein the PYY splice variant is insulinotropic in the subject, thereby attenuating post-surgical catabolic changes, hormonal responses to stress and insulin resistance.

According to other embodiments, the invention provides a method of attenuating post-surgical catabolic changes, hormonal responses to stress and insulin resistance, comprising administering to the subject an effective amount of a NPY splice variant having an amino acid sequence as set forth in any of SEQ ID NOS:87, 101, 103 or a derivative thereof, wherein the NPY splice variant is insulinotropic in the subject, thereby attenuating post-surgical catabolic changes, hormonal responses to stress and insulin resistance.

According to other embodiments, the invention provides a method of attenuating post-surgical catabolic changes, hormonal responses to stress and insulin resistance, comprising administering to the subject an effective amount of an isolated nucleic acid encoding a NPY splice variant, wherein the NPY splice variant has an amino acid sequence as set forth in any of SEQ ID NO:87, 101, 103 or a derivative thereof, and wherein the NPY splice variant is insulinotropic in the subject, thereby attenuating post-surgical catabolic changes, hormonal responses to stress and insulin resistance.

According to other embodiments, the invention provides a method of attenuating post-surgical catabolic changes, hormonal responses to stress and insulin resistance, comprising administering to the subject an effective amount of a PYY splice variant and/or GLP-1 splice variant and/or NPY splice variant and/or PPY splice variant and/or Amylin splice variant, and/or OXM splice variants, or a derivative thereof, wherein the combination is insulinotropic in the subject, thereby attenuating post-surgical catabolic changes, hormonal responses to stress and insulin resistance.

According to other embodiments, the invention provides a method of attenuating post-surgical catabolic changes, hormonal responses to stress and hormonal responses to stress, comprising administering to the subject an effective amount of a PYY splice variant and/or GLP-1 splice variant and/or OXM splice variants and/or NPY splice variant and/or PPY splice variant and/or Amylin splice variant or a derivative thereof, wherein the combination is insulinotropic in the subject, thereby attenuating post-surgical catabolic changes, hormonal responses to stress and hormonal responses to stress.

Splice Variants and Obesity

According to other embodiments, the invention provides a method of reducing body weight in a subject comprising administering to the subject a PYY splice variant having an amino acid sequence as set forth in any one of SEQ ID NOS:70-75, or a derivative thereof, in an amount sufficient to cause reduction in body weight in said subject.

According to other embodiments, the invention provides a method of reducing body weight in a subject comprising administering to the subject an isolated nucleic acid encoding a PYY splice variant, wherein the PYY splice variant has an amino acid as set forth in any one of SEQ ID NOS:70-75, in an amount sufficient to cause reduction in body weight in said subject.

According to other embodiments, the invention provides a method of suppressing or reducing appetite in a subject, comprising administering to the subject a PYY splice variant having an amino acid sequence as set forth in any one of SEQ ID NOS:70-75 or a derivative thereof, in an amount sufficient to cause suppression or reduction of appetite in said subject.

According to other embodiments, the invention provides a method of suppressing or reducing appetite in a subject, comprising administering to the subject an isolated nucleic acid encoding a PYY splice variant, wherein the PYY splice variant has an amino acid sequence as set forth in any one of SEQ ID NOS:70-75, or a derivative thereof, in an amount sufficient to cause suppression or reduction of appetite in said subject.

According to other embodiments, the invention provides a method of reducing body weight in a subject comprising administering to the subject a NPY splice variant having an amino acid sequence as set forth in any one of SEQ ID NOS:87, 101, 103 or a derivative thereof, in an amount sufficient to cause reduction in body weight in said subject.

According to other embodiments, the invention provides a method of reducing body weight in a subject comprising administering to the subject an isolated nucleic acid encoding a NPY splice variant, wherein the NPY splice variant has an amino acid as set forth in any one of SEQ ID NOS:87, 101, 103 in an amount sufficient to cause reduction in body weight in said subject.

According to other embodiments, the invention provides a method of suppressing or reducing appetite in a subject, comprising administering to the subject a NPY splice variant having an amino acid sequence as set forth in any one of SEQ ID NOS:87, 101, 103 or a derivative thereof, in an amount sufficient to cause suppression or reduction of appetite in said subject.

According to other embodiments, the invention provides a method of suppressing or reducing appetite in a subject, comprising administering to the subject an isolated nucleic acid encoding a NPY splice variant, wherein the NPY splice variant has an amino acid sequence as set forth in any one of SEQ ID NOS:87, 101, 103 or a derivative thereof, in an amount sufficient to cause suppression or reduction of appetite in said subject.

According to other embodiments, the invention provides a method of reducing body weight in a subject comprising administering to the subject a PPY splice variant having an amino acid sequence as set forth in any one of SEQ ID NOS:91, 105, 107 or a derivative thereof, in an amount sufficient to cause reduction in body weight in said subject.

According to other embodiments, the invention provides a method of reducing body weight in a subject comprising administering to the subject an isolated nucleic acid encoding a PPY splice variant, wherein the PPY splice variant has an amino acid as set forth in any one of SEQ ID NOS:91, 105, 107 in an amount sufficient to cause reduction in body weight in said subject.

According to other embodiments, the invention provides a method of suppressing or reducing appetite in a subject, comprising administering to the subject a PPY splice variant having an amino acid sequence as set forth in any one of SEQ ID NOS:91, 105, 107 or a derivative thereof, in an amount sufficient to cause suppression or reduction of appetite in said subject.

According to other embodiments, the invention provides a method of suppressing or reducing appetite in a subject, comprising administering to the subject an isolated nucleic acid encoding a PPY splice variant, wherein the PPY splice variant has an amino acid sequence as set forth in any one of SEQ ID NOS:91, 105, 107 or a derivative thereof, in an amount sufficient to cause suppression or reduction of appetite in said subject.

According to other embodiments, the invention provides a method of reducing body weight in a subject comprising administering to the subject a Amylin splice variant having an amino acid sequence as set forth in any one of SEQ ID NOS:95, 97 or a derivative thereof, in an amount sufficient to cause reduction in body weight in said subject.

According to other embodiments, the invention provides a method of reducing body weight in a subject comprising administering to the subject an isolated nucleic acid encoding a Amylin splice variant, wherein the Amylin splice variant has an amino acid as set forth in any one of SEQ ID NOS:95, 97 in an amount sufficient to cause reduction in body weight in said subject.

According to other embodiments, the invention provides a method of suppressing or reducing appetite in a subject, comprising administering to the subject a Amylin splice variant having an amino acid sequence as set forth in any one of SEQ ID NOS:95, 97 or a derivative thereof, in an amount sufficient to cause suppression or reduction of appetite in said subject.

According to other embodiments, the invention provides a method of suppressing or reducing appetite in a subject, comprising administering to the subject an isolated nucleic acid encoding a Amylin splice variant, wherein the Amylin splice variant has an amino acid sequence as set forth in any one of SEQ ID NOS:95, 97 or a derivative thereof, in an amount sufficient to cause suppression or reduction of appetite in said subject.

According to other embodiments, the invention provides a method of suppressing or reducing appetite in a subject, comprising administering to a subject at least one polypeptide selected from a PYY variant, a PPY variant, a NPY variant, and an Amylin variant, and optionally further comprising administering to the subject at least one polypeptide selected from GLP-1 splice variants, OXM splice variants and a Preproglucagon splice variant, wherein the combination is in an amount sufficient to cause suppression or reduction of appetite in said subject.

According to other embodiments, a PYY variant and/or a PPY variant and/or an NPY variant and/or an Amylin variant, may be supplied in a composition suitable for oral consumption, and may be utilized as a prophylactic treatment to prevent excess weight gain. According to other embodiments, administration of the PYY variant and/or a PPY variant and/or an NPY variant and/or an Amylin variant, serves as a therapeutic for reducing excess weight. Such a reduction may be administered to clinically obese individuals, to those that are overweight, and for cosmetic weight problems. The dosage of the PYY variant and/or a PPY variant and/or an NPY variant and/or an Amylin variant, are ultimately determined by the attending physician and take into consideration such factors as the PYY variant and/or a PPY variant and/or an NPY variant and/or an Amylin variant, being used, animal type, age, weight, severity of symptoms and/or severity of treatment to be applied, method of administration of the medicament, adverse reaction and/or contra indications. Specific defined dosage ranges can be determined by standard designed clinical trials with patient progress and recovery being fully monitored. Additional parameters may include timing of treatment, in terms of meal intake, and adjunctive therapies including combination therapy with special diets monitoring caloric intake, in one embodiment, or in another embodiment, in conjunction with corrective surgeries. In another embodiment, such treatment is to accompany an exercise regimen as well.

According to other embodiments, the invention provides a method of suppressing or reducing appetite in a subject, comprising administering to the subject an isolated nucleic acid encoding a PYY variant and/or a PPY variant and/or an NPY variant and/or an Amylin variant, or a derivative thereof, in an amount sufficient to cause suppression or reduction of appetite in said subject.

Splice Variants and Cardiovascular Disease

Obesity is a strong risk factor for cardiovascular disease, hypertension, atherosclerosis, congestive heart failure and stroke. According to other embodiments, the variants of the present invention are used for pharmaceutical treatment of blood pressure, cardiovascular response, and circadian rhythm.

NPY is known to decrease cardiac contractility (inotropy). Under many circumstances in which inotropy is decreased, diseases of life-threatening importance, e.g. congestive heart failure and cardiogenic shock, are associated with probable increased release of NPY into the blood. Balancing the NPY levels, preferably using NPY antagonists, may be beneficial in these disease states. NPY has also been reported to produce coronary artery vasoconstriction and thereby may decrease myocardial blood flow resulting in myocardial ischemia. Such a circumstance can result in angina pectoris or, under more severe circumstances, may result in myocardial infarction and death. According to other embodiments, the NPY variants of the present invention are useful in treatment of such problems.

According to other embodiments, the invention provides a method of treating cardiovascular disorders, as well as a method of reducing mortality and morbidity after myocardial infarction in a subject, comprising administering to the subject a PYY splice variant having an amino acid sequence as set forth in any one of SEQ ID NOS:70-75 or a derivative thereof, wherein the PYY splice variant is at a dosage effective to normalize blood glucose, thereby treating cardiovascular disorders, reducing mortality and morbidity after myocardial infarction in the subject.

According to other embodiments, the invention provides a method of treating cardiovascular disorders, as well as a method of reducing mortality and morbidity after myocardial infarction in a subject, comprising administering to the subject an isolated nucleic acid encoding a PYY splice variant, wherein the PYY splice variant has an amino acid sequence as set forth in any one of SEQ ID NOS:70-75 or a derivative thereof, and wherein the PYY splice variant is at a dosage effective to normalize blood glucose, thereby treating cardiovascular disorders, reducing mortality and morbidity after myocardial infarction in the subject.

According to other embodiments, this invention provides a method of treating cardiovascular disorders, as well as a method of reducing mortality and morbidity after myocardial infarction in a subject, comprising administering to the subject a NPY splice variant having an amino acid sequence as set forth in any one of SEQ ID NOS:87, 101, 103 or a derivative thereof, wherein the NPY splice variant is at a dosage effective to normalize blood glucose, thereby treating cardiovascular disorders, reducing mortality and morbidity after myocardial infarction in the subject.

According to other embodiments, the invention provides a method of treating cardiovascular disorders, as well as a method of reducing mortality and morbidity after myocardial infarction in a subject, comprising administering to the subject an isolated nucleic acid encoding a NPY splice variant, wherein the NPY splice variant has an amino acid sequence as set forth in any one of SEQ ID NOS:87, 101, 103 or a derivative thereof, and wherein the NPY splice variant is at a dosage effective to normalize blood glucose, thereby treating cardiovascular disorders, reducing mortality and morbidity after myocardial infarction in the subject.

According to other embodiments, the invention provides a method of treating cardiovascular disorders, as well as a method of reducing mortality and morbidity after myocardial infarction in a subject, comprising administering to the subject a PPY splice variant having an amino acid sequence as set forth in any one of SEQ ID NOS:91, 105, 107 or a derivative thereof, wherein the PPY splice variant is at a dosage effective to normalize blood glucose, thereby treating cardiovascular disorders, reducing mortality and morbidity after myocardial infarction in the subject.

According to other embodiments, the invention provides a method of treating cardiovascular disorders, as well as a method of reducing mortality and morbidity after myocardial infarction in a subject, comprising administering to the subject an isolated nucleic acid encoding a PPY splice variant, wherein the PPY splice variant has an amino acid sequence as set forth in any one of SEQ ID NOS:91, 105, 107 or a derivative thereof, and wherein the PPY splice variant is at a dosage effective to normalize blood glucose, thereby treating cardiovascular disorders, reducing mortality and morbidity after myocardial infarction in the subject.

According to other embodiments, the invention provides a method of treating cardiovascular disorders, as well as a method of reducing mortality and morbidity after myocardial infarction in a subject, comprising administering to the subject an Amylin splice variant having an amino acid sequence as set forth in any one of SEQ ID NOS:95, 97 or a derivative thereof, wherein the Amylin splice variant is at a dosage effective to normalize blood glucose, thereby treating cardiovascular disorders, reducing mortality and morbidity after myocardial infarction in the subject.

According to other embodiments, the invention provides a method of treating cardiovascular disorders, as well as a method of reducing mortality and morbidity after myocardial infarction in a subject, comprising administering to the subject an isolated nucleic acid encoding a Amylin splice variant, wherein the Amylin splice variant has an amino acid sequence as set forth in any one of SEQ ID NOS:95, 97 or a derivative thereof, and wherein the Amylin splice variant is at a dosage effective to normalize blood glucose, thereby treating cardiovascular disorders, reducing mortality and morbidity after myocardial infarction in the subject.

According to other embodiments, the invention provides a method of treating cardiovascular disorders, as well as a method of reducing mortality and morbidity after myocardial infarction in a subject, comprising administering to the subject a PYY splice variant and/or GLP-1 splice variants and/or OXM splice variants and/or NPY splice variant and/or PPY splice variant and/or Amylin splice variant, or a derivative thereof, wherein the combination is at a dosage effective to normalize blood glucose, thereby treating cardiovascular disorders, reducing mortality and morbidity after myocardial infarction in the subject.

Splice Variants and Hypertension

According to other embodiments, the invention provides a method of controlling hypertension and or dyslipidemia in a subject, comprising administering to a patient in need thereof a PYY splice variant having an amino acid sequence as set forth in any one of SEQ ID NOS:70-75 or a derivative thereof, wherein the PYY splice variant reduces nutrient availability in the patient, thereby controlling hypertension and/or dyslipidemia, by a method as described for example in US20050009748, herein incorporated by reference.

According to other embodiment, the invention provides a method of controlling hypertension and or dyslipidemia in a subject, comprising administering to a subject an isolated nucleic acid encoding a PYY splice variant, wherein the PYY splice variant has an amino acid sequence as set forth in any one of SEQ ID NOS:70-75, or a derivative thereof, and wherein the PYY splice variant PYY splice variant reduces nutrient availability in the patient, thereby controlling hypertension and/or dyslipidemia.

According to other embodiments, the invention provides a method of controlling hypertension and or dyslipidemia in a subject, comprising administering to a patient in need thereof a NPY splice variant having an amino acid sequence as set forth in any of SEQ ID NOS:87, 101, 103 or a derivative thereof, wherein the NPY splice variant reduces nutrient availability in the patient, thereby controlling hypertension and/or dyslipidemia, by a method as described for example in US20050009748, herein incorporated by reference.

According to other embodiments, the invention provides a method of controlling hypertension and or dyslipidemia in a subject, comprising administering to a subject an isolated nucleic acid encoding a NPY splice variant, wherein the NPY splice variant has an amino acid sequence as set forth in any of SEQ ID NOS:87,101, 103 or a derivative thereof, and wherein the NPY splice variant NPY splice variant reduces nutrient availability in the patient, thereby controlling hypertension and/or dyslipidemia.

According to other embodiments, the invention provides a method of controlling hypertension in a subject, comprising administering to a subject a NPY splice variant having an amino acid sequence as set forth in any of SEQ ID NOS:87, 101,103 or a derivative thereof, wherein the NPY splice variant is vasodilating in the subject, thereby controlling hypertension.

According to other embodiments, the invention provides a method of controlling hypertension in a subject, comprising administering to a subject an isolated nucleic acid encoding a NPY splice variant, wherein the NPY splice variant has an amino acid sequence as set forth in any of SEQ ID NOS:87, 101, 103 or a derivative thereof, and wherein the NPY splice variant is vasodilating in the subject, thereby controlling hypertension.

According to other embodiments, the invention provides a method of controlling hypertension and or dyslipidemia in a subject, comprising administering to a patient in need thereof a PPY splice variant having an amino acid sequence as set forth in any one of SEQ ID NOS:91, 105, 107 or a derivative thereof, wherein the PPY splice variant reduces nutrient availability in the patient, thereby controlling hypertension and/or dyslipidemia.

According to other embodiments, the invention provides a method of controlling hypertension and or dyslipidemia in a subject, comprising administering to a subject an isolated nucleic acid encoding a PPY splice variant, wherein the PPY splice variant has an amino acid sequence as set forth in any one of SEQ ID NOS:91, 105, 107, or a derivative thereof, and wherein the PPY splice variant PPY splice variant reduces nutrient availability in the patient, thereby controlling hypertension and/or dyslipidemia.

According to other embodiments, the invention provides a method of controlling hypertension and or dyslipidemia in a subject, comprising administering to a patient in need thereof a Amylin splice variant having an amino acid sequence as set forth in any of SEQ ID NOS:95, 97 or a derivative thereof, wherein the Amylin splice variant reduces nutrient availability in the patient, thereby controlling hypertension and/or dyslipidemia.

According to other embodiments, the invention provides a method of controlling hypertension and or dyslipidemia in a subject, comprising administering to a subject an isolated nucleic acid encoding a Amylin splice variant, wherein the Amylin splice variant has an amino acid sequence as set forth in any of SEQ ID NOS:95, 97 or a derivative thereof, and wherein the Amylin splice variant Amylin splice variant reduces nutrient availability in the patient, thereby controlling hypertension and/or dyslipidemia.

According to other embodiments, the invention provides a method of controlling hypertension in a subject, comprising administering to a subject a Amylin splice variant having an amino acid sequence as set forth in any of SEQ ID NOS:95, 97 or a derivative thereof, wherein the Amylin splice variant is vasodilating in the subject, which could be either general activity or be specific for pancreas or islet blood flow, comprising administering to said subject a therapeutically effective amount of a Amylin variant, thereby controlling hypertension. For the method, see for example EP0289287, hereby incorporated by reference as if fully set forth herein.

According to other embodiments, the invention provides a method of controlling hypertension in a subject, comprising administering to a subject an isolated nucleic acid encoding a Amylin splice variant, wherein the Amylin splice variant has an amino acid sequence as set forth in any of SEQ ID NOS:95, 97 or a derivative thereof, and wherein the Amylin splice variant is vasodilating in the subject, which could be either general activity or be specific for pancreas or islet blood flow, comprising administering to said subject a therapeutically effective amount of a Amylin variant, thereby controlling hypertension. In another embodiment, this invention provides a method of treating hypertension conditions in a subject, comprising administering to the subject a PYY splice variant and/or GLP-1 splice variants and/or OXM splice variants and/or NPY splice variant and/or PPY splice variant and/or Amylin splice variant, or a derivative thereof, wherein the combination is at a dosage effective to normalize the blood pressure in the subject.

Splice Variants and Anorexia and Related States

According to other embodiments of the present invention, the Amylin variants and methods of the invention are used for treating anorexia and related states, such as cachexia and adipose tissue deficiency, comprising administering to said subject a therapeutically effective amount of a Amylin variant, alone or in combination with other agents involved in metabolic regulation. An example of potential method of treating anorexia and related states is given in EP0586589, hereby incorporated by reference as if fully set forth herein. EP0586589 teaches that a patient suffering from anorexia may have fasting plasma amylin and insulin concentrations below the normal range, and in fact near the range measured by type I diabetics. Patients suffering from cachexia or receiving parenteral nutrition (i.e., nutrition except oral nutrition, e.g., intravenous) have reduced amylin and/or insulin levels, and therefore can be administered amylin with or without insulin. Such administration preferably increases adipose tissue in such patients and thus is of significant benefit.

According to other embodiments, the variants of the present invention are used for weight gain. See for example WO 96/22783, U.S. Pat. No. 5,912,227, which are hereby incorporated by reference as if fully set forth herein.

Splice Variants and Inflammatory Conditions, Bowel Conditions, Gastric Ulceration, Gastritis, Pancreatitis and Pain

According to other embodiments, the invention provides a method for preventing and/or treating inflammatory conditions in a subject, such as cutaneous, internal and/or neurogenic inflammation, including treatment of acute or chronic/persistent inflammation. The method comprising administering to a subject an antagonistic NPY splice variant having an amino acid sequence as set forth in any one of SEQ ID NOS:87, 101, 103 or a derivative thereof, wherein the NPY splice variant is anti-inflammatory in the subject.

According to other embodiments, the invention provides a method for preventing and/or treating inflammatory conditions in a subject, such as cutaneous, internal and/or neurogenic inflammation, including treatment of acute or chronic/persistent inflammation. The method comprising administering to a subject an isolated nucleic acid encoding a NPY splice variant, wherein the NPY splice variant has an amino acid sequence as set forth in any one of SEQ ID NOS:87, 101, 103 or a derivative thereof, and wherein the NPY splice variant is anti-inflammatory in the subject.

According to other embodiments, the invention provides a method of treating a bowel condition such as inflammatory bowel disease (e.g. ulcerative colitis and Crohn's disease), bowel atrophy, loss of bowel mucosa and loss of bowel mucosal function in a subject, comprising administering to a patient in need thereof a PYY splice variant having an amino acid sequence as set forth in any one of SEQ ID NOS:70-75, or a derivative thereof, by a method as described for example in WO03/105763, herein incorporated by reference.

According to other embodiments, the invention provides a method of treating a bowel condition such as inflammatory bowel disease (e.g. ulcerative colitis and Crohn's disease), bowel atrophy, loss of bowel mucosa and loss of bowel mucosal function in a subject, comprising administering to a subject an isolated nucleic acid encoding a PYY splice variant, wherein the PYY splice variant has an amino acid sequence as set forth in any one of SEQ ID NOS:70-75, or a derivative thereof.

According to other embodiments, the invention provides a method of treating or preventing gastritis or gastric ulceration in a subject, comprising administering to a subject therapeutically effective amount of an Amylin splice variant having an amino acid sequence as set forth in any of SEQ ID NOS:95, 97 or a derivative thereof. For the method, see for example EP0981360, hereby incorporated by reference as if fully set forth herein.

According to other embodiments, the invention provides a method of treating or preventing gastritis or gastric ulceration in a subject, comprising administering to a subject an isolated nucleic acid encoding a Amylin splice variant, wherein the Amylin splice variant has an amino acid sequence as set forth in any of SEQ ID NOS:95, 97 or a derivative thereof.

According to other embodiments, the invention provides a method of treating-or preventing pancreatitis or relieving pain caused by pancreatitis in a subject, comprising administering to a subject therapeutically effective amount of an Amylin splice variant having an amino acid sequence as set forth in any of SEQ ID NOS:95, 97 or a derivative thereof. For the method, see for example WO04/037168, hereby incorporated by reference as if fully set forth herein.

According to other embodiments, the invention provides a method of treating or preventing pancreatitis or relieving pain caused by pancreatitis in a subject, comprising administering to a subject an isolated nucleic acid encoding a Amylin splice variant, wherein the Amylin splice variant has an amino acid sequence as set forth in any of SEQ ID NOS:95, 97 or a derivative thereof.

According to other embodiments, the invention provides a method of treating or preventing of pain, e.g. migraine, optionally with a narcotic analgesic or other pain relief agents, comprising administering to a subject therapeutically effective amount of an Amylin splice variant having an amino acid sequence as set forth in any of SEQ ID NOS:95, 97 or a derivative thereof. For the method, see for example U.S. Pat. No. 5,677,279, hereby incorporated by reference as if fully set forth herein.

According to other embodiments, the present invention provides methods for treatment of pancreatic cells and/or tissues, as described for example in WO00/47219 to Ontogeny Inc; treatment of pancreatic tumors (WO96/14854, U.S. Pat. No. 5,574,010); induction of cellular differentiation (WO04/011621); and treatment of a bowel condition (WO3/105763); all of which are hereby incorporated by reference as if fully set forth herein.

Splice Variants and Angiogenesis

NPY is a potent angiogenic factor with known mitogenic action on smooth muscle tissue and vascular growth promoting properties, that has promising potential for the revascularization of ischemic tissue. Angiogenesis is involved in a variety of human diseases. The NPY system and Y2 receptor has been shown to play a role in the regulation of the formation of blood vessels and to be active during the development of retinopathy (Zukowska-Grojec Z. et. al.1998 (6); Lee E W, et al. 2003(16); Ekstrand A J et al. 2003(17)). Thus, identification of agents blocking the NPY mediated action thorough Y2 receptor has potential applications in the treatment of a variety of human diseases. WO04/002535, herein fully incorporated by reference, discloses a method of using NPY antagonists for treating or preventing a disease or disorder related to excessive formation of vascular tissue or blood vessels in a patient.

According to other embodiments, the invention provides a method for treating or preventing disorders related to angiogenesis, excessive formation of vascular tissue or blood vessels, including neovascular glaucoma, retinopathy, nephropathy, a cardiovascular disease or a cancerous disease. The method comprising administering to a subject an antagonistic NPY splice variant having an amino acid sequence as set forth in any one of SEQ ID NOS:87, 101, 103 or a derivative thereof, or an isolated nucleic acid encoding a NPY splice variant, wherein the NPY splice variant has an amino acid sequence as set forth in any one of SEQ ID NO:87, 101, 103 or a derivative thereof, wherein the NPY splice variant is anti-angiogenic in the subject.

Splice Variants and Bone Disorders

According to other embodiments, the invention provides a method of treating or is preventing bone disorders where stimulation of bone growth is required, such as osteoporosis, Paget's disease, malignant deposits in bone, bone loss of malignancy, or endocrine disorders, arthritis, immobility, treating fractures, and other conditions where a hypocalcemic effect is of benefit, comprising administering to a subject therapeutically effective amount of an Amylin splice variant having an amino acid sequence as set forth in any of SEQ ID NOS:95, 97 or a derivative thereof. See for example U.S. Pat. No. 5,922,677; U.S. Pat. No. 6,821,954 and EP0408284, all hereby incorporated by reference as if fully set forth herein.

According to other embodiments, the invention provides a method of treating or preventing bone disorders where stimulation of bone growth is required, such as osteoporosis, Paget's disease, malignant deposits in bone, bone loss of malignancy, or endocrine disorders, arthritis, immobility, treating fractures, and other conditions where a hypocalcemic effect is of benefit, comprising administering to a subject an isolated nucleic acid encoding a Amylin splice variant, wherein the Amylin splice variant has an amino acid sequence as set forth in any of SEQ ID NOS:95, 97 or a derivative thereof.

Splice Variants and Male Erectile Dysfunction

WO02/47670, herein fully incorporated by reference, provides an example of use of an inhibitor of a NPY for: the treatment of male sexual dysfunction, in particular male erectile dysfunction (MED). WO02/47670 discloses the method to selectively treat a male suffering from sexual dysfunction, in particular MED, with use of a neuropeptide Y inhibitor, preferably a NPY Y1 receptor inhibitor, which when in use is selective, or highly selective, for NPY or NPY Y1 receptors associated with male genitalia, without peripheral side effects. WO02/47670 demonstrates that inhibition of NPY, preferably NPY Y1, significantly enhances the nerve-stimulated erectile process.

According to other embodiments, the invention provides a method for preventing and/or treating of male erectile dysfunction, comprising administering to a subject an antagonistic NPY splice variant having an amino acid sequence as set forth in any one of SEQ ID NOS:87, 101, 103 or a derivative thereof, or an isolated nucleic acid encoding a NPY splice variant, wherein the NPY splice variant has an amino acid sequence as set forth in any one of SEQ ID NOS:87, 101, 103 or a derivative thereof. According to other preferred embodiments, the above male erectile dysfunction is in a subject suffering from diabetes mellitus.

Monitoring the Level of Administered Splice Variants

PYY variant and/or PPY variant and/or NPY variant and/or Amylin variant activity and/or in vivo concentration may be measured, in one embodiment, by assaying blood drawn from a subject administered the splice variants. Blood drawn from the host at different times, enables the determination of circulating levels of PYY variant and/or PPY variant and/or NPY variant and/or Amylin variant, and provides a means of assessing therapeutic dosage and administration times.

According to other embodiments, the splice variants may also be monitored for their insulinotropic activity, or via HPLC-MS. According to other embodiments, the splice variants may be monitored by ELISA or RIA. In another embodiment, the levels of a splice variant may be compared to that of the native protein, for diagnostic purposes, or in another embodiment, for monitoring circulating levels, or in other embodiments, for determining formulation efficacy, half-life, perfusion, and other parameters, which relate to the methods of this invention.

The insulinotropic property of splice variants may be determined, in one embodiment by providing them to animal cells, or, in another embodiment, via injection into animals and monitoring the release of immunoreactive insulin (IRI) into the media or circulatory system of the animal, respectively. The presence of IRI is detected through the use of a radioimmunoassay, which can specifically detect insulin. Insulinotropic activity may also be determined, in other embodiments, via ELISA, Western blot analysis, HPLC and other methods well known in the art.

An example of a radioimmunoassay method for insulin detection is described by Albano, J. D. M., et al., (Acta Endocrinol. 70:487-509, 1972). In this assay, a phosphate/albumin buffer with a pH of 7.4 is employed. The incubation is prepared with the consecutive condition of 500 μl of phosphate buffer, 50 μl of perfusate sample or rat insulin standard in perfusate, 100 μl of anti-insulin antiserum (Wellcome Laboratories; 1:40,000 dilution), and 100 μl of [¹²⁵I] insulin, giving a total volume of 750 μl in a 10×75-mm disposable glass tube. After incubation for 2-3 days at 4° C., free insulin is is separated from antibody-bound insulin by charcoal separation. The assay sensitivity is generally 1-2 μl U/ml. In order to measure the release of IRI into the cell culture medium of cells grown in tissue culture, one preferably incorporates radioactive label into proinsulin. Any radioactive label capable of labeling a polypeptide can be used, such as, for example, ³H leucine used to obtain labeling of proinsulin. Labeling can be done for any period of time sufficient to permit the formation of a detectably labeled pool of proinsulin molecules, with cells incubated in the presence of radioactive label for, for example, a 60-minute time period. Any cell line capable of expressing insulin can be used for determining whether a splice variant has an insulinotropic effect, such as, for example, a rat insulinoma cell line, RIN-38.

The insulinotropic property of the splice variants can also be determined by pancreatic infusion, such as via a slight modification of the method of Penhos, J. C., et al. (Diabetes 18:733-738, 1969). In accordance with such a method, fasted rats (preferably male Charles River strain albino rats), weighing 350-600 g, are anesthetized with an intraperitoneal injection of Amytal Sodium (Eli Lilly and Co., 160 ng/kg). Renal, adrenal, gastric, and lower colonic blood vessels are ligated. The entire intestine is resected except for about four cm of duodenum and the descending colon and rectum. Therefore, only a small part of the intestine is perfused, thus minimizing possible interference by enteric substances with insulinotropic immunoreactivity. The perfusate may be a modified Krebs-Ringer bicarbonate buffer with 4% dextran T70 and 0.2% bovine serum albumin (fraction V), and may be bubbled with 95% O₂ and 5% CO₂. A nonpulsatile flow, four-channel roller-bearing pump (Buchler polystatic, Buchler Instruments Division, Nuclear-Chicago Corp.) is preferably used, and a switch from one perfusate source to another is preferably accomplished by switching a three-way stopcock. The manner in which perfusion is performed, modified, and analyzed, may be, for example, as described by Weir, G. C., et al., (J. Clin. Investigat. 54:1403-1412, 1974).

HPLC coupled with mass spectrometry (MS) may also be utilized to assay for the presence of PYY variant and/or PPY variant and/or NPY variant and/or Amylin variant, as is well known to the skilled artisan. Two mobile phases are utilized: 0.1% TFA/water and 0.1% TFA/acetonitrile. Column temperatures can be varied as well as gradient conditions.

According to other embodiments, the invention provides a method for detecting PYY variant and/or PPY variant and/or NPY variant and/or Amylin variant in a biological sample, comprising the steps of: contacting the biological sample with an antibody specifically recognizing a PYY variant and/or PPY variant and/or NPY variant and/or Amylin variant polypeptide under conditions facilitating detection of antibody recognition of the PYY variant and/or PPY variant and/or NPY variant and/or Amylin variant epitope, thereby determining the presence of the splice variant in the biological sample.

The following examples are presented in order to more fully illustrate some embodiments of the invention. They should, in no way be construed, however, as limiting the broad scope of the invention.

### EXAMPLES

### Example 1

PYY Variants

The PYY skipping 3 variant was deduced from a processed pseudogene (PPG), most likely derived from the human PYY gene by reverse transcription of PYY mRNA. The PPG found on chromosome X (chrX:49,623,718-49,624,428) (SEQ ID NO:111) is highly homologous to the known PYY gene (77.5% nucleic acid sequence homology; 82.4% amino acid sequence homology), although it lacks the sequence encoding the 3rd exon. According to this homology, we concluded that such a variant exists also in the original PYY gene.

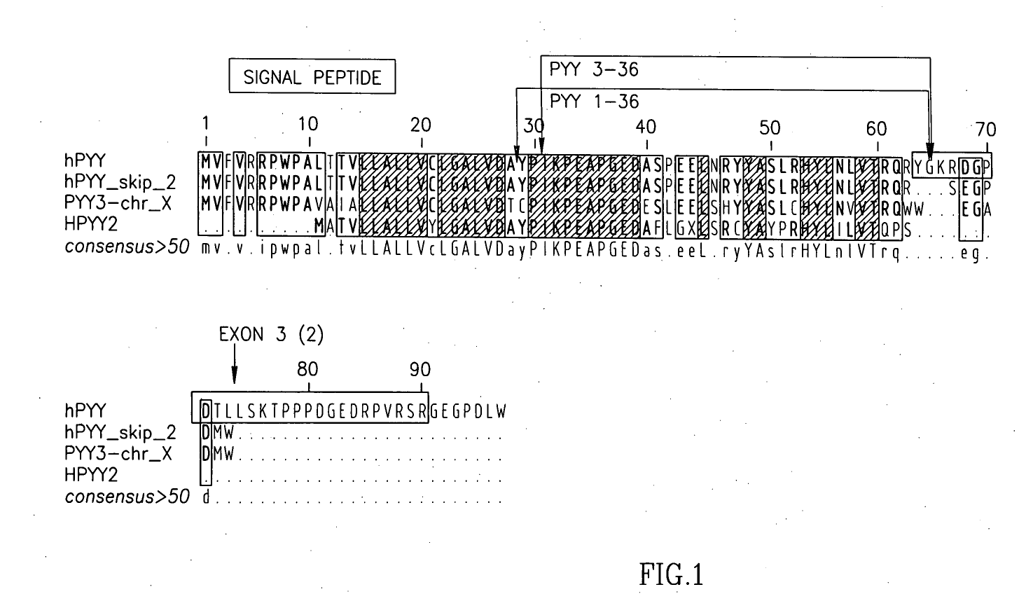

FIG. 1 shows the alignment of PYY and PYY3 known amino acid sequences to the PYY skipping exon 3 variant and HPYY corrected variant amino acid sequences of the present invention. “hPYY” is the human known PYY (SEQ ID NO:65), while “HPYY_skip_{—}2” shows the sequence of PYY skipping exon 3 (SEQ ID NO:73); “PYY3-chr_X” shows the sequence of processed PYY pseudogene (SEQ ID NO:111); and “hPYY2” shows the sequence of the PYY2 corrected variant according to the present invention (SEQ ID NO:70). The amino acid residues encoded by exon 3, skipped in the PYY skipping exon 3 variant, are shown in a rectangle. The signal peptide and the amino acid residues of PYY[1-36] and PYY[3-36] are indicated.

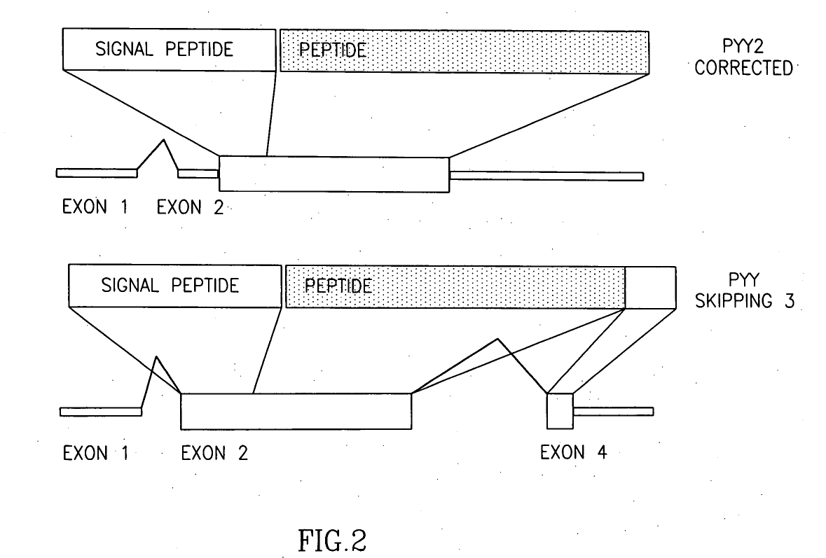

FIG. 2 shows the variant structures of the corrected PYY2 and PYY skipping exon 3, including the signal peptide. The corrected PYY2 gene is composed of 2 exons, of which the first exon is UTR. The coding regions are all within exon 2 and include the putative signal peptide and the proposed peptide (including the “corrected” Stop codon; the “correction” is actually a change of an original codon from TAG (stop codon) to GAG (Glu), which effectively extends the length of the peptide).

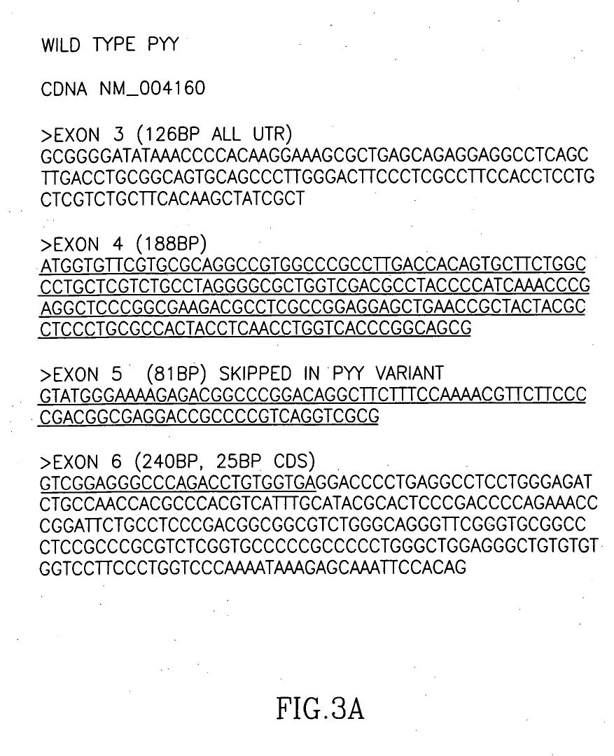

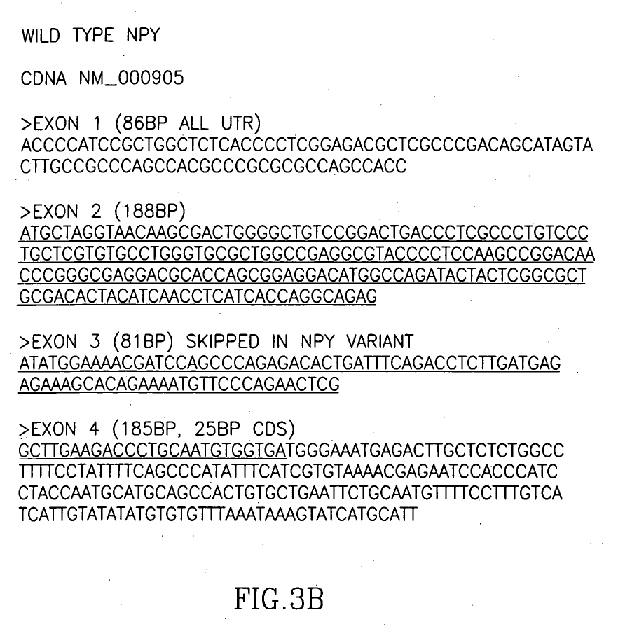

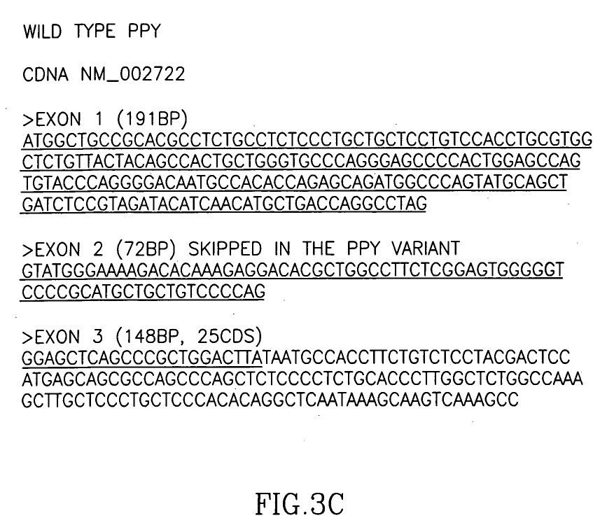

PYY skipping exon 3 is a splice variant of the original PYY gene, which consists of 4 exons, 3 of which are coding. The first exon is UTR and exons 2-4 are coding exons. According to the latest refseq reference, NM_{—}004160, SEQ ID NO:115, the PYY gene consists of 6 exons (see FIG. 3A), 3 of which are coding, and correspond to the original exons 2-4, while the two additional exons of NM_{—}004160 elongate the 5′UTR of the mRNA. The PYY skipping exon 3 variant abolishes the cleavage site between the peptide and the propeptide and adds 7 amino acids from the propeptide to the C-terminus of the peptide. The skipping exon of the PYY skipping exon 3 splice variant of the present invention is shown in FIG. 3A (marked as “exon 5”, according to a refseq accession number NM_{—}004160). In this Figure the coding sequences are underlined, as opposed to the uncoding UTRs.

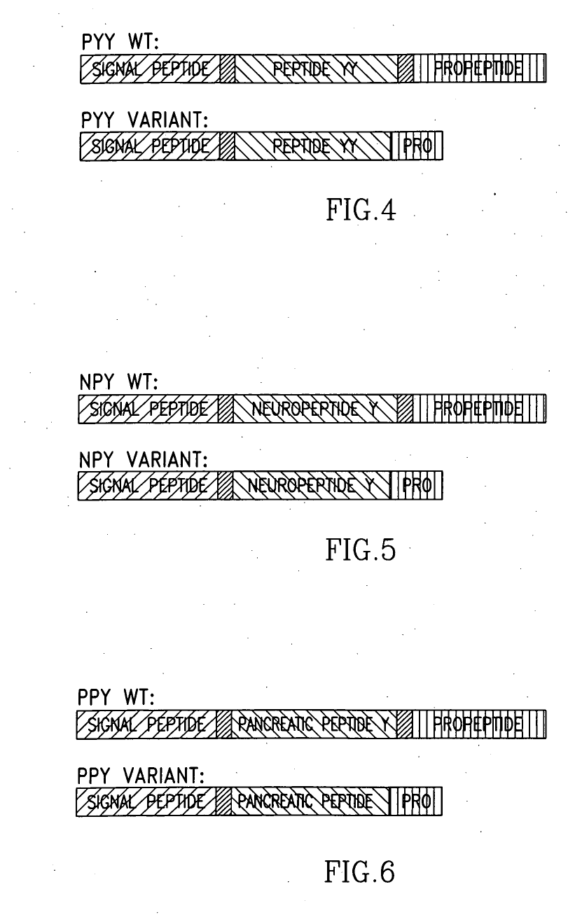

PYY skipping exon 3 splice variant of the present invention results from alternative splicing of the PYY gene thereby causing skipping over exon 3. This skipping of exon 3 (81 bp—in frame) causes a truncation of the PYY peptide's C-terminus by 1 amino acid and elimination of the cleavage site (of peptide convertase) that produces the mature peptide. The variant also eliminates most of the pro-peptide of the precursor protein. The variant encodes a product, which includes most of the functional peptide, lacks the last Tyr residue and adds the 7 C-terminal amino acid residues of the preproPYY. Schematic comparison between the known PYY and the PYY skipping exon 3 of the present invention is given in FIG. 4. In FIG. 4 the signal peptide is marked with wide upward diagonal lines; pro-peptide sequence is marked with vertical lines; the active YY peptide sequence is marked with wide downward diagonal lines; and cleavage sites are marked with light upward diagonal lines. As can be seen from FIG. 4, the C-terminal cleavage site of the known PYY is missing from the variant, and is replaced by the 7 C-terminal amino acids from the known pro-peptide PYY.

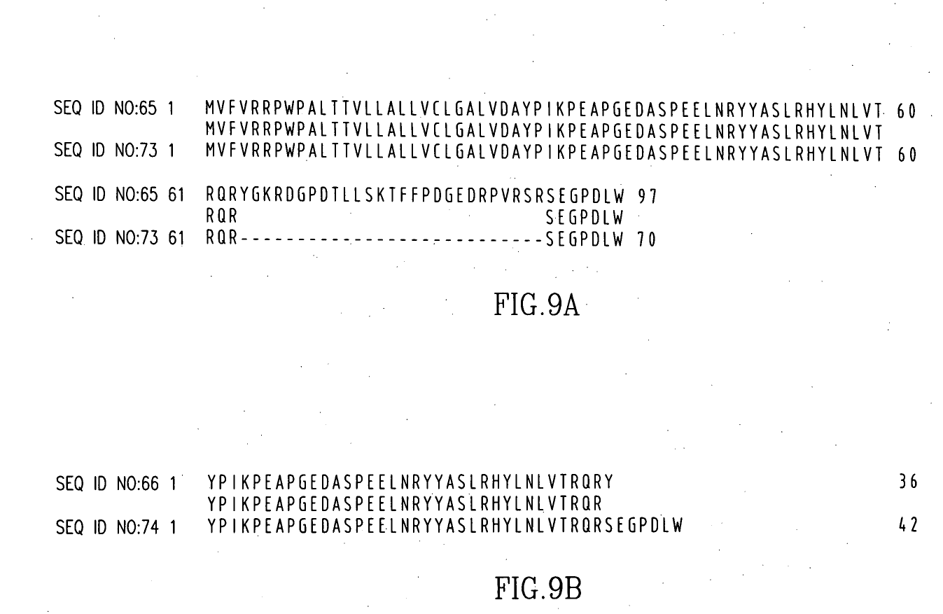

FIG. 9 shows the alignment between the known PYY protein and the PYY skipping exon 3 variant of the present invention. In FIG. 9A the comparison between the known preproPYY (SEQ ID NO:65) and the preproPYY variant skipping 3 (SEQ ID NO:73) is shown. The two proteins share the first N-terminal 63 amino acids and the last C-terminal 7 amino acids. The preproPYY variant skipping 3 has a new edge, bridging together the two parts above. In FIG. 9B the comparison between the known PYY [1-36] (SEQ ID NO:66) and the PYY variant skipping 3 [1-42] (SEQ ID NO:74) is shown. The two proteins share the first N-terminal 35 amino acids. The PYY variant skipping 3[1-42] has a new edge, bridging together the N-terminal sequence as above and the last C-terminal 7 amino acids, derived from the propeptide. The comparison between the PYY [3-36] (SEQ ID NO:67) and the PYY variant skipping 3 [3-42] (SEQ ID NO:75) is not shown, but is identical to the above, except the cleavage of the two N-terminal amino acids from both proteins.

### Example 2

NPY Variants

The SEQ ID NOS of the NPY variants of the present invention are as follows: the precursor NPY variant is depicted in SEQ ID NO:87; the mature NPY variant [1-42] is depicted in SEQ ID NO:101; and the processed active NPY variant [3-42] is depicted in SEQ ID NO:103.

NPY variant of the present invention is a splice variant of the original NPY gene, which consists of 4 exons, 3 of which are coding, as illustrated in FIG. 3B. The first exon is UTR and exons 2-4 are the coding exons. In FIG. 3B the coding sequences are colored in blue and underlined, while the uncoding UTRs are colored in red. The skipping exon 3 variant abolishes the cleavage site between the peptide and the propeptide and adds 7 amino acids from the propeptide to the C-terminus of the peptide.

NPY splice variant results from alternative splicing of the NPY gene thereby causing skipping over exon 3. This skipping of exon 3 (81 bp—in frame) results in truncating the NPY peptide's C-terminus by 1 amino acid and eliminating the cleavage site (of peptide convertase) that produces the mature peptide. The variant also eliminates most of the pro-peptide of the precursor protein. The variant encodes a product, which includes most of the functional peptide, lacking the last Tyr residue and added with the C-terminal 7 amino acid residues of the preproNPY. Schematic comparison between the known NPY and the NPY skipping exon 3 of the present invention is given in FIG. 5. In FIG. 5 the signal peptide is marked with wide upward diagonal lines; pro-peptide sequence is marked with vertical lines; the active YY peptide sequence is marked with wide downward diagonal lines; and cleavage sites are marked with light upward diagonal lines. As can be seen from FIG. 5, the C-terminal cleavage site of the known NPY is missing from the variant, and is replaced by 7 C-terminal amino acids from the known pro-peptide NPY.

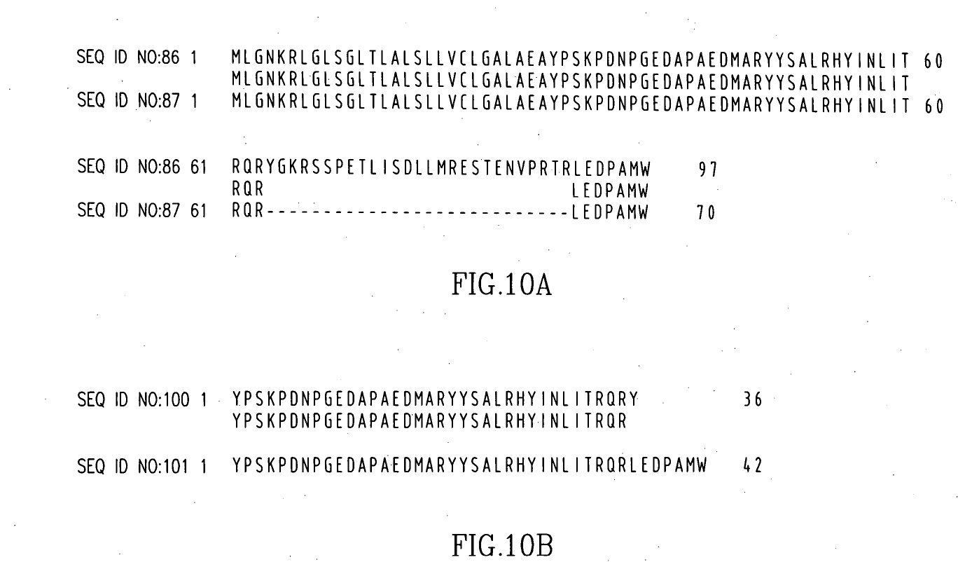

FIG. 10 shows the alignment between the known NPY protein and the NPY skipping exon 3 variant of the present invention. In FIG. 10A the comparison between the known preproNPY (SEQ ID NO:86) and the preproNPY variant skipping 3 (SEQ ID NO:87) is shown. The two proteins share the, first N-terminal 63 amino acids and the last C-terminal 7 amino acids. The preproNPY variant skipping 3 has a new edge, bridging together the two parts above. In FIG. 10B the comparison between the mature known NPY [1-36] (SEQ ID NO:100) and the mature NPY variant skipping 3 [1-42] (SEQ ID NO:101) is shown. The two proteins share the first N-terminal 35 amino acids. The NPY variant skipping 3 [1-42] has a new edge, bridging together the N-terminal sequence as above and the last C-terminal 7 amino acids, derived from the propeptide. The comparison between the processed NPY [3-36] form (SEQ ID NO:103), where the N-terminal YP amino acids are cleaved, and the similarly processed NPY variant skipping 3[3-42] (SEQ ID NO:104) is not shown, but is identical to the above, except the cleavage of the two N-terminal amino acids from both proteins.

### Example 3

PPY Variants

The SEQ ID NOS of the PPY variants of the present invention are as follows: the precursor PPY variant is depicted in SEQ ID NO:91; the mature PPY variant [1-42] is depicted in SEQ ID NO:105; and the processed active PPY variant [3-42] is depicted in SEQ ID NO:107.

PPY variant of the present invention is a splice variant of the original PPY gene, which consists of 3 coding exons, as illustrated in FIG. 3C. The 5′ UTR are not represented in the refseq accession number NM_{—}002722 (SEQ ID NO:117), as shown in FIG. 3C. In FIG. 3C the coding sequences are colored in blue and underlined, while the uncoding UTRs are colored in red. The PPY skipping exon 2 variant, similarly to skipping exon 3 in PYY and NPY variants of the present invention, as described above, abolishes the cleavage site between the peptide and the propeptide and adds 7 amino acids from the propeptide to the C-terminus of the peptide.

PPY splice variant results from alternative splicing of the PPY gene thereby causing skipping over exon 3 (exon 2 in the refseq NM_{—}002722). This skipping of exon 3 (72 bp—in frame) results in truncating the PPY peptide's C-terminus by 1 amino acid and eliminating the cleavage site (of peptide convertase) that produces the mature peptide. The variant also eliminates most of the pro-peptide of the precursor protein. The variant encodes a product, which includes most of the functional peptide, lacking the last Tyr residue and added with the 7 C-terminal amino acid residues of the preproPPY. Schematic comparison between the known PPY and the PPY skipping exon 3 of the present invention is given in FIG. 6. In FIG. 6 the signal peptide is marked with wide upward diagonal lines; pro-peptide sequence is marked with vertical lines; the active YY peptide sequence is marked with wide downward diagonal lines; and cleavage sites are marked with light upward diagonal lines. As can be seen from FIG. 6, the C-terminal cleavage site of the known PPY is missing from the variant, and is replaced by the 7 C-terminal amino acids from the known pro-peptide PPY.

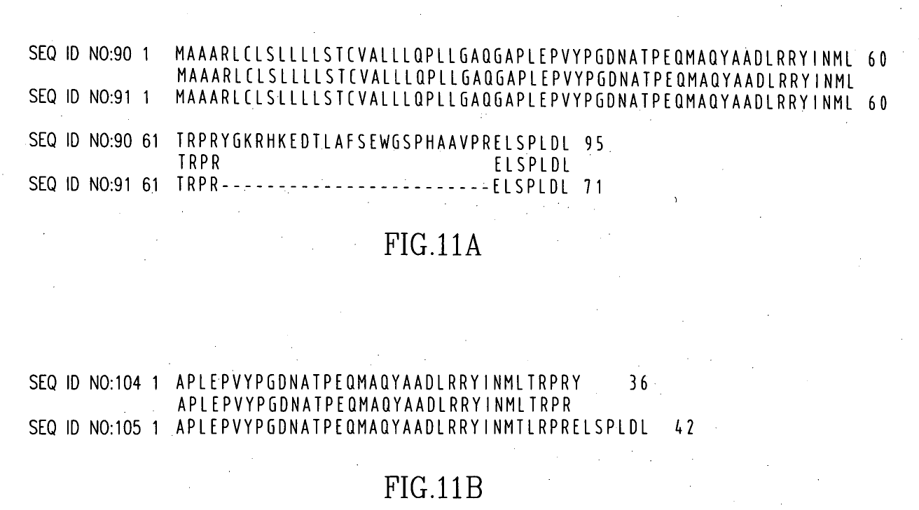

FIG. 11 shows the alignment between the known PPY protein and the PPY skipping exon 2 variant of the present invention. In FIG. 11A the comparison between the known preproPPY (SEQ ID NO:90) and the preproPPY variant skipping 2 (SEQ ID NO:91) is shown. The two proteins share the first N-terminal 64 amino acids and the last C-terminal 7 amino acids. The preproPPY variant skipping 2 has a new edge, bridging together the two parts above. In FIG. 11B the comparison between the mature known PPY [1-36] (SEQ ID NO:104) and the mature PPY variant skipping 2 [1-42] (SEQ ID NO:105) is shown. The two proteins share the first N-terminal 35 amino acids. The PPY variant skipping 2 [1-42] has a new edge, bridging together the N-terminal sequence as above and the last C-terminal 7 amino acids, derived from the propeptide. The comparison between the processed PPY [3-36] form (SEQ ID NO:106), where the N-terminal AP amino acids are cleaved, and the similarly processed PPY variant skipping 2 [3-42] (SEQ ID NO:107) is not shown, but is identical to the above, except the cleavage of the two N-terminal amino acids from both proteins.

### Example 4

Amylin Variants

The Amylin variants of the present invention are depicted in SEQ ID NOS:95 and 97, corresponding to the protein sequences of an Amylin variant 1 and 2, respectively. The corresponding nucleic acid sequences is given in SEQ ID NOS:94 and 96 for Amylin variant 1 and 2, respectively. The protein sequences of the Amylin variants 1 and 2 are shown in FIGS. 8A and 8B, respectively, where the unique amino acid sequence of the variants is marked by rectangle.

As showed by Hoppener et al., 1994, the original human Amylin gene contains 3 exons, encoding a precursor protein of 89 amino acids (SEQ ID NOS:92 and 93, respectively). This gene structure is conserved also in rat, where the 3 exons of the rat Amylin gene encode a 93 amino acids of precursor protein.

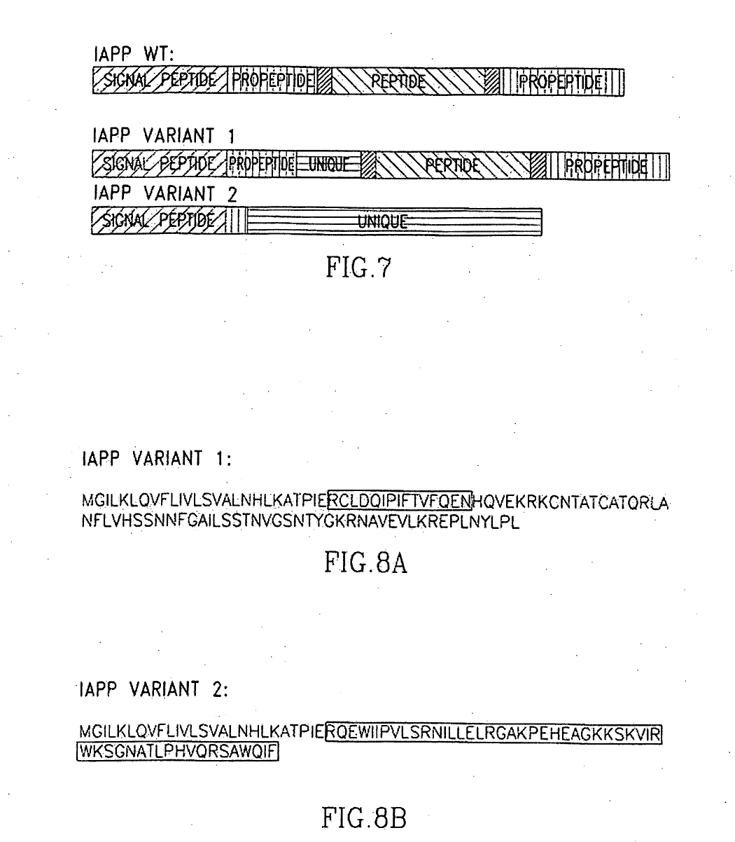

The Amylin Variant 1 (SEQ ID NO:95) is created by alternative splicing of the original human Amylin gene, causing an insertion of a novel, in-frame, 42 bp exon between exons 2 and 3 of the IAPP gene. This insertion replaces Ser 27 in the precursor protein with a unique amino acid sequence: RCLDQIPIFTVFQEN (SEQ ID NO:98). This adds 15 unique amino acids insertion to the WT pro-peptide sequence. Without being bound to theory, the precursor Amylin variant 1 of the present invention is alternatively processed to give rise to a modified mature Amylin with agonistic activities. Schematic comparison between the known Amylin and the Amylin variant 1 of the present invention is given in FIG. 7. In FIG. 7 the signal peptide is marked with wide upward diagonal lines; pro-peptide sequence is marked with vertical lines; peptide sequence is marked with wide downward diagonal lines; and cleavage sites are marked with light upward diagonal lines. The unique 15 amino acid insertion is marked with horizontal lines.

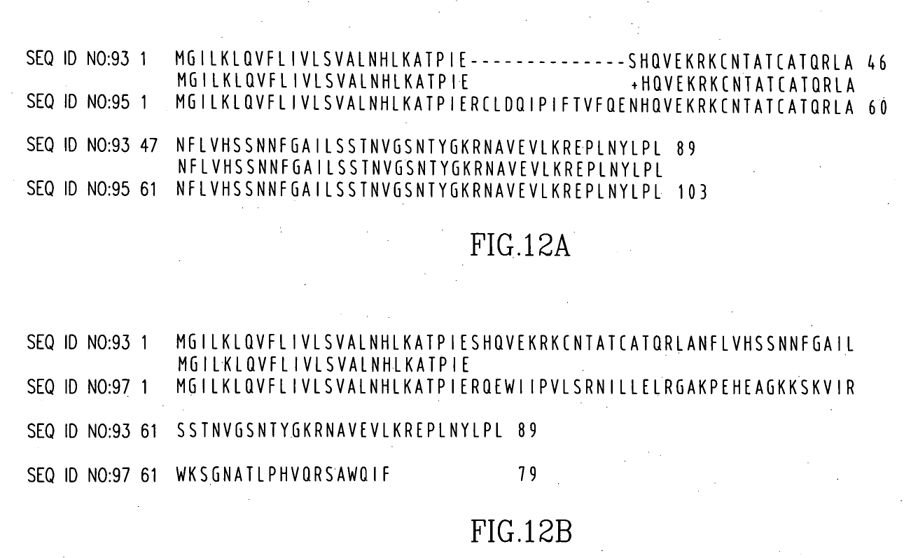

FIG. 12A shows the alignment between the known Amylin protein (SEQ ID NO:93) and the Amylin variant 1 of the present invention (SEQ ID NO:95). As can be seen from the sequence alignment, the unique insertion of 15 amino acids RCLDQIPIFTVFQEN, replaces the Ser27 of the known Amylin, giving rise to a protein of total length of 103 amino acids, as compared to a 89-amino acid long known protein.

The Amylin Variant 2 (SEQ ID NO:97) is created by alternative splicing causing an insertion of a novel 92 bp exon between exons 2 and 3 of the Amylin gene. This insertion changes the frame of the ORF and replaces the C-terminus of the Amylin protein, starting with Ser27 in the precursor protein, by a unique amino acid sequence RQEWIIPVLSRNILLELRGAKPEHEAGKKSKVIRWKSGNATLPHVQRSAWQIF (SEQ ID NO:99). This replacement eliminates all of the functional peptide but retains all of the original Signal sequence and part of the N-terminal pro-peptide. The novel exon included between the original exons 2 and 3 of the Amylin gene is a putative duplication of exon 2 and is also present and 5′ extended in Chicken Amylin (accession number L16955, SEQ ID NO:108).

Schematic comparison between the known Amylin and the Amylin variant 2 of the present invention is given in FIG. 7. In FIG. 7 the signal peptide is marked with wide upward diagonal lines; pro-peptide sequence is marked with vertical lines; peptide sequence is marked with wide downward diagonal lines; and cleavage sites are marked with light upward diagonal lines. The unique 53 amino acid insertion is marked with horizontal lines. As can be seen from FIG. 7, the unique amino acid sequence of an Amylin variant 2 replaces the original Amylin active peptide and the C-terminal propeptide, while the original signal peptide and a part of the N-terminal propeptide are remained. Without being bound to theory, the new Amylin variant 2 of the present invention can be used for diagnosis, prognosis, monitoring of the diseases and conditions related to abnormal Amylin expression, as described in other sections of this application.

FIG. 12B shows the alignment between the known Amylin protein (SEQ ID NO:93) and the Amylin variant 2 of the present invention (SEQ ID NO:97). As can be seen from the sequence alignment, the unique insertion of 53 amino acids RQEWIIPVLSRNILLELRGAKPEHEAGKKSKVIRWKSGNATLPHVQRSAW QIF, replaces the C-terminal part of the known Amylin, starting from the Ser27, giving rise to a new protein of total length of 79 amino acids.

### Example 5

mRNA Expression of PYY, PPY and NPY Variants

The mRNA expression of the PYY, NPY and PPY variants of the present invention was demonstrated by PCR, analysis using cDNA obtained by reverse transcription of total RNA from the following tissue samples:
1. Female Urogenital pool—a pool of 3 cervix RNA samples, mixed tumor and normal origin (in-house tissue samples), normal uterus, ovary and placenta derived RNA. (Biochain, www.biochain.com).
2. Male Urogenital pool—a pool of prostate, testis and kidney RNA samples (Biochain—Normal).
3. Brain with or without assorted internal organs pool—a pool of brain added with Lung, Breast, Liver derived RNA samples (Biochain—Normal).
4. Intestine and Pancreas derived RNA(Biochain—Normal,).
5. A pool of cell-line derived RNA samples from the cell-lines: DLD, MiaPaCa, HT29, THP1, MCF7 (ATCC, USA).
6. Pancreas—one sample of pancreas derived RNA (Biochain—Normal)
7. Colon—a pool of 5 colon derived RNA of mixed tumor and normal origin, added with one normal intestine derived RNA sample (in-house tissue samples).
8. Liver and spleen—one sample of liver derived RNA (Biochain—Normal), one sample of spleen derived RNA (Biochain—Normal), added with one sample of HepG2 cell line (liver tumor,—ATCC, USA) derived RNA.
   Reverse Transcription:

RNA was incubated with a random hexamer primer mix (Invitrogen), denaturated at 70° C. for 5 minutes and transferred to 4° C. for hexamer annealing. Reverse transcription was done by Superscript II Reverse transcriptase (Invitrogen), in the presence of RNAsin™ (Promega) at 37° C. for 1 hour. Reaction was terminated by enzyme deactivation on beads (Promega).

Amplification of Splicing Products

As described in Table 2A-C below, for each variant tested, specific oligonucleotide primers designed from its flanking exons were used. Amplification was performed for 35 cycles, consisting of 94° C. for 45 sec, annealing at a primer specific temperature (4° C. below the primer's TM) for 45 sec, and extension at 72° C. for 1 min. The cycle was ended by one stage of gap filling at 72° C. for 10 min's. The products were resolved on 2% agarose gel and confirmed by sequencing.

TABLE 2A

Primers used for PYY skipping exon 3 variant
(SEQ ID NO:82)

| | |
|---|---|
| Forward primer | CTATCGCTATGGTGTTCGTGCG |
| | (SEQ ID NO:118) |
| Reverse primer | CACCACAGGTCTGGGCCC |
| | (SEQ ID NO:119) |
| TM | 61 |
| Expected PCR product size of | 429, |
| known mRNA (bp) | 301 |
| Expected PCR product size of PYY | 220 |
| variant (bp) | |

TABLE 2B
Primers used for NPY (SEQ ID NO:85)

| | |
|---|---|
| Forward primer | ACCCGGGCGAGGACG |
| | (SEQ ID NO:120) |
| Reverse primer | CGTTTTACACGATGAAATATGGGC |
| | (SEQ ID NO:121) |
| TM | 60 |
| Expected PCR product size of | 255 |
| known mRNA (bp) | |
| Expected PCR product size of NPY | 174 |
| variant (bp) | |

TABLE 2C
Primers used for PPY (SEQ ID NO:89)

| | |
|---|---|
| Forward primer | GCACGCCTCTGCCTCTCC |
| | (SEQ ID NO:122) |
| Reverse primer | GCTGGCGCTGCTCATGG |
| | (SEQ ID NO:123) |
| TM | 61 |
| Expected PCR product size of | 320 |
| known mRNA (bp) | |
| Expected PCR product size of PPY | 248 |
| variant (bp) | |

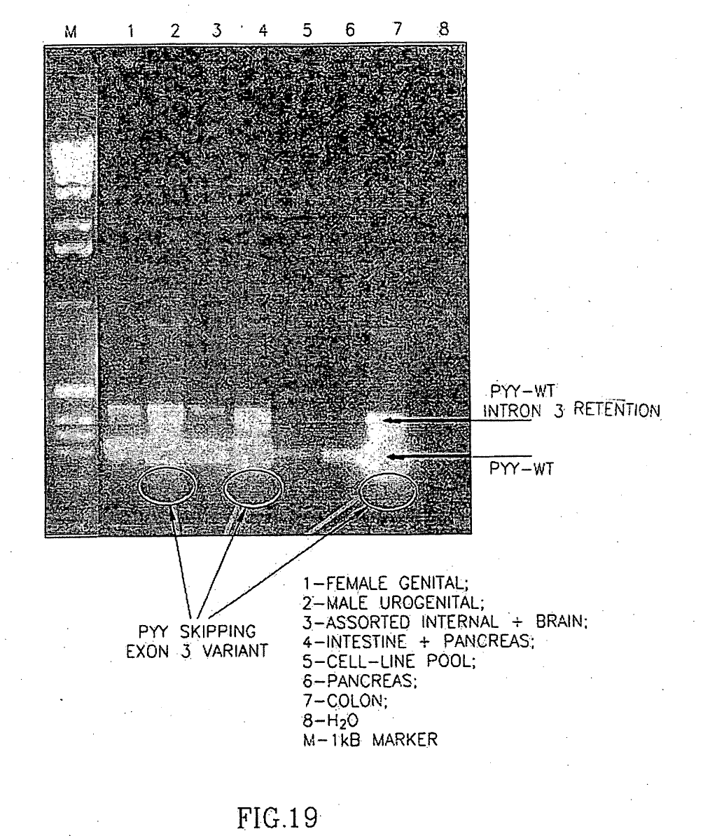

FIG. 19 shows the expression of the PYY skipping exon 3 variant mRNA using PCR analysis in tissue samples 1-7 as described above. Lane 8 is the H₂O negative control. M is a 1 KB marker. Known PYY isoforms are marked as “PYY-WT intron 3 retention” (SEQ ID NO:124), and “PYY-WT”. The PYY skipping exon 3 variant mRNA was expressed in male urogenital tissues (lane 2), intestine and pancreas (lane 4) and colon (lane 7).

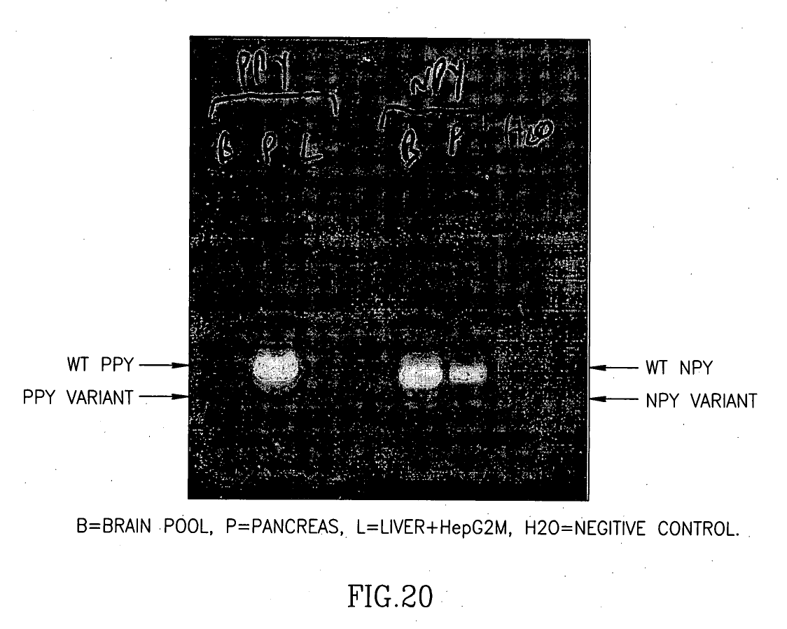

FIG. 20 shows the expression of the NPY variant and PPY variant mRNAs using PCR analysis. For the NPY variant analysis tissue samples 3 (brain only), and 6 as described above were used. For the PPY variant analysis tissue samples 3 (brain only), 6 and 8 as described above were used. The NPY variant mRNA was expressed in both tissue samples tested, brain and pancreas. The PPY variant mRNA was expressed only in pancreas.

### Example 6

Generating Splice Variant Polypeptides

Polypeptides corresponding to the amino acid sequence of the PYY splice variants and/or NPY variants and/or PPY variants and/or Amylin variants are synthesized by the solid phase method as previously described (Merrifield, R. B., Chem. Soc. 85:2149, 1965; Stewart and Young, Solid Phase Peptide Synthesis, Freeman, San Francisco, 1969, pp. 27-66). It is also possible to obtain the desired polypeptides by using recombinant DNA techniques (Sambrook et al., 1989 cited above).

### Example 7

PYY Variants In-Vitro Stability Study

The stability of sterile PYY[3-36] and PYY variant [3-42] peptide solutions was tested in Alzet pump model during 28 days of incubation at 37° C. (Alzet in-vitro protocol, DURECT Corporation, Calif., USA) following the analysis of the solutions by HPLC. In order to treat ob/ob mice with PYY variants by continuous infusion, the peptides must be soluble and the solution must be stable during the treatment period.

Solutions prepared in saline, as reported by Pittner et al., Intr. J. Obes. 2004, were chemically and physically unstable, since most (>95%) of the peptide aggregated and was insoluble after 6 days of incubation.

In the present study, much better stability was achieved when the peptides were dissolved in 20 mM acetate buffer at pH 4.0. Both the WT PYY[3-36] and the PYY variant [3-42] of the present invention were analyzed in the in-vitro stability studies using two dosages as follows: the low dose of PYY[3-36] was 3.3 mg/ml; the low dose of PYY variant [3-42] was 3.9 mg/ml; the high dose of PYY[3-36] was 10.6 mg/ml; the high dose of PYY variant [3-42] was 12.8 mg/ml. In all the cases 2 or 3 different experiments were carried out.

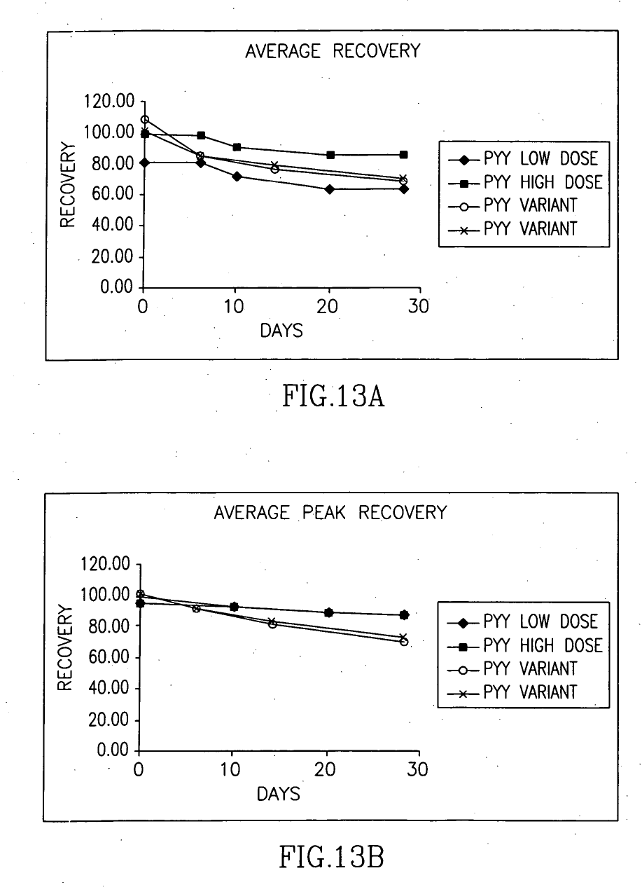

The results, demonstrating the improved recovery and purity of the peptides, are presented in Table 3 below and in FIGS. 13A-B. The calculation of the percentage of the recovery and the purity was carried out as follows: Recovery %=100× observed concentration/expected concentration as analyzed by HPLC; Purity %=100× the area under the peak of the main compound/total area under the peaks.

Table 3 shows the average percentage of the recovery and the average percentage of the purity for both, WT PYY[3-36] and the PYY variant of the invention [3-42] in aceteate buffer (20 mM pH4.0), low and high dosages, during the 28 days of the experiment.

TABLE 3
Stability study in-vitro in acetate buffer (20 mM pH 4.0)

| | Recovery (%) | Purity (%) |
|---|---|---|
| | (average ± stdev) | (average ± stdev) |
| PYY low dose | 63.6 ± 1.4 | 86.6 ± 0.02 |
| PYY high dose | 85.9 ± 1.2 | 85.8 ± 0.1 |
| PYY variant low dose | 69.2 ± 1.3 | 69.2 ± 0.6 |
| PYY variant high dose | 70.3 ± 3.1 | 72.3 ± 0.2 |

FIG. 13 shows the results of the in-vitro stability study for the WT PYY[3-36] and the PYY variant of the invention [3-42] in aceteate buffer (20 mM pH4.0). FIG. 13A shows the average recovery while FIG. 13B shows the average peak purity of the WT PYY[3-36] and the PYY variant of the invention [3-42] during the 28 days of the experiment. The color code is as follows: PYY[3-36] low dose is shown in dark blue; PYY[3-36] high dose is shown in pink; PYY variant [3-42] low dose is shown in yellow; PYY variant [3-42] high dose is shown in light blue.

As can be seen from the Table 3 and from FIG. 13, relative stability of WT PYY[3-36] and the PYY variant of the invention [3-42] were achieved, demonstrating both improved recovery ratio and improved purity of the peptides.

### Example8

Production of Polyclonal Antibodies, Specific to PYY Variants

To produce polyclonal Antibodies specific for the PYY skipping exon 3 variant of the invention, the following peptides were used to immunize rabbits: P3S (SEQ ID NO: 109) CRSEGPDLW, derived from the unique tail of PYY skipping exon 3 variant; and P3J (SEQ ID NO:110) RQRSEGGCGGRQRSEG, derived from the junction between the last three amino acids from the common sequence in the known PYY and in the PYY skipping exon 3 variant and the three first amino acids of the unique tail of PYY skipping exon 3 variant, duplicated and separated with a four amino acids of a spacer. The structure of the P3J peptide is shown in FIG. 18, where the common sequence is shown in bold; the sequence unique to the PYY variant is underlined; the remaining sequence is a spacer.

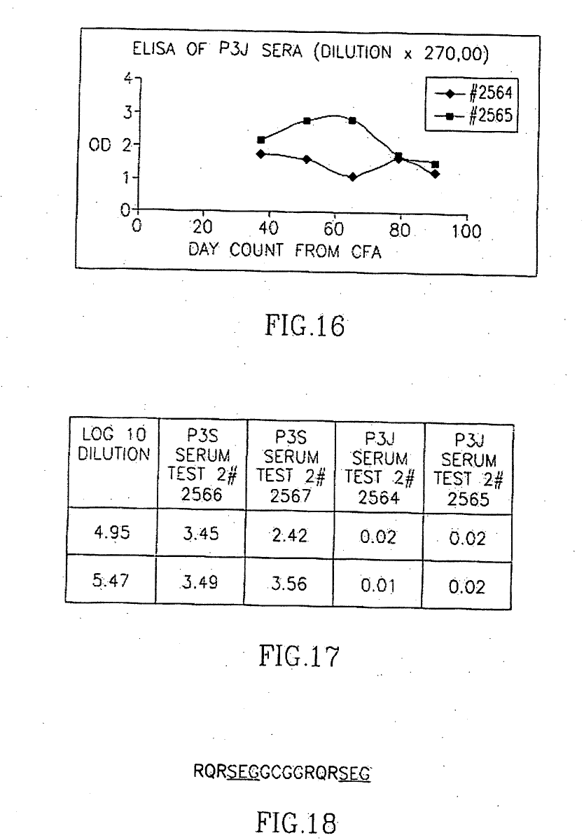

Four rabbits were immunized: two with P3S and two with P3J peptides, both of which were KLH conjugated and 95% purified peptides. The serum was collected after several booster shots. Then booster shots were given every two weeks and serum collected 7 days after each shot. The sera were then checked by ELISA, with the peptide used for immunization as well as with the PYY skipping exon 3 variant peptide. As shown in FIGS. 15-16, both sera reacted positively with their respective peptide injected. As shown in FIGS. 17, P3S serum also detected specifically the PYY skipping exon 3 variant peptide by ELISA test.

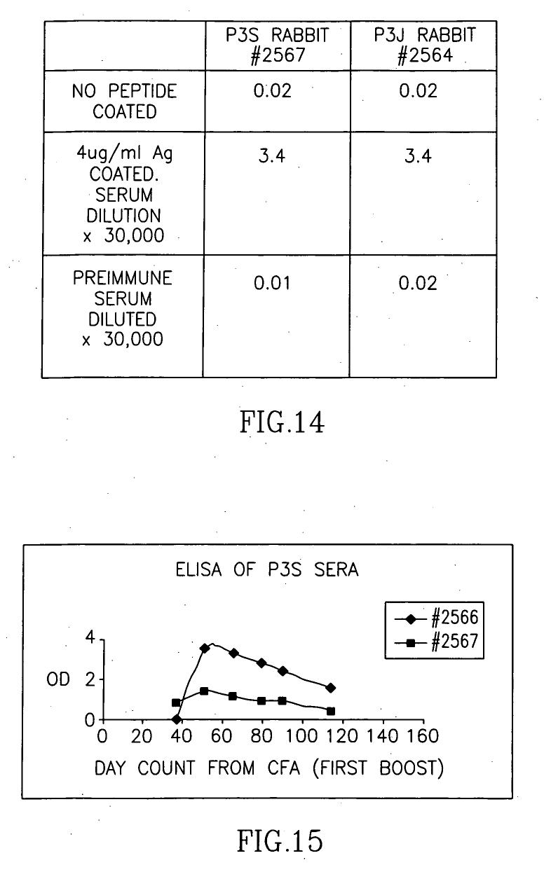

The following non-limiting method was used for the ELISA. Briefly the lyophilized peptides were weighed, dissolved into water and diluted to a final concentration of 4 ug/ml into 50 mM bicarbonate buffer pH9.6. The ELISA plates were coated with the peptides (100 μl/well in triplicates) and incubated overnight at 4° C. under shaking. The following day, coating solution was discarded and the plates washed three times with washing buffer (300 ul/well PBS-Tween 0.05%). Blocking solution (PBST containing 3% BSA) was then added into each well (200 ul/well) and the plates were incubated at 37° C. under shaking for 1 h. Meantime the sera were diluted×160 000 into 1% BSA-PBST and then added to the wells (100 ul/well) after the plates were washed as described previously. Incubation of the coated plates with the sera was performed at 37° C. under shaking for 1 h. After the incubation, the plates were washed again and a secondary antibody (Goat anti Rabbit HRP conjugated) was added (100 ul/well) diluted ×50 000 into 1% BSA-PBST. The plates were incubated at 37° C. for 1 h under shaking and washed as described previously. Finally Tetra-Methyl-Benzidine (TMB) substrate (Zymed, Cat no:2023) was added (100 ul/well) and the plates were incubated covered from light for 10 min. To stop the reaction, a solution of 4N sulfuric acid was added (100 ul/well) and the absorbance was read at 450 nm (reference 620 nm). As a negative control in all the ELISA experiments, the sera were checked on wells uncoated with peptide as well as preimmune serum on coated wells. As shown in FIG. 14, in both experiments and for both, P3S and P3J, the OD value obtained was very low, as compared to these obtained with serum of immunized rabbits checked on plates coated with 4 μg/ml with the respective peptides (FIG. 14).

FIG. 15 shows the results of ELISA test of P3S sera on the two antigens injected for both rabbits. Rabbit serum #2566 showed a better response to the respective antigen than rabbit serum #2567.

FIG. 16 shows results of ELISA test of P3J sera on the two antigens injected for both rabbits. Both rabbits showed similar OD values over the time during several boosts.

FIG. 17 shows OD results of ELISA test performed with PYY skipping exon 3 [3-42] variant peptide coated on the plate. Sera from both rabbits immunized with P3S and P3J were used. As can be seen from FIG. 17, P3S serum can detect specifically the PYY skipping exon 3 [3-42] variant peptide.

### Example 9

Assessment of Treatment Effects on Body Weight and Food Intake by PYY Variants

Assessment of potential treatment effect on body weight and food intake by PYY variants of the present invention is performed under blinded conditions. The test system includes 40 ob/ob female mice, each about 50 g & 12 weeks of age at study initiation, in 5 groups (n=8), housed 4 per cage.

The first week, representing the first stage of the experiment, is the first acclimatization stage, when the 12 hours light and dark cycles regime is applied at the same hours of the day (light cycle is between 6:30-18:30). The viability check and clinical signs observations are checked daily.

The second week of the experiment, is the second acclimatization stage, when the same 12 hours/12 hours light and dark cycles regime is applied. At this stage, every day at the same time each day, the following parameters are assessed: daily viability check and clinical signs observations, including for example, changes in skin, fur, eyes, mucous membranes, secretions, activity and response to handling, as well as any unusual behavior, sleep and coma; body weight measurement 3 times/week; and food consumption measurement 1 time/daily for 5 days a week.

The third stage, days 15-43 (weeks 3-6 of the experiment), is the test compound administration stage.

On day 15, an Alzet mini-osmotic pump (model 2004), is surgically inserted into each test animal, under general anesthesia. The pump is then used for continuous administration of test compounds on days 15-43 (total of 28 days), as described in Table 4 below.

TABLE 4

| | | | Solution |
|---|---|---|---|
| Group # | | Dose | concentration |
| (n = 8) | Compound | (μg/Kg/day) | (mg/ml)* |
| 1 | Saline | — | — |
| 2 | PYY₍₃₋₃₆₎ | 300 | 2.5 |
| 3 | PYY₍₃₋₃₆₎ | 1000 | 8.3 |
| 4 | PYY Variant₍₃₋₄₂₎ | 300 | 2.9 |
| 5 | PYY Variant₍₃₋₄₂₎ | 1000 | 9.6 |

*The outlined concentration was calculated based on a body weight of 50 g, pump volume of 200 μl, and output of 6 μl/day. The exact concentration is adjusted prior to administration based on the actual body weight of each animal at that time.

During the whole experiment the 12 hours light and dark cycles regime is applied at the same hours of the day (light cycle is between 6:30-18:30). Every day at the same time each day, the following parameters are assessed: daily viability check and clinical signs observations, including for example, changes in skin, fur, eyes, mucous membranes, secretions, activity and response to handling, as well as any unusual behavior, sleep and coma; body weight measurement 3 times/week; and food consumption measurement 1 time/daily for 5 days a week

At the last day of the experiment, day 43, the mice are sacrificed after night fast and undergo gross pathological examination. The blood and organs are collected, and stored at −80° C.

Without wishing to be limited by a single hypothesis, the mice receiving PYY variant of the present invention gain significantly less weight than the control group, during 28 days of the experiment. The mice receiving PYY variant of the present invention also show significant effect of reduction in the food intake, especially during the first days of the study.

### Example 10

Insulinotropic Splice Variant Polypeptides

The splice variants of the present invention are tested in several biological systems, including conscious dog, anesthetized dog with chronic indwelling left atrial catheters, and beta TC-3 insulinoma cell line (described in D'Ambra et al., Endocrinology 126:2815-2822, 1990) in cell culture. Following a bolus injection of polypeptide in a conscious dog, the insulin secretory response above basal level is determined.

### Example 11

Glucose Dependent Splice Variant Insulin Secretagogue Activity

Dogs with glucose concentrations clamped at graded levels are assessed for their glucose-dependent insulinotropic response to the PYY splice variants and/or NPY variants and/or PPY variants and/or Amylin variants.

Varying dosages of the peptides are administered, and dosages which do not stimulate insulin release at fasting glucose concentrations of 50-75 mg/dL (such as 0.1 nmol peptides, given as a bolus) yet are able to produce a peak insulin response of one-fold above basal when given to dogs in a clamped, hyperglycemic state are determined.

The peptides may also be compared in order to determine which provides a greater insulin secretory response.

### Example 12

Splice Variants Direct Activity on Pancreatic Beta Cells

Beta TC-3 cells are cultured in serum-containing media in 48-well culture dishes to confluency. Cells are tested in Earle's balanced salt solution containing IBMX, BSA and 16.7 mM glucose with graded concentrations of the PYY splice variants and/or NPY variants and/or PPY variants and/or Amylin variants for 1 hour at 37° C. prior to supernatant collection and assay for insulin concentration.

### Example 13

Effect of PYY Variants and/or NPY Variants and/or PPY Variants and/or Amylin Variants and/or OXM Variants on Reduction of Body Weight in Animal Models

Cumulative food intake in grams is measured over the course of 24 hours following intravenous or intraperitoneai injection of a PYY variants and/or NPY variants and/or PPY variants and/or Amylin variants in fasted rats and/or sated rats. Dose-dependent decreases in food intake are determined in treated rats versus placebo treated controls, as well as single versus multiple injections. The effect of the time of day of feeding and/or administration of the splice variants is determined as well. At the conclusion of the experiment, in addition to the measurement of food intake, as well as overall body weight, gastric emptying is determined in the rats, as well, with the contents in dry weight expressed as a percentage of food intake during the feeding period. Decreases in fasting-induced refeeding following injection of PYY variants and/or NPY variants and/or PPY variants and/or Amylin variants are measured, as well.

Adult rats can also be cannulated and infused with the splice variants and placebo controls, with delivery following a 24-hour fast, or measured in non-fasted animals, and food intake is measured at multiple time points following delivery of the splice variants.

For example, Intracerebroventricular (ICV) and intranuclear administration of OXM variants to adult rats to reduce body weight and adiposity can be performed using a protocol described in Dakin C. L., et al., Am J Physiol Endocrinol Metab 283: E1173-E1177, 2002, and in Dakin C. L., et al., Endocrinology 142: 4244-4250, 2001). In these studies, twice-daily ICV administration of known OXM, 1 nmol for 7 days showed significantly less weight gain than control animals. OXM treatment caused a reduction in epididymal white adipose tissue and interscapular brown adipose tissue, and increased core temperature suggestive of enhanced energy expenditure.

### Example 14

PYY Variants and/or PPY Variants and/or NPY Variants and/or Amylin and/or GLP-1 Variants and or OXM Variants for Treatment of Metabolic Disorders

The compositions of the present invention can be delivered to the subject in need to treat metabolic disorder by any delivery route known in the art, particularly in oral, intranasal, sublingual, intraduodenal, subcutaneous, buccal, intracolonic, rectal, vaginal, mucosal, pulmonary, transdermal, intradermal, parenteral, intraperitoneal, intravenous, intramuscular and ocular systems, as well as traversing the blood-brain barrier. An example of PYY and PYY agonist delivery systems is described in US 20050009748, fully incorporated herein by reference.

The PYY variants and/or PPY variants and/or NPY variants and/or Amylin variants and/or GLP-1 variant and or OXM variants can be administered to the subject in need to treat metabolic disorders. The example of suitable methods is described in US20020141985, fully incorporated herein by reference. The above patent application discloses methods for using compositions including PYY and PYY agonist to reduce food intake, to reduce gastric emptying, to inhibit gastric acid secretion, to prevent gallbladder emptying, to inhibit pancreatic secretion, to decrease body weight, to decrease caloric efficiency, and to improve glycemic control in obese diabetic (ZDF) rats.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is to be understood that the phraseology or terminology employed herein is for the purpose of description and not of limitation. The means, materials, and steps for carrying out various disclosed chemical structures and functions may take a variety of alternative forms without departing from the invention.

It is appreciated that certain features of the invention, which are, for clarity, is described in the context of separate embodiments, may also be provided in combination in a single embodiment. Conversely, various features of the invention, which are, for brevity, described in the context of a single embodiment, may also be provided separately or in any suitable sub combination.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An isolated PYY variant having an amino acid sequence as set forth in any one of SEQ ID NOS:70-75.

2. An isolated chimeric polypeptide, comprising a first amino acid sequence being at least about 90% homologous to amino acids 1-63 of known preproPYY (SEQ ID NO:65), and a second amino acid sequence being at least about 85% homologous to amino acids 91-97 of known preproPYY (SEQ ID NO:65), wherein said first and said second amino acid sequences are contiguous and in sequential order.

3. The polypeptide of claim 2, wherein said first amino acid sequence is at least about 95% homologous to amino acids 1-63 of known preproPYY (SEQ ID NO:65).

4. An isolated chimeric polypeptide, comprising a first amino acid sequence being at least about 90% homologous to amino acids 1-35 of known PYY (SEQ ID NO:66), and a second amino acid sequence being at least 70% homologous to a peptide having the sequence SEGPDLW (SEQ ID NO:112) corresponding to amino acids 3642 of PYY skipping exon 3 [1-42] variant (SEQ ID NO:74), wherein said first and said second amino acid sequences are contiguous and in sequential order.

5. The polypeptide of claim 4, wherein said first amino acid sequence is at least about 95% homologous to amino acids 1-35 of known PYY (SEQ ID NO:66).

6. The polypeptide of claim 4, wherein said second amino acid sequence is at least about 80%-homologous to said peptide having the sequence SEGPDLW (SEQ ID NO:112).

7. The polypeptide of claim 6, wherein said second amino acid sequence is at least about 85% homologous to said peptide having the sequence SEGPDLW (SEQ ID NO:112).

8. A chimeric polypeptide comprising an edge portion of PYY skipping exon 3 variant (SEQ ID NO:74), having a length “n”, wherein n is at least about 10 amino acids, comprising the dipeptide RS, the edge portion having a sequence starting from amino acid number 35−x (numbering according to SEQ ID NO:74) and extending to amino acid number 36+((n−2)−x), in which x is an integer from 0 to n−2, wherein the value ((n−2)−x) is not larger than 6.

9. The polypeptide of claim 8, wherein n is at least about 20 amino acids in length.

10. The polypeptide of claim 9, wherein n is at least about 30 amino acids in length.

11. The polypeptide of claim 10, wherein n is at least about 40 amino acids in length.

12. A pharmaceutical composition comprising as an active ingredient a PYY Splice Variant polypeptide having an amino acid sequence as set forth in any one of SEQ ID NOS:70-75.

13. An isolated nucleic acid molecule encoding a PYY variant having a nucleic acid sequence as set forth in any one of SEQ ID NOS:80-83 or homologs thereof.

14. An expression vector comprising the polynucleotide sequence according to claim 13.

15. A host cell comprising the vector according to claim 14.

16. A pharmaceutical composition comprising as an active ingredient a nucleic acid molecule encoding a PYY variant having a nucleic acid sequence as set forth in any one of SEQ ID NOS:80-83 or homologs thereof.


SEQUENCE LISTING (124 sequences)

SEQ ID NO: 1 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 2 - DNA, 543 bp, Homo sapiens
atgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggaaataa

SEQ ID NO: 3 - protein, 114 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDFPRRGRHC

SEQ ID NO: 4 - DNA, 469 bp, Homo sapiens
atgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgattttcccagaagaggtcgccattgttgaagaacttggccgcagacatgctgatggttctttctctgatgagatgaacaccattcttgataatcttgccgccagggactttataaactggttgattcagaccaaaatcactgacaggaaataa

SEQ ID NO: 5 - protein, 37 aa, Homo sapiens
HDEFERHAEGTFTSDVSSYLEGQAAKEFIAWLVKGRG

SEQ ID NO: 6 - DNA, 111 bp, Homo sapiens
cacgatgaatttgagagacatgctgaagggacctttaccagtgatgtaagttcttatttggaaggccaagctgccaaggaattcattgcttggctggtgaaaggccgagga

SEQ ID NO: 7 - protein, 23 aa, Homo sapiens
HDEFERHAEGTFTSDFPRRGRHC

SEQ ID NO: 8 - protein, 22 aa, Homo sapiens
HDEFERHAEGTFTSDFPRRGRH

SEQ ID NO: 9 - protein, 21 aa, Homo sapiens
HDEFERHAEGTFTSDFPRRGR

SEQ ID NO: 10 - protein, 19 aa, Homo sapiens
HDEFERHAEGTFTSDFPRR

SEQ ID NO: 11 - protein, 17 aa, Homo sapiens
HAEGTFTSDFPRRGRHC

SEQ ID NO: 12 - protein, 16 aa, Homo sapiens
HAEGTFTSDFPRRGRH

SEQ ID NO: 13 - protein, 15 aa, Homo sapiens
HAEGTFTSDFPRRGR

SEQ ID NO: 14 - protein, 13 aa, Homo sapiens
HAEGTFTSDFPRR

SEQ ID NO: 15 - protein, 24 aa, Artificial
  synthetic peptide
HDEFERHAEGTFTSDFPRRGRHCX

SEQ ID NO: 16 - protein, 23 aa, Artificial
  synthetic peptide
HDEFERHAEGTFTSDFPRRGRHX

SEQ ID NO: 17 - protein, 22 aa, Artificial
  synthetic peptide
HDEFERHAEGTFTSDFPRRGRX

SEQ ID NO: 18 - protein, 20 aa, Artificial
  synthetic peptide
HDEFERHAEGTFTSDFPRRX

SEQ ID NO: 19 - protein, 18 aa, Artificial
  synthetic peptide
HAEGTFTSDFPRRGRHCX

SEQ ID NO: 20 - protein, 17 aa, Artificial
  synthetic peptide
HAEGTFTSDFPRRGRHX

SEQ ID NO: 21 - protein, 16 aa, Artificial
  synthetic peptide
HAEGTFTSDFPRRGRX

SEQ ID NO: 22 - protein, 14 aa, Artificial
  synthetic peptide
HAEGTFTSDFPRRX

SEQ ID NO: 23 - DNA, 69 bp, Homo sapiens
cacgatgaatttgagagacatgctgaagggacctttaccagtgattttcccagaagaggtcgccattgt

SEQ ID NO: 24 - DNA, 66 bp, Homo sapiens
cacgatgaatttgagagacatgctgaagggacctttaccagtgattttcccagaagaggtcgccat

SEQ ID NO: 25 - DNA, 63 bp, Homo sapiens
cacgatgaatttgagagacatgctgaagggacctttaccagtgattttcccagaagaggtcgc

SEQ ID NO: 26 - DNA, 57 bp, Homo sapiens
cacgatgaatttgagagacatgctgaagggacctttaccagtgattttcccagaaga

SEQ ID NO: 27 - DNA, 51 bp, Homo sapiens
catgctgaagggacctttaccagtgattttcccagaagaggtcgccattgt

SEQ ID NO: 28 - DNA, 48 bp, Homo sapiens
catgctgaagggacctttaccagtgattttcccagaagaggtcgccat

SEQ ID NO: 29 - DNA, 45 bp, Homo sapiens
catgctgaagggacctttaccagtgattttcccagaagaggtcgc

SEQ ID NO: 30 - DNA, 39 bp, Homo sapiens
catgctgaagggacctttaccagtgattttcccagaaga

SEQ ID NO: 31 - DNA, 24 bp, Artificial
  synthetic oligonucleotide
tttcccagaagaggtcgccattgt

SEQ ID NO: 32 - DNA, 21 bp, Artificial
  synthetic oligonucleotide
tttcccagaagaggtcgccat

SEQ ID NO: 33 - DNA, 18 bp, Artificial
  synthetic oligonucleotide
tttcccagaagaggtcgc

SEQ ID NO: 34 - DNA, 12 bp, Artificial
  synthetic oligonucleotide
tttcccagaaga

SEQ ID NO: 35 - protein, 8 aa, Artificial
  synthetic peptide
FPRRGRHC

SEQ ID NO: 36 - protein, 7 aa, Artificial
  synthetic peptide
FPRRGRH

SEQ ID NO: 37 - protein, 6 aa, Artificial
  synthetic peptide
FPRRGR

SEQ ID NO: 38 - protein, 4 aa, Artificial
  synthetic peptide
FPRR

SEQ ID NO: 39 - protein, 37 aa, Homo sapiens
HSQGTFTSDYSKYLDSRRAQDFVQWLMNTKRNRNNIA

SEQ ID NO: 40 - DNA, 111 bp, Homo sapiens
cattcacagggcacattcaccagtgactacagcaagtatctggactccaggcgtgcccaagattttgtgcagtggttgatgaataccaagaggaacaggaataacattgcc

SEQ ID NO: 41 - protein, 62 aa, Homo sapiens
HSQGTFTSDYSKYLDSRRAQDFVQWLMNTKRNRNNIAKRHDEFERHAEGTFTSDFPRRGRHC

SEQ ID NO: 42 - protein, 61 aa, Homo sapiens
HSQGTFTSDYSKYLDSRRAQDFVQWLMNTKRNRNNIAKRHDEFERHAEGTFTSDFPRRGRH

SEQ ID NO: 43 - protein, 60 aa, Homo sapiens
HSQGTFTSDYSKYLDSRRAQDFVQWLMNTKRNRNNIAKRHDEFERHAEGTFTSDFPRRGR

SEQ ID NO: 44 - protein, 58 aa, Homo sapiens
HSQGTFTSDYSKYLDSRRAQDFVQWLMNTKRNRNNIAKRHDEFERHAEGTFTSDFPRR

SEQ ID NO: 45 - DNA, 186 bp, Homo sapiens
cattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgattttcccagaagaggtcgccattgt

SEQ ID NO: 46 - DNA, 183 bp, Homo sapiens
cattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgattttcccagaagaggtcgccat

SEQ ID NO: 47 - DNA, 180 bp, Homo sapiens
cattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgattttcccagaagaggtcgc

SEQ ID NO: 48 - DNA, 174 bp, Homo sapiens
cattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgattttcccagaaga

SEQ ID NO: 49 - protein, 25 aa, Artificial
  synthetic peptide
KRHDEFERHAEGTFTSDFPRRGRHC

SEQ ID NO: 50 - protein, 24 aa, Artificial
  synthetic peptide
KRHDEFERHAEGTFTSDFPRRGRH

SEQ ID NO: 51 - protein, 23 aa, Artificial
  synthetic peptide
KRHDEFERHAEGTFTSDFPRRGR

SEQ ID NO: 52 - protein, 21 aa, Artificial
  synthetic peptide
KRHDEFERHAEGTFTSDFPRR

SEQ ID NO: 53 - DNA, 75 bp, Artificial
  synthetic oligonucleotide
aaacgtcacgatgaatttgagagacatgctgaagggacctttaccagtgattttcccagaagaggtcgccattgt

SEQ ID NO: 54 - DNA, 72 bp, Artificial
  synthetic oligonucleotide
aaacgtcacgatgaatttgagagacatgctgaagggacctttaccagtgattttcccagaagaggtcgccat

SEQ ID NO: 55 - DNA, 69 bp, Artificial
  synthetic oligonucleotide
aaacgtcacgatgaatttgagagacatgctgaagggacctttaccagtgattttcccagaagaggtcgc

SEQ ID NO: 56 - DNA, 63 bp, Artificial
  synthetic oligonucleotide
aaacgtcacgatgaatttgagagacatgctgaagggacctttaccagtgattttcccagaaga

SEQ ID NO: 57 - DNA, 1128 bp, Homo sapiens
acagagcttaggacacagagcacatcaaaagttcccaaagagggcttgctctctcttcacctgctctgttctacagcacactaccagaagacagcagaaatgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggaaataactatatcactattcaagatcatcttcacaacatcacctgctagccacgtgggatgtttgaaatgttaagtcctgtaaatttaagaggtgtattctgaggccacattgctttgcatgccaataaataaattttcttttagtgttgtgtagccaaaaattacaaatggaataaagttttatcaaaatattgctaaaatatcagctttaaaatatgaaagtgctagattctgttattttcttcttattttggatgaagtaccccaacctgtttacatttagcgataaaattatttttctatgatataatttgtaaatgtaaattattccgatctgacatatctgcattataataataggagaatagaagaactggtagccacagtggtgaaattggaaagagaactttcttcctgaaacctttgtcttaaaaatactcagctttcaatgtatcaaagatacaattaaataaaattttcaagcttcttta

SEQ ID NO: 58 - protein, 17 aa, Homo sapiens
HDEFERHAEGTFTSDFP

SEQ ID NO: 59 - protein, 11 aa, Homo sapiens
HAEGTFTSDFP

SEQ ID NO: 60 - protein, 18 aa, Artificial
  synthetic peptide
HDEFERHAEGTFTSDFPX

SEQ ID NO: 61 - protein, 12 aa, Artificial
  synthetic peptide
HAEGTFTSDFPX

SEQ ID NO: 62 - DNA, 51 bp, Homo sapiens
cacgatgaatttgagagacatgctgaagggacctttaccagtgattttccc

SEQ ID NO: 63 - DNA, 33 bp, Homo sapiens
catgctgaagggacctttaccagtgattttccc

SEQ ID NO: 64 - protein, 17 aa, Artificial
  modified GLP-1 splice variant
HGEGTFTSDFPRRGRHC

SEQ ID NO: 65 - protein, 97 aa, Homo sapiens
MVFVRRPWPALTTVLLALLVCLGALVDAYPIKPEAPGEDASPEELNRYYASLRHYLNLVTRQRYGKRDGPDTLLSKTFFPDGEDRPVRSRSEGPDLW

SEQ ID NO: 66 - protein, 36 aa, Homo sapiens
YPIKPEAPGEDASPEELNRYYASLRHYLNLVTRQRY

SEQ ID NO: 67 - protein, 34 aa, Homo sapiens
IKPEAPGEDASPEELNRYYASLRHYLNLVTRQRY

SEQ ID NO: 68 - protein, 33 aa, Homo sapiens
MATVLLALLVYLGALVDAYPIKPEAPGEDAFLG

SEQ ID NO: 69 - protein, 15 aa, Homo sapiens
YPIKPEAPGEDAFLG

SEQ ID NO: 70 - protein, 53 aa, Homo sapiens
MATVLLALLVYLGALVDAYPIKPEAPGEDAFLGELSRCYAYPRHYLILVTQPS

SEQ ID NO: 71 - protein, 35 aa, Homo sapiens
YPIKPEAPGEDAFLGELSRCYAYPRHYLILVTQPS

SEQ ID NO: 72 - protein, 33 aa, Homo sapiens
IKPEAPGEDAFLGELSRCYAYPRHYLILVTQPS

SEQ ID NO: 73 - protein, 70 aa, Homo sapiens
MVFVRRPWPALTTVLLALLVCLGALVDAYPIKPEAPGEDASPEELNRYYASLRHYLNLVTRQRSEGPDLW

SEQ ID NO: 74 - protein, 42 aa, Homo sapiens
YPIKPEAPGEDASPEELNRYYASLRHYLNLVTRQRSEGPDLW

SEQ ID NO: 75 - protein, 40 aa, Homo sapiens
IKPEAPGEDASPEELNRYYASLRHYLNLVTRQRSEGPDLW

SEQ ID NO: 76 - DNA, 582 bp, Homo sapiens
cagcttgacctgcggcagtgcagcccttgggacttccctcgccttccacctcctgctcgtctgcttcacaagctatcgctatggtgttcgtgcgcaggccgtggcccgccttgaccacagtgcttctggccctgctcgtctgcctaggggcgctggtcgacgcctaccccatcaaacccgaggctcccggcgaagacgcctcgccggaggagctgaaccgctactacgcctccctgcgccactacctcaacctggtcacccggcagcggtatgggaaaagagacggcccggacaggcttctttccaaaacgttcttccccgacggcgaggaccgccccgtcaggtcgcggtcggagggcccagacctgtggtgaggacccctgaggcctcctgggagatctgccaaccacgcccacgtcatttgcatacgcactcccgaccccagaaacccggattctgcctcccgacggcggcgtctgggcagggttcgggtgcggccctccgcccgcgtctcggtgcccccgccccctgggctggagggctgtgtgtggtccttccctggtcccaaaataaagagcaaat

SEQ ID NO: 77 - DNA, 294 bp, Homo sapiens
atggtgttcgtgcgcaggccgtggcccgccttgaccacagtgcttctggccctgctcgtctgcctaggggcgctggtcgacgcctaccccatcaaacccgaggctcccggcgaagacgcctcgccggaggagctgaaccgctactacgcctccctgcgccactacctcaacctggtcacccggcagcggtatgggaaaagagacggcccggacaggcttctttccaaaacgttcttccccgacggcgaggaccgccccgtcaggtcgcggtcggagggcccagacctgtggtga

SEQ ID NO: 78 - DNA, 840 bp, Homo sapiens
ccagtgcgcccttgaaactttcctcgccttccacctcctgctcatctgcttcacaagctgtcgctgtggtgtcggttcgcaggtcgtggcctctcatggccacggtgcttttggccctgctcgtctacctgggggcgctggtcgacgcctaccccatcaaacccgaggctcccggcgaagacgccttcctggggtagctgagccgctgctacgcctatcctcgccactacctcatcctggtcactcagccgtcgtgagcgcaggcgcggggcgggcggacggggacccctggggctctccccctacagccccgctccaccgggggcgtggctagatctgaccgcgcccggccaggccccgccctcaggtatgggaaactaggccgcccagtcgcgcgcctctccaaaacgttcttcccagactgcgaggaccgcctcggcaggtggcggtaaaagcgcccccatcaagtcacataacatcctgcctccgagagcgcggtctggccccaccctggtccatcatcacttacgacgtctcccaggcttgcctccccggatcggattcctttcccttcgatcccgcaggccggagggcgcagacctgtggtgaggacacccgaggcctcctgggagacctgcagaccacgcccacctcatttacatgttcactcccgaccctggaaacccggatttcgcctccggacagcggcgtctgggcagggttcgggtactgcagtcccgcgtctggatgccccgcgccccctgagctgcagggctgtgtgtggtccttccctggtcccaaaataaagagcggattgcacagaaacggaaaaaaaaaaaa

SEQ ID NO: 79 - DNA, 102 bp, Homo sapiens
atggccacggtgcttttggccctgctcgtctacctgggggcgctggtcgacgcctaccccatcaaacccgaggctcccggcgaagacgccttcctggggtag

SEQ ID NO: 80 - DNA, 840 bp, Homo sapiens
ccagtgcgcccttgaaactttcctcgccttccacctcctgctcatctgcttcacaagctgtcgctgtggtgtcggttcgcaggtcgtggcctctcatggccacggtgcttttggccctgctcgtctacctgggggcgctggtcgacgcctaccccatcaaacccgaggctcccggcgaagacgccttcctgggggagctgagccgctgctacgcctatcctcgccactacctcatcctggtcactcagccgtcgtgagcgcaggcgcggggcgggcggacggggacccctggggctctccccctacagccccgctccaccgggggcgtggctagatctgaccgcgcccggccaggccccgccctcaggtatgggaaactaggccgcccagtcgcgcgcctctccaaaacgttcttcccagactgcgaggaccgcctcggcaggtggcggtaaaagcgcccccatcaagtcacataacatcctgcctccgagagcgcggtctggccccaccctggtccatcatcacttacgacgtctcccaggcttgcctccccggatcggattcctttcccttcgatcccgcaggccggagggcgcagacctgtggtgaggacacccgaggcctcctgggagacctgcagaccacgcccacctcatttacatgttcactcccgaccctggaaacccggatttcgcctccggacagcggcgtctgggcagggttcgggtactgcagtcccgcgtctggatgccccgcgccccctgagctgcagggctgtgtgtggtccttccctggtcccaaaataaagagcggattgcacagaaacggaaaaaaaaaaaa

SEQ ID NO: 81 - DNA, 162 bp, Homo sapiens
atggccacggtgcttttggccctgctcgtctacctgggggcgctggtcgacgcctaccccatcaaacccgaggctcccggcgaagacgccttcctgggggagctgagccgctgctacgcctatcctcgccactacctcatcctggtcactcagccgtcgtga

SEQ ID NO: 82 - DNA, 501 bp, Homo sapiens
cagcttgacctgcggcagtgcagcccttgggacttccctcgccttccacctcctgctcgtctgcttcacaagctatcgctatggtgttcgtgcgcaggccgtggcccgccttgaccacagtgcttctggccctgctcgtctgcctaggggcgctggtcgacgcctaccccatcaaacccgaggctcccggcgaagacgcctcgccggaggagctgaaccgctactacgcctccctgcgccactacctcaacctggtcacccggcagcggtcggagggcccagacctgtggtgaggacccctgaggcctcctgggagatctgccaaccacgcccacgtcatttgcatacgcactcccgaccccagaaacccggattctgcctcccgacggcggcgtctgggcagggttcgggtgcggccctccgcccgcgtctcggtgcccccgccccctgggctggagggctgtgtgtggtccttccctggtcccaaaataaagagcaaat

SEQ ID NO: 83 - DNA, 213 bp, Homo sapiens
atggtgttcgtgcgcaggccgtggcccgccttgaccacagtgcttctggccctgctcgtctgcctaggggcgctggtcgacgcctaccccatcaaacccgaggctcccggcgaagacgcctcgccggaggagctgaaccgctactacgcctccctgcgccactacctcaacctggtcacccggcagcggtcggagggcccagacctgtggtga

SEQ ID NO: 84 - DNA, 550 bp, Homo sapiens
accccatccgctggctctcacccctcggagacgctcgcccgacagcatagtacttgccgcccagccacgcccgcgcgccagccaccatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcggcgctgcgacactacatcaacctcatcaccaggcagagatatggaaaacgatccagcccagagacactgatttcagacctcttgatgagagaaagcacagaaaatgttcccagaactcggcttgaagaccctgcaatgtggtgatgggaaatgagacttgctctctggccttttcctattttcagcccatatttcatcgtgtaaaacgagaatccacccatcctaccaatgcatgcagccactgtgctgaattctgcaatgttttcctttgtcatcattgtatatatgtgtgtttaaataaagtatcatgcatt

SEQ ID NO: 85 - DNA, 469 bp, Homo sapiens
accccatccgctggctctcacccctcggagacgctcgcccgacagcatagtacttgccgcccagccacgcccgcgcgccagccaccatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcggcgctgcgacactacatcaacctcatcaccaggcagaggcttgaagaccctgcaatgtggtgatgggaaatgagacttgctctctggccttttcctattttcagcccatatttcatcgtgtaaaacgagaatccacccatcctaccaatgcatgcagccactgtgctgaattctgcaatgttttcctttgtcatcattgtatatatgtgtgtttaaataaagtatcatgcatt

SEQ ID NO: 86 - protein, 97 aa, Homo sapiens
MLGNKRLGLSGLTLALSLLVCLGALAEAYPSKPDNPGEDAPAEDMARYYSALRHYINLITRQRYGKRSSPETLISDLLMRESTENVPRTRLEDPAMW

SEQ ID NO: 87 - protein, 70 aa, Homo sapiens
MLGNKRLGLSGLTLALSLLVCLGALAEAYPSKPDNPGEDAPAEDMARYYSALRHYINLITRQRLEDPAMW

SEQ ID NO: 88 - DNA, 411 bp, Homo sapiens
atggctgccgcacgcctctgcctctccctgctgctcctgtccacctgcgtggctctgttactacagccactgctgggtgcccagggagccccactggagccagtgtacccaggggacaatgccacaccagagcagatggcccagtatgcagctgatctccgtagatacatcaacatgctgaccaggcctaggtatgggaaaagacacaaagaggacacgctggccttctcggagtgggggtccccgcatgctgctgtccccagggagctcagcccgctggacttataatgccaccttctgtctcctacgactccatgagcagcgccagcccagctctcccctctgcacccttggctctggccaaagcttgctccctgctcccacacaggctcaataaagcaagtcaaagcc

SEQ ID NO: 89 - DNA, 339 bp, Homo sapiens
atggctgccgcacgcctctgcctctccctgctgctcctgtccacctgcgtggctctgttactacagccactgctgggtgcccagggagccccactggagccagtgtacccaggggacaatgccacaccagagcagatggcccagtatgcagctgatctccgtagatacatcaacatgctgaccaggcctagggagctcagcccgctggacttataatgccaccttctgtctcctacgactccatgagcagcgccagcccagctctcccctctgcacccttggctctggccaaagcttgctccctgctcccacacaggctcaataaagcaagtcaaagcc

SEQ ID NO: 90 - protein, 95 aa, Homo sapiens
MAAARLCLSLLLLSTCVALLLQPLLGAQGAPLEPVYPGDNATPEQMAQYAADLRRYINMLTRPRYGKRHKEDTLAFSEWGSPHAAVPRELSPLDL

SEQ ID NO: 91 - protein, 71 aa, Homo sapiens
MAAARLCLSLLLLSTCVALLLQPLLGAQGAPLEPVYPGDNATPEQMAQYAADLRRYINMLTRPRELSPLDL

SEQ ID NO: 92 - DNA, 1462 bp, Homo sapiens
gattactagttagcaaatgagggggtaaatattccagtggatacaagcttggactcttttcttgaagctttctttctatcagaagcatttgctgatattgctgacattgaaacattaaaagaaaatttgagaagcaatgggcatcctgaagctgcaagtatttctcattgtgctctctgttgcattgaaccatctgaaagctacacccattgaaagtcatcaggtggaaaagcggaaatgcaacactgccacatgtgcaacgcagcgcctggcaaattttttagttcattccagcaacaactttggtgccattctctcatctaccaacgtgggatccaatacatatggcaagaggaatgcagtagaggttttaaagagagagccactgaattacttgcccctttagaggacaatgtaactctatagttattgttttatgttctagtgatttcctgtataatttaacagtgcccttttcatctccagtgtgaatatatggtctgtgtgtctgatgtttgttgctaggacatataccttctcaaaagattgttttatatgtagtactaactaaggtcccataataaaaagatagtatcttttaaaatgaaatgtttttgctatagatttgtattttaaaacataagaacgtcattttgggacctatatctcagtggcacaggtttaagaacgaaggagaaaaaggtagtttgaaccttggtaaattgtaaacagctaataatgaagttattcttgacatgagaaaatcagtaattggaccaggcgcggtggctcttgcctgtagtcccggcactttgggaggccgaggcaggcagatcacaaggtcaggagttcgagaccagcctgaccaacgtggtgaaaccctgtctctactggaaatacggaaattagccgggggtggtgacatgtgcctgtaatcccagctactcaggaggctaaggcaggagaatcgcttgaacccaggaggcggaggttgcagtgagccgagattgcaccactgcactccagcctgggtggcagagtgagactcgtctcaaaaaaaagaaagaaaattagtaattgtaagtacccctgataagcaaattagtaattgtcaatacccctgttaagcaattcctttttgcagtatatttctgaaatgacagaatgctgttttaaaaacaaagaaataaaatcctgctcctgactcggtcaaaatattttttaaagtctattgtttgttgtgcttgctggtactaagaggctatttaaaagtataaaactgctttgtatccatgagggtttcattgtgtgttagcagcagtgagcttctattaaatgtatatgtcatttattttgtttaagtggctttcagcaaacctcagtcatattcttatgcagggtattgcgaaacaacttgtgttctattaatcgtgtcttcaattaaaagaccacagacttctgg

SEQ ID NO: 93 - protein, 89 aa, Homo sapiens
MGILKLQVFLIVLSVALNHLKATPIESHQVEKRKCNTATCATQRLANFLVHSSNNFGAILSSTNVGSNTYGKRNAVEVLKREPLNYLPL

SEQ ID NO: 94 - DNA, 2048 bp, Homo sapiens
gtcagagctgagaaaggtgtgaggggtatataagagctggattactagttagcaaatgagggggtaaatattccagtggatacaagcttggactcttttcttgaagctttctttctatcagaagcatttgctgatattgctgacattgaaacattaaaagaaaatttgagaagcaatgggcatcctgaagctgcaagtatttctcattgtgctctctgttgcattgaaccatctgaaagctacacccattgaaagatgtttggaccaaattccaatttttactgtttttcaagaaaatcatcaggtggaaaagcggaaatgcaacactgccacatgtgcaacgcagcgcctggcaaattttttagttcattccagcaacaactttggtgccattctctcatctaccaacgtgggatccaatacatatggcaagaggaatgcagtagaggttttaaagagagagccactgaattacttgcccctttagaggacaatgtaactctatagttattgttttatgttctagtgatttcctgtataatttaacagtgcccttttcatctccagtgtgaatatatggtctgtgtgtctgatgtttgttgctaggacatataccttctcaaaagattgttttatatgtagtactaactaaggtcccataataaaaagatagtatcttttaaaatgaaatgtttttgctatagatttgtattttaaaacataagaacgtcattttgggacctatatctcagtggcacaggtttaagaacgaaggagaaaaaggtagtttgaaccttggtaaattgtaaacagctaataatgaagttattcttgacatgagaaaatcagtaattggaccaggcgcggtggctcttgcctgtaatcccagcactttgggaggccgaggcaggcagatcacaaggtcaggagttcgagaccagcctgaccaacatggtgaaaccctgtctctactaaaaatacaaaaattagccgggggtggtgacatgtgcctgtaatcccagctactcaggaggctaaggcaggagaatcgcttaaacccaggaggcggaggttgcagtgagccgagattgcaccactgcactccagcctgggtggcagagtgagactcgtctcaaaaaaaagaaagaaaattagtaattgtaagtacccctgataagcaaattagtaattgtcaatacccctgttaagcaattcctttttgcagtatatttctgaaatgacagaatgctgttttaaaaacaaagaaataaaatcctgctcctgactcggtcaaaatattttttaaagtctattgtttgttgtgcttgctggtactaagaggctatttaaaagtataaaactgctttgtatccatgagggtttcattgtgtgttagcagcagtgagcttctattaaatgtatatgtcatttattttgtttaagtggctttcagcaaacctcagtcatattcttatgcagggtattgcgaaacaacttgtgttctattaatcgtgtcttcaattaaaagaccacagacttctggaaactctttgctgtataagaattatttcttttgtttaacaaattagacatttctggcagaggttatgtatatgatacactttttttgatagcagctgcaatgttggacagaagatgaaatgctttgctttgagtcagattcttatgaatatctgcttttccctgactttgagttaggtagctttggaagtagcattaattcagataaactgccatcatgctgcgttatgccatttctaaagacactcaacttgtacttttaaaaaaatagaaaaaataagcatttcaatctaagtggaaatttgactcattgacttacatttctaagttaaaatttccctttatgaagtgtgccttaggttaccaaattgtagaggctttcgttggtggtggtaagtggtagcggtagtgagtgtatagaggcagggaaatatatttataataaattctatgtcatgaattacatattgaaataaataggtgaatatacaaatttaaaaaagtga

SEQ ID NO: 95 - protein, 103 aa, Homo sapiens
MGILKLQVFLIVLSVALNHLKATPIERCLDQIPIFTVFQENHQVEKRKCNTATCATQRLANFLVHSSNNFGAILSSTNVGSNTYGKRNAVEVLKREPLNYLPL

SEQ ID NO: 96 - DNA, 2098 bp, Homo sapiens
gtcagagctgagaaaggtgtgaggggtatataagagctggattactagttagcaaatgagggggtaaatattccagtggatacaagcttggactcttttcttgaagctttctttctatcagaagcatttgctgatattgctgacattgaaacattaaaagaaaatttgagaagcaatgggcatcctgaagctgcaagtatttctcattgtgctctctgttgcattgaaccatctgaaagctacacccattgaaagacaggaatggataattccagttttgtcaagaaatatactcttggaacttagaggggcaaagccagaacatgaagcgggaaaaaaatcaaaagtcatcaggtggaaaagcggaaatgcaacactgccacatgtgcaacgcagcgcctggcaaattttttagttcattccagcaacaactttggtgccattctctcatctaccaacgtgggatccaatacatatggcaagaggaatgcagtagaggttttaaagagagagccactgaattacttgcccctttagaggacaatgtaactctatagttattgttttatgttctagtgatttcctgtataatttaacagtgcccttttcatctccagtgtgaatatatggtctgtgtgtctgatgtttgttgctaggacatataccttctcaaaagattgttttatatgtagtactaactaaggtcccataataaaaagatagtatcttttaaaatgaaatgtttttgctatagatttgtattttaaaacataagaacgtcattttgggacctatatctcagtggcacaggtttaagaacgaaggagaaaaaggtagtttgaaccttggtaaattgtaaacagctaataatgaagttattcttgacatgagaaaatcagtaattggaccaggcgcggtggctcttgcctgtaatcccagcactttgggaggccgaggcaggcagatcacaaggtcaggagttcgagaccagcctgaccaacatggtgaaaccctgtctctactaaaaatacaaaaattagccgggggtggtgacatgtgcctgtaatcccagctactcaggaggctaaggcaggagaatcgcttaaacccaggaggcggaggttgcagtgagccgagattgcaccactgcactccagcctgggtggcagagtgagactcgtctcaaaaaaaagaaagaaaattagtaattgtaagtacccctgataagcaaattagtaattgtcaatacccctgttaagcaattcctttttgcagtatatttctgaaatgacagaatgctgttttaaaaacaaagaaataaaatcctgctcctgactcggtcaaaatattttttaaagtctattgtttgttgtgcttgctggtactaagaggctatttaaaagtataaaactgctttgtatccatgagggtttcattgtgtgttagcagcagtgagcttctattaaatgtatatgtcatttattttgtttaagtggctttcagcaaacctcagtcatattcttatgcagggtattgcgaaacaacttgtgttctattaatcgtgtcttcaattaaaagaccacagacttctggaaactctttgctgtataagaattatttcttttgtttaacaaattagacatttctggcagaggttatgtatatgatacactttttttgatagcagctgcaatgttggacagaagatgaaatgctttgctttgagtcagattcttatgaatatctgcttttccctgactttgagttaggtagctttggaagtagcattaattcagataaactgccatcatgctgcgttatgccatttctaaagacactcaacttgtacttttaaaaaaatagaaaaaataagcatttcaatctaagtggaaatttgactcattgacttacatttctaagttaaaatttccctttatgaagtgtgccttaggttaccaaattgtagaggctttcgttggtggtggtaagtggtagcggtagtgagtgtatagaggcagggaaatatatttataataaattctatgtcatgaattacatattgaaataaataggtgaatatacaaatttaaaaaagtga

SEQ ID NO: 97 - protein, 79 aa, Homo sapiens
MGILKLQVFLIVLSVALNHLKATPIERQEWIIPVLSRNILLELRGAKPEHEAGKKSKVIRWKSGNATLPHVQRSAWQIF

SEQ ID NO: 98 - protein, 15 aa, Artificial
  synthetic peptide
RCLDQIPIFTVFQEN

SEQ ID NO: 99 - protein, 53 aa, Artificial
  synthetic peptide
RQEWIIPVLSRNILLELRGAKPEHEAGKKSKVIRWKSGNATLPHVQRSAWQIF

SEQ ID NO: 100 - protein, 36 aa, Homo sapiens
YPSKPDNPGEDAPAEDMARYYSALRHYINLITRQRY

SEQ ID NO: 101 - protein, 42 aa, Homo sapiens
YPSKPDNPGEDAPAEDMARYYSALRHYINLITRQRLEDPAMW

SEQ ID NO: 102 - protein, 34 aa, Homo sapiens
SKPDNPGEDAPAEDMARYYSALRHYINLITRQRY

SEQ ID NO: 103 - protein, 40 aa, Homo sapiens
SKPDNPGEDAPAEDMARYYSALRHYINLITRQRLEDPAMW

SEQ ID NO: 104 - protein, 36 aa, Homo sapiens
APLEPVYPGDNATPEQMAQYAADLRRYINMLTRPRY

SEQ ID NO: 105 - protein, 42 aa, Homo sapiens
APLEPVYPGDNATPEQMAQYAADLRRYINMLTRPRELSPLDL

SEQ ID NO: 106 - protein, 34 aa, Homo sapiens
LEPVYPGDNATPEQMAQYAADLRRYINMLTRPRY

SEQ ID NO: 107 - protein, 40 aa, Homo sapiens
LEPVYPGDNATPEQMAQYAADLRRYINMLTRPRELSPLDL

SEQ ID NO: 108 - DNA, 777 bp, Gallus gallus
aaaagggatactccagagctaggtgcaagcgtggaactgtggcatgcacccttctcactaacgcaggcgtgcttctccagttagctccctgaccattagaagaaagtttggaagcaaagatgtgcaacctaaagctgtcagctttcttcattgtactttctgtcacgctgaactgtttggaagctacatctattgagaaattattatctgtgactgacgatctctctgatgggacttccaagaggcaagaatggatactgcccataatgtcacagaatacactctcaggacttagtgaggaaatgccggaacagccagcagcaaagacaaaaagtagtcaccaactggagaaacggaagtgcaacactgctacatgtgtgacacaacgcttggctgacttcttagttcgttccagcagcaacatcggagcaatttattcacctactaatgtggggtccaatacatatggaaagagggacacagctgggctttcaagcagaaaatcccaaaacaatacaaagctttagggtgttttaatgactatcaatacagttttcattaggaactcagtttctaatgtatcaataaccttttggtcatttattacttgtacagtcttaacagtgtccttgttttctttgtgtacgtgtatgtaatttatgcatgtataacatcatgcataggctatcgtttcaaatcctttaagaactccagaagtctgcttgtcaagttcctttgtaaaaaacaaaaaacaataataaatggcagaataattcc

SEQ ID NO: 109 - protein, 9 aa, Artificial
  synthetic peptide
CRSEGPDLW

SEQ ID NO: 110 - protein, 16 aa, Artificial
  synthetic peptide
RQRSEGGCGGRQRSEG

SEQ ID NO: 111 - DNA, 711 bp, Homo sapiens
ggaagtattacattttataagaatgtatttatgcagaggaggcttcaggtcgacctgtggcagtccagcccttgggggttccctcgccttccacctcctgctcatctgcttcactagctatccctatggtgtcggtgtgcaggccgtggcctgctgtggccatagcacttctggctctgctggtctgcctgggggcgctggtcgacacctgccccatcaaacccgaggctcctggcgaagacgagtccctggaggagctgagccactattatgcttccctgtgccactacctcaacgtggtcaccagacagtggtgggagggtgcagacatgtggtgaggacccctgaggtctccttggggatctgctaaccgcacccacctgatttgcatatccacttcctacccgggaaacacggattccccacctggccatcagggtctgggcaggattcaggtggggacttccacccgcatctcggtaccccctccccctgggctggagggctgtgtatggtccttccctggtcccaaaataaagagcaaattccacaggaaaagaaaataatgcatttatgcattacatgtaggaacaatatttaaaataacgaacccattcttctcgcttcctccaaacaccccttctggccacagtgataagttcaggaatatgatctcagccctggcctaagtcattcagcaactaaaagtca

SEQ ID NO: 112 - protein, 7 aa, Artificial
  synthetic peptide
SEGPDLW

SEQ ID NO: 113 - protein, 7 aa, Artificial
  synthetic peptide
LEDPAMW

SEQ ID NO: 114 - protein, 7 aa, Artificial
  synthetic peptide
ELSPLDL

SEQ ID NO: 115 - DNA, 1069 bp, Homo sapiens
gcccctggaggaactgaacccactatcggtcatggggccgagactaaatgtggcgggttgtctttaatctgctgccaagaggaaactcattcaggcaagttcagccctttatgaggaattcccctgtggtcacattccaattcctggacctgctgccaccctcagaactgcatgctccttcttcagactttctaagaatgactcaggtcattggtggagtgaagtcaagatttccaactcagtcacctgaagagatggagataccattcatggagctggaggtccctggagatttgggaattcagataacaagctaagataaggagtttgcctacctctgtcctagagcgaagcctgagccttgggcgcgcagcacaccacaagtatctgttactgtgttttgcagaagcttcaggcggggatataaaccccacaaggaaagcgctgagcagaggaggcctcagcttgacctgcggcagtgcagcccttgggacttccctcgccttccacctcctgctcgtctgcttcacaagctatcgctatggtgttcgtgcgcaggccgtggcccgccttgaccacagtgcttctggccctgctcgtctgcctaggggcgctggtcgacgcctaccccatcaaacccgaggctcccggcgaagacgcctcgccggaggagctgaaccgctactacgcctccctgcgccactacctcaacctggtcacccggcagcggtatgggaaaagagacggcccggacaggcttctttccaaaacgttcttccccgacggcgaggaccgccccgtcaggtcgcggtcggagggcccagacctgtggtgaggacccctgaggcctcctgggagatctgccaaccacgcccacgtcatttgcatacgcactcccgaccccagaaacccggattctgcctcccgacggcggcgtctgggcagggttcgggtgcggccctccgcccgcgtctcggtgcccccgccccctgggctggagggctgtgtgtggtccttccctggtcccaaaataaagagcaaattccacagaaaaaaaaaaaaaaaaaaa

SEQ ID NO: 116 - DNA, 551 bp, Homo sapiens
accccatccgctggctctcacccctcggagacgctcgcccgacagcatagtacttgccgcccagccacgcccgcgcgccagccaccatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcggcgctgcgacactacatcaacctcatcaccaggcagagatatggaaaacgatccagcccagagacactgatttcagacctcttgatgagagaaagcacagaaaatgttcccagaactcggcttgaagaccctgcaatgtggtgatgggaaatgagacttgctctctggccttttcctattttcagcccatatttcatcgtgtaaaacgagaatccacccatcctaccaatgcatgcagccactgtgctgaattctgcaatgttttcctttgtcatcattgtatatatgtgtgtttaaataaagtatcatgcattc

SEQ ID NO: 117 - DNA, 527 bp, Homo sapiens
atggctgccgcacgcctctgcctctccctgctgctcctgtccacctgcgtggctctgttactacagccactgctgggtgcccagggagccccactggagccagtgtacccaggggacaatgccacaccagagcagatggcccagtatgcagctgatctccgtagatacatcaacatgctgaccaggcctaggtatgggaaaagacacaaagaggacacgctggccttctcggagtgggggtccccgcatgctgctgtccccagggagctcagcccgctggacttataatgccaccttctgtctcctacgactccatgagcagcgccagcccagctctcccctctgcacccttggctctggccaaagcttgctccctgctcccacacaggctcaataaagcaagtcaaagccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 118 - DNA, 22 bp, Artificial
  synthetic oligonucleotide primer
ctatcgctatggtgttcgtgcg

SEQ ID NO: 119 - DNA, 18 bp, Artificial
  synthetic oligonucleotide primer
caccacaggtctgggccc

SEQ ID NO: 120 - DNA, 15 bp, Artificial
  synthetic oligonucleotide primer
acccgggcgaggacg

SEQ ID NO: 121 - DNA, 24 bp, Artificial
  synthetic oligonucleotide primer
cgttttacacgatgaaatatgggc

SEQ ID NO: 122 - DNA, 18 bp, Artificial
  synthetic oligonucleotide primer
gcacgcctctgcctctcc

SEQ ID NO: 123 - DNA, 17 bp, Artificial
  synthetic oligonucleotide primer
gctggcgctgctcatgg

SEQ ID NO: 124 - DNA, 719 bp, Homo sapiens
ctcagcttgacctgcggcagtgcagcccttgggacttccctcgccttccacctcctgctcgtctgcttcacaagctatcgctatggtgttcgtgcgcaggccgtggcccgccttgaccacagtgcttctggccctgctcgtctgcctaggggcgctggtcgacgcctaccccatcaaacccgaggctccccgcgaagacgcctcgccggaggagctgaaccgctactacgcctccctgcgccactacctcaacctggtcacccggcagcggtatgggaaaagagacggcccggacacgcttctttccaaaacgttcttccccgacggcgaggaccgccccgtcaggtcgcggtaaaagcgcccgttaccacacatcctgcatccgagagcgcggcctggccctaccctggcaacatcacttaacgacgtctcccaggctcgcctccccagatccaattccttccccttcgcttccgcaggtcggagggcccagacctgtggtgaggacccctgaggcctcctgggagatctgccaaccacgcccacgtcatttgcatacgcactcccgaccccagaaacccggattctgcctcccgacggcggcgtctgggcagggttcgggtgcggccctccgcccgcgtctcggtgcccccgccccctgggctggagggctgtgtgtggtccttccctggtcccaaaataaagagcaaattccacag